(19) United States
(12) Patent Application Publication
(10) Pub. No.: US 2022/0007777 A1
(43) Pub. Date: Jan. 13, 2022

(54) METHODS OF GENERATING MYCELIUM MATERIALS WITH IMPROVED PROPERTIES

(71) Applicant: Bolt Threads, Inc., Emeryville, CA (US)

(72) Inventors: Jessica WANG, Oakland, CA (US); Ritu Bansal MUTALIK, Albany, CA (US); Matthew Jordan SMITH, Emeryville, CA (US); Nicole Elizabeth SUBLER, Berkeley, CA (US); La'Deva MCKENZIE, Northampton (GB); Isaac Samuel COLLINS, Hanslope (GB); Karl FLOWERS, Northampton (GB); Victoria ADDY, Northampton (GB); Jamie McAusland BAINBRIDGE, Berkeley, CA (US); Mitchell Joseph HEINRICH, Oakland, CA (US)

(21) Appl. No.: 17/293,384

(22) PCT Filed: Nov. 14, 2019
(86) PCT No.: PCT/US2019/061500

§ 371 Date: May 12, 2021

Related U.S. Application Data
(60) Provisional application No. 62/782,277, filed on Dec. 19, 2018.
(60) Provisional application No. 62/767,433, filed on Nov. 14, 2018.

(51) Int. Cl. A43B 1/02; C07K 14/415; A43B 23/02; D06N 3/00

(52) U.S. Cl. CPC A43B 1/02; C07K 14/415; D06N 2211/106; D06N 3/0015; A43B 23/0205

(57) ABSTRACT

Provided herein are compositions of mycelium material, and methods for production thereof. Also provided herein are articles of footwear includes an upper, a lasting board affixed with the upper to define an interior foot-receiving cavity therewith, and an outsole coupled with the upper opposite the lasting board. The upper includes at least a portion of a mycelium material that includes one or more proteins derived from an organism other than mycelium.

### CROSS-REFERENCE TO RELATED APPLICATIONS

This application claims the benefit of U.S. Provisional Application No. 62/767,433, filed Nov. 14, 2018, and U.S. Provisional Application 62/782,277, filed Dec. 19, 2018, the contents of which are incorporated by reference in their entirety.

### BACKGROUND

Due to its bioefficiency, strength and low environmental footprint, mycelium is of increasing interest in the next generation of sustainable materials. To this end, various applications have discussed various methods of growing networks of enmeshed mycelium both on its own and as a composite material (i.e. enmeshed with particles, fibers or networks of fibers). However, the mycelium materials currently undergoing development have poor mechanical qualities, including increased delamination and tearing under stress, and poor aesthetic qualities. What is needed, therefore, are improved mycelium materials with favorable mechanical properties, aesthetic properties, and other advantages, as well as materials and methods for making improved mycelium materials.

### SUMMARY

In one aspect, provided herein are compositions comprising a cultivated mycelium material and one or more proteins, wherein the one or more proteins are from a species other than a fungal species from which the cultivated mycelium material is generated.

In some embodiments, the one or more proteins are from a plant source.

In some embodiments, the plant source is a pea plant.

In some embodiments, the plant source is a soybean plant.

In some embodiments, the composition comprises a dye.

In some embodiments, the dye is selected from the group consisting of: an acid dye, a direct dye, a synthetic dye, a natural dye, and a reactive dye.

In some embodiments, the composition comprises a plasticizer.

In some embodiments, the plasticizer is selected from the group consisting of oil, glycerin and fat liquor.

In some embodiments, the composition is flexible.

In some embodiments, the one or more proteins are crosslinked.

In some embodiments, the one or more proteins are crosslinked with transglutaminase.

In some embodiments, the composition comprises an enzyme.

In some embodiments, the enzyme comprises transglutaminase.

In another aspect, provide herein are compositions comprising a cultivated mycelium material colored with a dye to produce a color, and wherein the color of the cultivated mycelium material is substantially uniform on one or more surfaces of the cultivated mycelium material.

In some embodiments, the dye is selected from the group consisting of: an acid dye, a direct dye, a synthetic dye, a natural dye, and a reactive dye.

In some embodiments, the composition comprises one or more proteins that are from a species other than a fungal species from which the cultivated mycelium material is generated.

In some embodiments, the one or more proteins are from a plant source.

In some embodiments, the plant source is a pea plant.

In some embodiments, the plant source is a soybean plant.

In some embodiments, the dye is penetrated throughout the interior of the composition.

In some embodiments, the composition comprises a plasticizer.

In some embodiments, the plasticizer is selected from the group consisting of oil, glycerin, and fat liquor.

In some embodiments, the composition is flexible.

In some embodiments, the composition comprises tannins.

In some embodiments, the composition comprises a finishing agent applied to one or more surfaces of the composition.

In some embodiments, the finishing agent is selected from the group consisting of: urethane, wax, nitrocellulose, or a plasticizer.

In another aspect, provide herein are methods, comprising: generating a cultivated mycelium material; contacting the cultivated mycelium material with a solution comprising one or more proteins to produce a composition comprising the cultivated mycelium material and one or more proteins, wherein the one or more proteins are from a species other than a fungal species from which the cultivated mycelium material is generated; and pressing the cultivated mycelium material.

In some embodiments, the contacting comprises submerging the cultivated mycelium material in the solution.

In some embodiments, the contacting comprises contacting the cultivated mycelium material with the solution in a single step.

In some embodiments, the contacting comprises contacting the cultivated mycelium material with the solution in one or more steps.

In some embodiments, the one or more proteins are from a plant source.

In some embodiments, the plant source is a pea plant.

In some embodiments, the plant source is a soybean plant.

In some embodiments, the solution comprises a dye.

In some embodiments, the composition is colored with the dye to produce a color, and the color of the cultivated mycelium material is substantially uniform on one or more surfaces of the cultivated mycelium material.

In some embodiments, the dye is penetrated throughout the interior of the composition.

In some embodiments, the dye is selected from the group consisting of: an acid dye, a direct dye, a synthetic dye, a natural dye, and a reactive dye.

In some embodiments, the solution comprises a plasticizer.

In some embodiments, the plasticizer is selected from the group consisting of oil, glycerin, and fat liquor.

In some embodiments, the composition is flexible.

In some embodiments, the one or more proteins are crosslinked.

In some embodiments, one or more proteins are crosslinked with transglutaminase.

In some embodiments, the solution comprises an enzyme.

In some embodiments, the enzyme comprises transglutaminase.

In some embodiments, the pressing comprises pressing the cultivated mycelium material to a thickness of 0.1 inch to 0.5 inch.

In some embodiments, the pressing comprises pressing the cultivated mycelium material to a thickness of 0.25 inch.

In some embodiments, the pressing is repeated one or more times.

In some embodiments, the pressing comprises pressing the cultivated mycelium material to a thickness of 0.25 inch.

In some embodiments, the pressing comprises pressing the cultivated mycelium material with a roller.

In some embodiments, the solution comprises tannins.

In some embodiments, the method further comprises incubating the composition.

In some embodiments, the incubating comprises incubating the composition at a set temperature for a set amount of time.

In some embodiments, the set temperature is 40° C.

In some embodiments, the method further comprising drying the composition.

In some embodiments, the method further comprises applying a finishing agent to one or more surfaces of the composition.

In some embodiments, the finishing agent is selected from the group consisting of: urethane, wax, nitrocellulose, or a plasticizer.

In another aspect, provided herein are articles of footwear, comprising: an upper; a lasting board affixed with the upper to define an interior foot-receiving cavity therewith; an outsole coupled with the upper opposite the lasting board; wherein the upper includes at least a portion of a mycelium material that includes one or more proteins derived from an organism other than mycelium.

In some embodiments, the upper comprises a plurality of portions of the mycelium material in respective implementations thereof having different physical properties.

In some embodiments, the different physical properties are selected to correlate with desired characteristics of the corresponding locations of the portions within the upper.

In some embodiments, one of the portions of the mycelium material includes a vamp, the respective implementation of the mycelium material having higher relative flexibility compared to at least one of the portion.

In some embodiments, one of the portions of the mycelium material includes a heel counter, the respective implementation of the mycelium material having higher relative rigidity compared to at least one of the portion.

In some embodiments, the mycelium material is at least one of tanned and dyed to resemble leather.

In some embodiments, the article further includes a midsole affixed with the lasting board, the outsole being affixed with the midsole so as to be coupled with the upper.

In some embodiments, the upper comprises a plurality of discrete portions of the mycelium material.

In some embodiments, the portions are assembled together using at least one of: topstitching, felled stitching, and stitch and turn construction.

In some embodiments, the portions are assembled together using at least one of: solvent-based adhesive, UV curing adhesive, heat-activated adhesive, and water-based adhesive.

In some embodiments, at least one of the portions is split to resemble suede leather.

In some embodiments, at least one of the portions includes an edge thinned by skivving.

In some embodiments, the portions are assembled together using heat bonding.

In some embodiments, the upper further includes at least one additional portion of a textile material.

In some embodiments, the textile material is thermoplastic and is affixed with at least one of the portions of the mycelium material by heat bonding.

In some embodiments, the upper includes interfacing assembled with a portion thereof.

In some embodiments, perforations along a portion thereof.

In some embodiments, the perforations vary in at least one of size and relative spacing over an area of the upper.

In some embodiments, the upper is laser etched along a portion thereof.

In some embodiments, the upper includes at least one reinforcing portion injection molded thereon.

In some embodiments, the upper includes at least one 3-D printed element fused therewith.

In some embodiments, the at least a portion of the upper includes at least one portion molded in a three dimensional shape.

In some embodiments, the upper is comprised of a single molded piece of the mycelium material.

In some embodiments, the mycelium material includes a plurality of bonded layers of the mycelium material in respective implementations thereof having different physical properties.

In some embodiments, at least one of the lasting board and the outsole includes at least a portion of the mycelium material.

In another aspect, provide herein are athletic sneakers, comprising: an upper including at least a portion of a mycelium material that includes one or more proteins derived from an organism other than mycelium; a lasting board affixed with the upper to define an interior foot-receiving cavity therewith; a midsole of a foam material and affixed with the lasting board; and an outsole of a rubber material and affixed with the midsole opposite the lasting board; wherein the mycelium material is at least one of tanned and dyed to resemble leather, and the upper is configured and assembled to resemble athletic footwear of leather.

In another aspect, provide herein are athletic sneakers, comprising: an upper including at least a portion of a mycelium material that includes one or more proteins derived from an organism other than mycelium; a lasting board affixed with the upper to define an interior foot-receiving cavity therewith; a midsole of a foam material and affixed with the lasting board; and an outsole of a rubber material and affixed with the midsole opposite the lasting board; wherein the upper includes at least one portion molded in a three dimensional shape.

### BRIEF DESCRIPTION OF THE DRAWINGS

The foregoing summary, as well as the following detailed description, will be better understood when read in conjunction with the appended drawings. For the purpose of illustration, there are shown in the drawings, certain aspects of the disclosure. It should be understood, however, that the disclosure is not limited to the precise arrangements and instrumentalities shown. Drawings are not necessarily to scale. Certain features may be exaggerated in scale or shown in schematic form in the interest of clarity and conciseness.

FIG. 1 is a front perspective view of an athletic sneaker according to an aspect of the disclosure.

FIG. 2 is a front perspective exploded view of the athletic sneaker.

FIG. 3 is a front perspective exploded view of an upper of the athletic sneaker.

FIG. 4 is front perspective view of an athletic sneaker according to another aspect of the disclosure.

FIG. 5 is a front perspective exploded view of the athletic sneaker.

FIG. 6 is a top plan view of a cut sheet of mycelium material useable to fabricate an upper of the athletic sneaker.

FIG. 7 is a front perspective view of an article of footwear according to another aspect of the disclosure.

FIG. 8 shows a cross section of an exemplary cultivated mycelium material after the indicated dye and treatment process.

FIG. 9 shows a cross section of an exemplary cultivated mycelium material after the indicated dye and treatment process.

FIG. 10 shows a cross section of an exemplary cultivated mycelium material after the indicated dye and treatment process.

FIG. 11 shows a cross section of an exemplary cultivated mycelium material after the indicated dye and treatment process.

FIG. 12 shows a cross section of an exemplary cultivated mycelium material after the indicated dye and treatment process.

FIG. 13 shows a cross section of an exemplary cultivated mycelium material after the indicated dye and treatment process.

FIG. 14 shows a cross section of an exemplary cultivated mycelium material after the indicated dye and treatment process.

FIG. 15 shows a cross section of an exemplary cultivated mycelium material after the indicated dye and treatment process.

FIG. 16 shows a cross section of an exemplary cultivated mycelium material after the indicated dye and treatment process.

FIG. 17 shows a cross section of an exemplary cultivated mycelium material after the indicated dye and treatment process.

FIG. 18 shows a cross section of an exemplary cultivated mycelium material after the indicated dye and treatment process.

FIG. 19 shows a cross section of an exemplary cultivated mycelium material after the indicated dye and treatment process.

FIG. 20 shows a cross section of an exemplary cultivated mycelium material after the indicated dye and treatment process.

FIG. 21 shows a cross section of an exemplary cultivated mycelium material after the indicated dye and treatment process.

FIG. 22 shows a cross section of an exemplary cultivated mycelium material after the indicated dye and treatment process.

FIG. 23 shows a cross section of an exemplary cultivated mycelium material after the indicated dye and treatment process.

FIG. 24 shows a cross section of an exemplary cultivated mycelium material after the indicated dye and treatment process.

FIG. 25 shows a cross section of an exemplary cultivated mycelium material after the indicated dye and treatment process.

FIG. 26 shows a cross section of an exemplary cultivated mycelium material after the indicated dye and treatment process.

FIG. 27A shows a cross section of an exemplary cultivated mycelium material after the indicated dye and treatment process. FIG. 27B shows an exemplary cultivated mycelium material after a color fastness test and the indicated dye and treatment process.

FIG. 28A shows a cross section of an exemplary cultivated mycelium material after the indicated dye and treatment process. FIG. 28B shows an exemplary cultivated mycelium material after a color fastness test and the indicated dye and treatment process.

FIG. 29A shows a cross section of an exemplary cultivated mycelium material after the indicated dye and treatment process. FIG. 29B shows an exemplary cultivated mycelium material after a color fastness test and the indicated dye and treatment process.

FIG. 30A shows a cross section of an exemplary cultivated mycelium material after the indicated dye and treatment process. FIG. 30B shows an exemplary cultivated mycelium material after a color fastness test and the indicated dye and treatment process.

FIG. 31A shows a cross section of an exemplary cultivated mycelium material after the indicated dye and treatment process. FIG. 31B shows an exemplary cultivated mycelium material after a color fastness test and the indicated dye and treatment process.

FIG. 32A shows a cross section of an exemplary cultivated mycelium material after the indicated dye and treatment process. FIG. 32B shows an exemplary cultivated mycelium material after a color fastness test and the indicated dye and treatment process.

FIG. 33A shows a cross section of an exemplary cultivated mycelium material after the indicated dye and treatment process. FIG. 33B shows an exemplary cultivated mycelium material after a color fastness test and the indicated dye and treatment process.

FIG. 34A shows a cross section of an exemplary cultivated mycelium material after the indicated dye and treatment process. FIG. 34B shows an exemplary cultivated mycelium material after a color fastness test and the indicated dye and treatment process.

FIG. 35A shows a cross section of an exemplary cultivated mycelium material after the indicated dye and treatment process. FIG. 35B shows an exemplary cultivated mycelium material after a color fastness test and the indicated dye and treatment process.

FIG. 36A shows a cross section of an exemplary cultivated mycelium material after the indicated dye and treatment process. FIG. 36B shows an exemplary cultivated mycelium material after a color fastness test and the indicated dye and treatment process.

FIG. 37A shows a cross section of an exemplary cultivated mycelium material after the indicated dye and treatment process. FIG. 37B shows an exemplary cultivated mycelium material after a color fastness test and the indicated dye and treatment process.

FIG. 38A shows a cross section of an exemplary cultivated mycelium material after the indicated dye and treatment process. FIG. 38B shows an exemplary cultivated mycelium material after a color fastness test and the indicated dye and treatment process.

FIG. 39A shows a cross section of an exemplary cultivated mycelium material after the indicated dye and treatment process. FIG. 39B shows an exemplary cultivated mycelium material after a color fastness test and the indicated dye and treatment process.

FIG. 40A shows a cross section of an exemplary cultivated mycelium material after the indicated dye and treatment process. FIG. 40B shows an exemplary cultivated mycelium material after a color fastness test and the indicated dye and treatment process.

FIG. 41A shows a cross section of an exemplary cultivated mycelium material after the indicated dye and treatment process. FIG. 41B shows an exemplary cultivated mycelium material after a color fastness test and the indicated dye and treatment process.

FIG. 42A shows a cross section of an exemplary cultivated mycelium material after the indicated dye and treatment process. FIG. 42B shows an exemplary cultivated mycelium material after a color fastness test and the indicated dye and treatment process.

FIG. 43A shows a cross section of an exemplary cultivated mycelium material after the indicated dye and treatment process. FIG. 43B shows an exemplary cultivated mycelium material after a color fastness test and the indicated dye and treatment process.

FIG. 44A shows a cross section of an exemplary cultivated mycelium material after the indicated dye and treatment process. FIG. 44B shows an exemplary cultivated mycelium material after a color fastness test and the indicated dye and treatment process.

FIG. 45A shows a cross section of an exemplary cultivated mycelium material after the indicated dye and treatment process. FIG. 45B shows an exemplary cultivated mycelium material after a color fastness test and the indicated dye and treatment process.

FIG. 46A shows a cross section of an exemplary cultivated mycelium material after the indicated dye and treatment process. FIG. 46B shows an exemplary cultivated mycelium material after a color fastness test and the indicated dye and treatment process.

FIG. 47A shows a cross section of an exemplary cultivated mycelium material after the indicated dye and treatment process. FIG. 47B shows an exemplary cultivated mycelium material after a color fastness test and the indicated dye and treatment process.

FIG. 48A shows a cross section of an exemplary cultivated mycelium material after the indicated dye and treatment process. FIG. 48B shows an exemplary cultivated mycelium material after a color fastness test and the indicated dye and treatment process.

FIG. 49A shows a cross section of an exemplary cultivated mycelium material after the indicated dye and treatment process. FIG. 49B shows an exemplary cultivated mycelium material after a color fastness test and the indicated dye and treatment process.

FIG. 50A shows a cross section of an exemplary cultivated mycelium material after the indicated dye and treatment process. FIG. 50B shows an exemplary cultivated mycelium material after a color fastness test and the indicated dye and treatment process.

FIG. 51A shows a cross section of an exemplary cultivated mycelium material after the indicated dye and treatment process. FIG. 51B shows an exemplary cultivated mycelium material after a color fastness test and the indicated dye and treatment process.

FIG. 52A shows a cross section of an exemplary cultivated mycelium material after the indicated dye and treatment process. FIG. 52B shows an exemplary cultivated mycelium material after a color fastness test and the indicated dye and treatment process.

FIG. 53A shows a cross section of an exemplary cultivated mycelium material after the indicated dye and treatment process. FIG. 53B shows an exemplary cultivated mycelium material after a color fastness test and the indicated dye and treatment process.

FIG. 54A shows a cross section of an exemplary cultivated mycelium material after the indicated dye and treatment process. FIG. 54B shows an exemplary cultivated mycelium material after a color fastness test and the indicated dye and treatment process.

FIG. 55A shows a cross section of an exemplary cultivated mycelium material after the indicated dye and treatment process. FIG. 55B shows an exemplary cultivated mycelium material after a color fastness test and the indicated dye and treatment process.

FIG. 56A shows a cross section of an exemplary cultivated mycelium material after the indicated dye and treatment process. FIG. 56B shows an exemplary cultivated mycelium material after a color fastness test and the indicated dye and treatment process.

FIG. 57A shows a cross section of an exemplary cultivated mycelium material after the indicated dye and treatment process. FIG. 57B shows an exemplary cultivated mycelium material after a color fastness test and the indicated dye and treatment process.

FIG. 58A shows a cross section of an exemplary cultivated mycelium material after the indicated dye and treatment process. FIG. 58B shows an exemplary cultivated mycelium material after a color fastness test and the indicated dye and treatment process.

FIG. 59A shows a cross section of an exemplary cultivated mycelium material after the indicated dye and treatment process. FIG. 59B shows an exemplary cultivated mycelium material after a color fastness test and the indicated dye and treatment process.

FIG. 60A shows a cross section of an exemplary cultivated mycelium material after the indicated dye and treatment process. FIG. 60B shows an exemplary cultivated mycelium material after a color fastness test and the indicated dye and treatment process.

FIG. 61A shows a cross section of an exemplary cultivated mycelium material after the indicated dye and treatment process. FIG. 61B shows an exemplary cultivated mycelium material after the indicated dye and treatment process.

FIG. 62A shows a cross section of an exemplary cultivated mycelium material after the indicated dye and treatment process. FIG. 62B shows an exemplary cultivated mycelium material after the indicated dye and treatment process.

FIG. 63A shows a cross section of an exemplary cultivated mycelium material after the indicated dye and treatment process. FIG. 63B shows an exemplary cultivated mycelium material after a color fastness test and the indicated dye and treatment process.

FIG. 64A shows a cross section of an exemplary cultivated mycelium material after the indicated dye and treatment process. FIG. 64B shows an exemplary cultivated mycelium material after a color fastness test and the indicated dye and treatment process.

FIG. 65A shows a cross section of an exemplary cultivated mycelium material after the indicated dye and treatment process. FIG. 65B shows an exemplary cultivated mycelium material after a color fastness test and the indicated dye and treatment process.

FIG. 66A shows a cross section of an exemplary cultivated mycelium material after the indicated dye and treatment process. FIG. 66B shows an exemplary cultivated mycelium material after a color fastness test and the indicated dye and treatment process.

FIG. 67A shows a cross section of an exemplary cultivated mycelium material after the indicated dye and treatment process. FIG. 67B shows an exemplary cultivated mycelium material after a color fastness test and the indicated dye and treatment process.

FIG. 68A shows a cross section of an exemplary cultivated mycelium material after the indicated dye and treatment process. FIG. 68B shows an exemplary cultivated mycelium material after the indicated dye and treatment process.

FIG. 69A shows a cross section of an exemplary cultivated mycelium material after the indicated dye and treatment process. FIG. 69B shows an exemplary cultivated mycelium material after the indicated dye and treatment process.

FIG. 70A shows a cross section of an exemplary cultivated mycelium material after the indicated dye and treatment process. FIG. 70B shows an exemplary cultivated mycelium material after the indicated dye and treatment process.

FIG. 71A shows a cross section of an exemplary cultivated mycelium material after the indicated dye and treatment process. FIG. 71B shows an exemplary cultivated mycelium material after the indicated dye and treatment process.

FIG. 72A shows a cross section of an exemplary cultivated mycelium material after the indicated dye and treatment process. FIG. 72B shows an exemplary cultivated mycelium material after the indicated dye and treatment process.

FIG. 73A shows a cross section of an exemplary cultivated mycelium material after the indicated dye and treatment process. FIG. 73B shows an exemplary cultivated mycelium material after the indicated dye and treatment process.

FIG. 74A shows a cross section of an exemplary cultivated mycelium material after the indicated dye and treatment process. FIG. 74B shows an exemplary cultivated mycelium material after the indicated dye and treatment process.

FIG. 75A shows a cross section of an exemplary cultivated mycelium material after the indicated dye and treatment process. FIG. 75B shows an exemplary cultivated mycelium material after the indicated dye and treatment process.

FIG. 76A shows a cross section of an exemplary cultivated mycelium material after the indicated dye and treatment process. FIG. 76B shows an exemplary cultivated mycelium material after the indicated dye and treatment process.

FIG. 77 shows an exemplary cultivated mycelium material after a nitrocellulose and protein polishable finish—box effect treatment.

FIG. 78 shows an exemplary cultivated mycelium material after a nitrocellulose finish—box effect treatment

FIG. 79 shows an exemplary cultivated mycelium material after conventional polyurethane finish treatment.

FIG. 80 shows an exemplary cultivated mycelium material after antique effect finish treatment.

FIG. 81 shows an exemplary cultivated mycelium material after distressed effect finish treatment.

FIG. 82 shows an exemplary cultivated mycelium material after embossed Luganil Olive Brown finish treatment.

FIG. 83A shows a cross section of an exemplary cultivated mycelium material after the indicated dye and treatment process. FIG. 83B shows an exemplary cultivated mycelium material after the indicated dye and treatment process.

FIG. 84A shows a cross section of an exemplary cultivated mycelium material after the indicated dye and treatment process. FIG. 84B shows an exemplary cultivated mycelium material after the indicated dye and treatment process.

FIG. 85A shows a cross section of an exemplary cultivated mycelium material after the indicated dye and treatment process. FIG. 85B shows an exemplary cultivated mycelium material after the indicated dye and treatment process.

FIG. 86A shows a cross section of an exemplary cultivated mycelium material after the indicated dye and treatment process. FIG. 86B shows an exemplary cultivated mycelium material after the indicated dye and treatment process.

FIG. 87 shows an exemplary mycelium material after a pea protein finish.

FIG. 88 shows an exemplary mycelium material after an unstirred soya protein finish.

FIG. 89 shows an exemplary mycelium material after a stirred soya protein finish.

FIG. 90 shows an exemplary mycelium material after a hemp protein finish.

FIG. 91 shows an exemplary mycelium material after a 50:50 pea protein to FI 50 finish.

FIG. 92 shows an exemplary mycelium material after a 50:50 soya protein to FI 50 finish.

FIG. 93 shows an exemplary mycelium material after a pea protein and crosslinker finish.

FIG. 94 shows an exemplary mycelium material after Luganil Brown dye and a carnauba flake wax finish.

FIG. 95 shows an exemplary mycelium material after Luganil Bordeaux dye, wash, and a carnauba flake wax finish.

FIG. 96 shows an exemplary mycelium material after Luganil Yellow dye, wash, and a carnauba liquid wax finish.

FIG. 97 shows an exemplary mycelium material after Luganil Brown dye, wash, and a carnauba liquid wax finish.

FIG. 98 shows an exemplary mycelium material after a waxy filler, water based PU, and carnauba flake wax finish.

FIG. 99 shows an exemplary mycelium material after a 1× coat of pea protein and crosslinker finish.

FIG. 100 shows an exemplary mycelium material after a 2× coat of pea protein and crosslinker finish.

FIG. 101 shows an exemplary mycelium material after a pea protein, crosslinker, and filler finish without embossing.

FIG. 102 shows an exemplary mycelium material after a pea protein, crosslinker, and filler finish with embossing.

FIG. 103 shows an exemplary mycelium material after Luganil Red dye, wash, and a pea protein and crosslinker finish.

FIG. 104 shows an exemplary mycelium material after Luganil Brown dye, and a glycerin soak, pea protein and crosslinker finish.

FIG. 105 shows an exemplary mycelium material after Luganil Bordeaux dye, and a pea protein and crosslinker finish.

### DETAILED DESCRIPTION

The details of various embodiments are set forth in the description below. It is also to be understood that the specific articles, components, and processes illustrated in the attached drawings, and described in the following specification are simply exemplary of the concepts defined in the appended claims. Hence, specific dimensions and other physical characteristics relating to the embodiments disclosed herein are not to be considered as limiting, unless the claims expressly state otherwise. Other features, objects, and advantages will be apparent from the description. Unless otherwise defined herein, scientific and technical terms shall have the meanings that are commonly understood by those of ordinary skill in the art. Further, unless otherwise required by context, singular terms shall include the plural and plural terms shall include the singular. The terms “a” and “an” includes plural references unless the context dictates otherwise. Generally, nomenclatures used in connection with, and techniques of, biochemistry, enzymology, molecular and cellular biology, microbiology, genetics, protein, and nucleic acid chemistry, and hybridization described herein, are those well-known and commonly used in the art.

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

An “isolated” organic molecule (e.g., a silk protein) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

An endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The twenty conventional amino acids and their abbreviations follow conventional usage. See Immunology—A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides described herein. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is sometimes also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A useful algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than BLASTp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

The terms “cultivate” and “cultivated” refer to the use of defined techniques to deliberately grow a fungus or other organism.

The term “hyphae” refers to a morphological structure of a fungus that is characterized by a branching filamentous shape.

The term “mycelium” refers to a structure formed by one or more masses of branching hyphae. Mycelium is a distinct and separate structure from a fruiting body of a fungus or sporocarp.

The term “cultivated mycelium material” refers to material that includes, in part, one or more masses of cultivated mycelium, or includes solely of cultivated mycelium. As used herein, the term “cultivated mycelium material” encompasses composite mycelium materials as defined below.

The term “composite mycelium material” refers to any mass of cultivated mycelium material that has been grown to enmesh with a second material. In some embodiments, the second material is embedded and/or entangled within a composite mycelium material. In some embodiments, the second material is positioned on one or more surfaces of the composite mycelium material. Suitable second materials, include but are not limited to, a textile, a mass of contiguous, disordered fibers (e.g. non-woven fibers), a perforated material (e.g. metal mesh, perforated plastic), a mass of discontiguous particles (e.g. pieces of woodchip) or any combination thereof. In specific embodiments, the second material is selected from the group consisting of a mesh, a cheesecloth, a fabric, a knit fiber, a woven fiber, and a non-woven fiber.

The term “plasticizer” as used herein refers to any molecule that interacts with a structure to increase mobility of the structure.

The term “processed mycelium material” as used herein refers to a mycelium that has been post-processed by any combination of treatments with preserving agents, plasticizers, finishing agents, dyes, and/or protein treatments.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range, is encompassed herein. The upper and lower limits of these smaller ranges may independently be included in the smaller ranges, and are also encompassed herein, subject to any specifically excluded limit in the stated range. Where the stated range includes one or both of the limits, ranges excluding either or both of those included limits are also included herein.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Exemplary methods and materials are described below, although methods and materials similar or equivalent to those described herein can also be used and will be apparent to those of skill in the art. All publications and other references mentioned herein are incorporated by reference in their entirety. In case of conflict, the present specification, including definitions, will control. The materials, methods, and examples are illustrative only and not intended to be limiting.

Overview

Provided herein are compositions and scalable methods of post-processing mycelium materials and/or composite mycelium materials. In some or most embodiments, the mycelium materials and/or composite mycelium materials are post-processed prior to treatment to form preserved mycelium materials.

Exemplary patents and applications discussing methods of growing mycelium include: WIPO Patent Publication No. 1999/024555; G.B. Patent No. 2,148,959; G.B. Patent No. 2,165,865; U.S. Pat. Nos. 5,854,056; 2,850,841; 3,616,246; 9,485,917; 9,879,219; 9,469,838; 9,914,906; 9,555,395; U.S. Patent Publication Nos. 2015/0101509; 2015/0033620 all of which are herein incorporated by reference in their entirety. Additionally, U.S. Patent Publication No. 2018/0282529, filed on Oct. 4, 2018, discusses various mechanisms of solution-based post-processing mycelium material to produce a material that has favorable mechanical characteristics for processing into a textile or leather alternative.

A description of an embodiment with several components in communication with each other does not imply that all such components are required. To the contrary, a variety of optional components may be described to illustrate a wide variety of possible embodiments and in order to more fully illustrate one or more aspects. Similarly, although process steps, method steps, algorithms or the like may be described in sequential order, such processes, methods, and algorithms may generally be configured to work in alternate orders, unless specifically stated to the contrary. In other words, any sequence or order of steps that may be described herein does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more embodiments, and does not imply that the illustrated process is preferred. Also, steps are generally described once per embodiment, but this does not mean they must occur once, or that they may only occur once each time a process, method, or algorithm is carried out or executed. Some steps may be omitted in some embodiments or some occurrences, or some steps may be executed more than once in a given embodiment or occurrence.

Cultivating Mycelium Material

Embodiments of the present disclosure include various compositions of cultivated mycelium materials and methods for production thereof. Depending on the particular embodiment and requirements of the material sought, various known methods of cultivating mycelium may be used. Any fungus that can be cultivated as mycelium may be used. Suitable fungus for use include but are not limited to: Pleurotus ostreatus; Agrocybe brasiliensis; Polyporus squamosus; Rhizopus microspores; Schizophyllum commune; Flammulina velutipes; Hypholoma capnoides; Hypholoma sublaterium; Morchella angusticeps; Macrolepiota procera; Coprinus comatus; Agaricus arvensis; Ganoderma tsugae; Ganoderma sessile and Inonotus obliquus.

In some embodiments, the strain or species of fungus may be bred to produce mycelium with specific characteristics, such as a dense network of hyphae, a highly-branched network of hyphae, hyphal fusion within the network of hyphae, and other characteristics that may alter material properties of the cultivated mycelium material. In some embodiments, the strain or species of fungus may be genetically modified to produce mycelium with specific characteristics.

In most embodiments, the cultivated mycelium material may be grown by first inoculating a solid or liquid substrate with an inoculum of the mycelium from the selected species of fungus. In some embodiments, the substrate is pasteurized or sterilized prior to inoculation to prevent contamination or competition from other organisms. For example, a standard method of cultivating mycelium comprises inoculating a sterilized solid substrate (e.g. grain) with an inoculum of mycelium. Other standard methods of cultivating mycelium comprise inoculating a sterilized liquid medium (e.g. liquid potato dextrose) with an inoculum of mycelium. In some embodiments, the solid and/or liquid substrate will comprise lignocellulose as a carbon source for mycelium. In some embodiments, the solid and/or liquid substrate will contain simple or complex sugars as a carbon source for the mycelium.

In various embodiments, the liquid or solid substrate may be supplemented with one or more different nutritional sources. The nutritional sources may contain lignocellulose, simple sugars (e.g. dextrose, glucose), complex sugars, agar, malt extract, a nitrogen source (e.g. ammonium nitrate, ammonium chloride, amino acids) and other minerals (e.g. magnesium sulfate, phosphate). In some embodiments, one or more of the nutritional sources may be present in lumber waste (e.g. sawdust) and/or agricultural waste (e.g. livestock feces, straw, corn stover).

Once the substrate has been inoculated and, optionally, supplemented with one or more different nutritional sources, the cultivated mycelium material and/or composite mycelium material may be grown in part. In embodiments of producing a composite mycelium material, the inoculated substrate may form part of the composite material, such as particles described in U.S. Pat. No. 9,485,917. In some embodiments, the cultivated mycelium material may be grown through a second material that becomes enmeshed with the mycelium to form a composite material. Various methods of growing networks of cultivated mycelium material that are enmeshed with another material to form a composite material are disclosed in U.S. Pat. No. 9,485,917; U.S. Patent Publication Nos. US2016/0302365 and US2013/0263500, the entirety of which are incorporated herein by reference.

In various embodiments, the cultivated mycelium material may be grown on its own without a second material. In some embodiments, the growth of the cultivated mycelium material will be controlled to prevent the formation of fruiting bodies. Various methods of preventing fruiting body formation as discussed in detail in U.S. Patent Publication No. US 2015/0033620, the entirety of which is incorporated by reference. In other embodiments, the cultivated mycelium material may be grown so that the cultivated mycelium material is devoid of any morphological or structural variations. Depending on the embodiment sought, growing conditions such as exposure to light (e.g. sunlight or a growing lamp), temperature, carbon dioxide may be controlled during growth.

In some embodiments, the cultivated mycelium material may be grown on an agar medium. Nutrients may be added to the agar/water base. Standard agar media commonly used to cultivate mycelium material include, but are not limited to, a fortified version of Malt Extract Agar (MEA), Potato Dextrose Agar (PDA), Oatmeal Agar (OMA), and Dog Food Agar (DFA).

Preserving Mycelium Material

Once the cultivated mycelium material has been grown, it may be separated from the substrate and optionally post-processed in order to prevent further growth by killing the mycelium and otherwise rendering the mycelium imputricible (referred to herein as “preserved mycelium material”). Suitable methods of generating preserved mycelium material can include drying or desiccating the cultivated mycelium material (e.g. pressing the cultivated mycelium material to expel moisture) and/or heat treating the cultivated mycelium material. In a specific embodiment, the cultivated mycelium material is pressed at 190,000 pounds force to 0.25 inch for 30 minutes. In other embodiments, the cultivated mycelium material is pressed to 0.25 inch for 5 minutes. Suitable methods of drying organic matter to render it imputricible are well known in the art. In one specific embodiment, the cultivated mycelium material is dried in an oven at a temperature of 100° F. or higher. In another specific embodiment, the cultivated mycelium material is heat pressed. Various post-processing methods comprising heat and pressure are disclosed in U.S. Patent Publication Nos. 2017/0028600 and 2016/0202365, the entirety of which is incorporated herein by reference.

In some instances, the cultivated mycelium material is treated with one or more agents that are known to transform chitin present in the mycelium into chitosan and/or add functional groups to the chitin in order to generate preserved mycelium material. In various embodiments, the chitin present in the mycelium (or chitin that has been transformed into chitosan) may be treated with an alkaline solution, epoxide reagents, aldehyde reagents, cyclodextrin reagents, graft polymerization, chelating chemistries, carboxymethyl reagents, epoxide reagents, hydroxylalkyl reagents or any combination thereof. Specific examples of these chemistries are disclosed in U.S. Pat. No. 9,555,395, the majority of which is herein incorporated by reference. After functionalization of the chitin, various agents may be used to cross-link chitin. Depending on the functionalization of the chitin group, traditional tanning agents may be used to link functional groups including chromium, vegetable tannins, tanning oils, epoxies, aldehydes and syntans. Due to toxicity and environmental concerns with chromium, other minerals used in tanning such as aluminum, titanium, zirconium, iron and combinations thereof with and without chromium may be used.

In other instances, living or dried cultivated mycelium material is processed using one or more solutions that function to remove waste material and water from the mycelium. In some embodiments, the solutions comprise a solvent such as ethanol, methanol or isopropyl alcohol. In some embodiments, the solutions comprise a salt such as calcium chloride. Depending on the embodiments, the cultivated mycelium material may be submerged in the solution for various durations of time with or without pressure. In some embodiments the cultivated mycelium material may be submerged in several solutions consecutively. In a specific embodiment, the cultivated mycelium material may first be submerged in one or more first solutions comprising an alcohol and a salt, then submerged in a second solution comprising alcohol. In another specific embodiment, the cultivated mycelium material may first be submerged in one or more first solutions comprising an alcohol and a salt, then submerged in a second solution comprising water. After treatment with solution, the cultivated mycelium material may be pressed using a hot or cold process and/or dried using various methods including air drying and/or vacuum drying. U.S. Patent Publication No. 2018/0282529, the entirety of which is herein incorporated by reference, describes these embodiments in detail.

Plasticizing Cultivated Mycelium Material

Various plasticizers may be applied to cultivated mycelium material to alter the mechanical properties of the cultivated mycelium material. U.S. Pat. No. 9,555,395 discusses adding a variety of humectants and plasticization agents. Specifically, the U.S. Pat. No. 9,555,395 discusses using glycerol, sorbitol, triglyceride plasticizers, oils such as linseed oil, drying oils, ionic and/or nonionic glycols. U.S. Patent Publication No. 2018/0282529 further discusses treating the solution-processed mycelium material with plasticizers such as glycerol, sorbitol or another humectant to retain moisture and otherwise enhance the mechanical properties of the cultivated mycelium material such as the elasticity and flexibility of the cultivated mycelium material.

Other similar plasticizers and humectants are well-known in the art, such as polyethylene glycol and fat liquors obtained by emulsifying natural oil with a liquid that is immiscible with oil (e.g. water) such that the micro-droplets of oil may penetrate the material. Various fat liquors contain emulsified oil in water with the addition of other compounds such as ionic and non-ionic emulsifying agents, surfactants, soap, and sulfate. Fat liquors may comprise various types of oil such as mineral, animal and plant-based oils.

Tanning and Dyeing Cultivated Mycelium Material

In various embodiments, it may be ideal to impart color to the cultivated mycelium material. As discussed in U.S. Patent Publication No. 2018/0282529, tannins may be used to impart a color to cultivated mycelium material or preserved mycelium material.

As cultivated mycelium material includes, in part, of chitin, it lacks the functional sites that are abundant in protein-based materials. Therefore, it may be necessary to functionalize the chitin in the cultivated mycelium material in order to create binding sites for acid and direct dyes. Methods of functionalizing chitin are discussed above.

Various dyes may be used to impart color to the cultivated mycelium material such as acid dyes, direct dyes, disperse dyes, sulfur dyes, synthetic dyes, pigments and natural dyes. In some embodiments, the cultivated mycelium material is submerged in an alkaline solution to facilitate dye uptake and penetration into the material prior to application of a dye solution. In some embodiments, the cultivated mycelium material is pre-soaked in ammonium chloride, ammonium hydroxide, and/or formic acid prior to application of a dye solution to facilitate dye uptake and penetration into the material. In some embodiments, tannins may be added to the dye solution. In various embodiments, the cultivated mycelium material may be optionally preserved as discussed above before dye treatment or pre-treatment.

Depending on the embodiment, the dye solution may be applied to the cultivated mycelium material using different application techniques. In some embodiments, the dye solution may be applied to the one or more exterior surfaces of the cultivated mycelium material. In other embodiments, the cultivated mycelium material may be submerged in the dye solution.

In addition to pre-soaking with various solutions, agents may be added to the dye solution to facilitate dye uptake and penetration into the material. In some embodiments, ammonium hydroxide and/or formic acid with an acid or direct dye to facilitate dye uptake and penetration into the material. In some embodiments, an ethyloxylated fatty amine is used to facilitate dye uptake and penetration into the processed material.

In various embodiments, a plasticization agent is added after or during the addition of the dye. In various embodiments, the plasticization agent may be added with the dye solution. In specific embodiments, the plasticization agent may be coconut oil, vegetable glycerin, or a sulfited or sulfated fat liquor.

In some embodiments, the dye solution may be maintained at a basic pH using a base such as ammonium hydroxide. In specific embodiments, the pH will be at least 9, 10, 11 or 12. In some embodiments, the pH of the dye solution will be adjusted to an acidic pH in order to fix the dye using various agents such as formic acid. In specific embodiments, the pH will be adjusted to a pH less than 6, 5, 4 or 3 in order to fix the dye.

In various methods, the cultivated mycelium material and/or preserved mycelium material may be subject to mechanical working or agitation while the dye solution is being applied in order to facilitate dye uptake and penetration into the material. In some embodiments, subjecting the cultivated mycelium material and/or preserved mycelium material to squeezing or other forms of pressure while in a dye solution enhanced dye uptake and penetration. In some embodiments, the cultivated mycelium material may be subject to sonication.

Using the methods described herein, the cultivated mycelium material may be dyed or colored such that the color of the processed mycelium material is substantially uniform. Using the methods described above, the cultivated mycelium material may be dyed or colored such that dye and color is not just present in the surfaces of the cultivated mycelium material but instead penetrated through the surface to the inner core of the processed mycelium material.

In various embodiments, the cultivated mycelium material may be dyed so that the cultivated mycelium material is colorfast. Colorfastness may be measured using various techniques such as ISO 11640:2012: Tests for Color Fastness—Color fastness to cycles of to-and-fro rubbing or ISO 11640:2018 which is an update of ISO 11640:2012. In a specific embodiment, colorfastness will be measured according to the above using a Grey Scale Rating as a metric to determine rub fastness and change to sample. In some embodiments, the mycelium will demonstrate strong colorfastness indicated by a Grey Scale Rating of at least 3, at least 4 or at least 5.

Treating Cultivated Mycelium Material with a Protein Source

In various embodiments, it may be beneficial to treat the cultivated mycelium material with one or more protein sources that are not naturally occurring in the mycelium (i.e. exogenous protein sources). In some embodiments, the one or more proteins are from a species other than a fungal species from which the cultivated mycelium material is generated. In some embodiments, the cultivated mycelium material may be treated with a plant protein source such as pea protein, rice protein, hemp protein and soy protein. In some embodiments, the protein source will be an animal protein such as an insect protein or a mammalian protein. In some embodiments, the protein will be a recombinant protein produced by a micro-organism. In some embodiments, the protein will be a fibrous protein such as silk or collagen. In some embodiments, the protein will be an elastomeric protein such as elastin or resilin. In some embodiments, the protein will have one or more chitin binding domains. Exemplary proteins with chitin binding domains include resilin and various bacterial chitin binding proteins. In some embodiments, the protein will be an engineered or fusion protein comprising one or more chitin binding domains. Depending on the embodiment, the cultivated mycelium material may be preserved as described above before treatment or treated without prior preservation.

In a specific embodiment, the cultivated mycelium material is submerged in a solution comprising the protein source. In a specific embodiment, the solution comprising the protein source is aqueous. In other embodiments, the solution comprising the protein source comprises a buffer such as phosphate buffered saline.

In some embodiments, the solution comprising the protein source will comprise an agent that functions to crosslink the protein source. Depending on the embodiment, various known agents that interact with functional groups of amino acids can be used. In a specific embodiment, the agent that functions to crosslink the protein source is transglutaminase. Other suitable agents that crosslink amino acid functional groups include tyrosinases, genipin, sodium borate, and lactases. In other embodiments, traditional tanning agents may be used to crosslink proteins including chromium, vegetable tannins, tanning oils, epoxies, aldehydes and syntans. As discussed above, due to toxicity and environmental concerns with chromium, other minerals may be used such as aluminum, titanium, zirconium, iron and combinations thereof with and without chromium.

In various embodiments, treatment with a protein source may occur before, after or concurrently with preserving the cultivated mycelium material, plasticizing the cultivated mycelium material and/or dyeing the cultivated mycelium material. In some embodiments, treatment with a protein source may occur before or during preservation of the cultivated mycelium material using a solution comprising alcohol and a salt. In some embodiments, treatment with a protein source occurs before or concurrently with dyeing the cultivated mycelium material. In some of these embodiments, the protein source is dissolved in the dye solution. In a specific embodiment, the protein source will be dissolved in a basic dye solution comprising one or more agents to facilitate dye uptake.

In some embodiments, a plasticizer will be added to the dye solution comprising the dissolved protein source to concurrently plasticize the processed mycelium material. In a specific embodiment, the plasticizer may be a fat liquor. In a specific embodiment, a plasticizer will be added to a protein source that is dissolved in a basic dye solution comprising one or more agents to facilitate dye uptake.

Coating and Finishing Cultivated Mycelium Material

After the cultivated mycelium material has been processed using any combination of plasticization, protein treatment, preserving and tanning as described above, the cultivated mycelium material may be treated with a finishing agent or coating. Various finishing agents common to the leather industry such as proteins in binder solutions, nitrocellulose, synthetic waxes, natural waxes, waxes with protein dispersions, oils, polyurethane, acrylic polymers, acrylic resins, emulsion polymers, water resistant polymers and various combinations thereof may be used. In a specific embodiment, a finishing agent comprising nitrocellulose may be applied to the cultivated mycelium material. In another specific embodiment, a finishing agent comprising conventional polyurethane finish will be applied to the cultivated mycelium material. In various embodiments, one or more finishing agents will be applied to the cultivated mycelium material sequentially. In some instances, the finishing agents will be combined with a dye or pigment. In some instances, the finishing agents will be combined with a handle modifier (i.e. feel modifier or touch) comprising one or more of natural and synthetic waxes, silicone, paraffins, saponified fatty substances, amides of fatty acids, amides esters, stearic amides, emulsions thereof, and any combination of the foregoing. In some instances, the finishing agents will be combined with an antifoam agent.

Mechanically-Working the Material in Solution and After Post-Processing

In various embodiments, the cultivated mycelium material may be mechanically processed in different ways both in solution (i.e. dye solution, protein solution or plasticizer) and after the cultivated mycelium material has been removed from the solution.

While the cultivated mycelium material is in a solution it may be agitated, sonicated, squeezed or pressed to ensure uptake of the solution. The degree of mechanical working will depend on the specific treatment being applied and the level of fragility of the cultivated mycelium material at its stage in processing. Squeezing or pressing of the cultivated mycelium material may be accomplished by hand wringing, mechanical wringing, a platen press, a lino roller or a calendar roller.

Similarly, as discussed above, the cultivated mycelium material may be pressed or otherwise worked to remove solution from the cultivated mycelium material after it is removed from solution. Treating with a solution and pressing the material may be repeated several times.

Once the cultivated mycelium material is fully dried (e.g. using heat, pressing or other desiccation techniques described above), the cultivated mycelium material may be subject to additional mechanical working. Depending on the technique used to treat the cultivated mycelium material and the resultant toughness of the cultivated mycelium material, different types of mechanical working may be applied including but not limited to sanding, brushing, plating, staking, tumbling, vibration and cross-rolling. The cultivated mycelium material may be embossed with any heat source or through the application of chemicals.

In some embodiments, the composite mycelium material may be embossed with any heat source or through the application of chemicals. In some embodiments, the composite mycelium material in solution may be subjected to additional chemical processing, such as, e.g., being maintained at a basic pH using a base such as ammonium hydroxide. In specific embodiments, the pH will be at least 9, 10, 11 or 12. In some embodiments, the pH of the composite mycelium material in solution will be adjusted to an acidic pH in order to fix the composite mycelium material using various agents such as formic acid. In specific embodiments, the pH will be adjusted to a pH less than 6, 5, 4 or 3 in order to fix the composite mycelium material.

Finishing, coating and other steps may be performed after mechanical working or before mechanical working of the dried cultivated mycelium material. Similarly, final pressing steps, including embossing steps, may be performed after or before mechanical working of the dried cultivated mycelium material.

Mechanical Properties of Post-Processed Mycelium Material

Various methods described herein may be combined to provide processed mycelium material that has a variety of mechanical properties.

In various embodiments, the processed mycelium material may have a thickness that is less than 1 inch, less than ½ an inch, less than ¼th inch or less than ⅕th inch. The thickness of the material within a given piece of material may have varying coefficients of variance. In some embodiments, the thickness is substantially uniform to produce a minimal coefficient of variance.

In some embodiments, the processed mycelium material may have an initial modulus of at least 20 MPa, at least 25 MPa, at least 30 MPa, at least 40 MPa, at least 50 MPa, at least 60 MPa, at least 70 MPa, at least 80 MPa, at least 90 MPa, at least 100 MPa, at least 110 MPa, at least 120 MPa, at least 150 MPa, at least 175 MPa, at least 200 MPa, at least 225 MPa, at least 250 MPa, at least 275 MPa, or at least 300 MPa. In some embodiments, the processed mycelium material may have a breaking strength (“ultimate tensile strength”) of at least 1.1 MPa, at least 6.25 MPa, at least 10 MPa, at least 12 MPa, at least 15 MPa, at least 20 MPa, at least 25 MPa, at least 30 MPa, at least 35 MPa, at least 40 MPa, at least 45 MPa, at least 50 MPa. In some embodiments, the processed mycelium material will have an elongation at break of less than 2%, less than 3%, less than 5%, less than 20%, less than 25%, less than 50%, less than 77.6%, or less than 200%. In some embodiments the initial modulus, ultimate tensile strength and elongation at break will be measured using ASTM D2209 or ASTM D638. In a specific embodiment, the initial modulus, ultimate tensile strength and elongation at break will be measured using a modified version ASTM D638 that uses the same sample dimension as ASTM D638 with the strain rate of ASTM D2209.

In some embodiments, the processed mycelium material may have a double stitch tear strength of at least 20 N, at least 40 N, at least 60 N, at least 80 N, at least 100N, at least 120N, at least 140N, at least 160N, at least 180N, or at least 200N. In a specific embodiment, the tongue tear strength will be measured by ASTM D4705.

In some embodiments, the processed mycelium material may have a single stitch tear strength of at least 15N, at least 20N, at least 25N, at least 30N, at least 35N, at least 40N, at least 50N, at least 60N, at least 70N, at least 80N, at least 90N, at least 100N, at least 125N, at least 150N, at least 175N, or at least 200N. In a specific embodiment, the tongue tear strength will be measured by ASTM D4786.

In some embodiments, the processed mycelium material may have a tongue tear strength of at least 1.8N, at least 15N, at least 25N, at least 35N, at least 50N, at least 75N, at least 100N, at least 150N, or at least 200N. In a specific embodiment, the tongue tear strength will be measured by ASTM D4704.

In some embodiments, the processed mycelium material may have a flexural modulus (Flexure) of at least 0.2 MPa, at least 1 MPa, at least 5 MPa, at least 20 MPa, at least 30 MPa, at least 50 MPa, at least 80 MPa, at least lOOMPa, at least 120 MPa, at least 140 MPa, at least 160 MPa, at least 200 MPa, at least 250 MPa, at least 300 MPa, at least 350 MPa, at least 380 MPa. In a specific embodiment, the compression will be measured by ASTM D695.

In various embodiments, the processed mycelium material will have different absorption properties measured as a percentage mass increase after soaking in water. In some embodiments, the % mass increase after soaking in water for 1 hour will be less than 1%, less than 5%, less than 25%, less than 50%, less than 74%, or less than 92%. In a specific embodiment, the % mass increase after soaking in water after 1 hour will be measured using ASTM D6015.

Methods for Production of Cultivated Mycelium Material

Provided herein is a method, comprising: generating a cultivated mycelium material; contacting the cultivated mycelium material with a solution comprising one or more proteins to produce a composition comprising the cultivated mycelium material and one or more proteins, wherein the one or more proteins are from a species other than a fungal species from which the cultivated mycelium material is generated; and pressing the cultivated mycelium material.

In some embodiments, the method includes submerging the cultivated mycelium material in the solution. In some embodiments, the contacting includes contacting the cultivated mycelium material with the solution in a single step.

Exemplary Products Using the Mycelium Material

It is to be appreciated that the above-described growth, treatment, and processing steps applied, in various combinations (including those discussed specifically above and those that may be apparent or derived based on the above description) to mycelium are derived or adapted to produce a material generally resembling leather. To that end, such processing steps can be particularly applied to produce specific mycelium-based materials having characteristics or properties (including tactile, visual, and physical, as described in greater detail herein) similar to those of leather, including leather of various types or having various known properties or attributes. In this manner, mycelium-based material can be cultivated, preserved, plasticized, tanned, dyed, protein-treated, coated, finished, or post-processed according to the processes and variations thereof described herein and in various combinations to produce raw-material that can be manufactured or fabricated into different products typically, or in various forms, being primarily of, or otherwise featuring or including, leather. In certain forms and compositions, this mycelium based material may result in products or articles that meet or exceed consumer, retailer, or manufacturer expectations for similar products of or including leather, including by being amenable or useable in or with the same or similar processing, fabrication, and manufacturing techniques as would be used in working with leather. In other aspects, the manner in which the mycelium material is cultivated and protein-treated, in particular, may allow for fungus breading, modification, or selection, as well as the use or particular liquid and solid substrates, nutritional sources, enmeshed materials, or the like, and proteins for treatment, may allow for controlled production of mycelium with particular properties that offer improved workability or manufacturability over traditional leather, including by way of being suited for additional assembly, fabrication, or finishing techniques. In this manner, such products comprised of, using, or incorporating the various types of mycelium material that may be produced according to or in light of the above description may provide benefits to the consumer and manufacturer beyond what is possible with traditional leather and in addition to the ecological, environmental, and humanitarian benefits that may be realized by substituting the mycelium materials described herein for leather.

Use of Mycelium Material in Footwear

In accordance with the preceding description, in one example, the mycelium material described herein can be used in various types and forms of footwear, including as a substitute for leather, as used in various forms for practically every portion of at least some types of footwear. In various forms, the mycelium material described herein can be used for all or portions of a shoe upper for many types of shoes. In addition, dress shoes and the like typically include insoles made entirely of or including (e.g. on the uppermost, foot-contacting, surface) leather and, in some applications, welts, midsoles and outsoles (including at least the forefoot portion) may also be of leather. In any of these instances, leather can be replaced by specific implementations of the mycelium material described herein having the needed characteristics and accordingly fabricated or manufactured into the desired form. Similarly, either or both of the outsole and upper of various types of slippers may be made from the present mycelium material to, for example, replace leather, and either of all of the upper, outsole, laces, and at least some stitches of moccasins or boat shoes may be made of the present mycelium material.

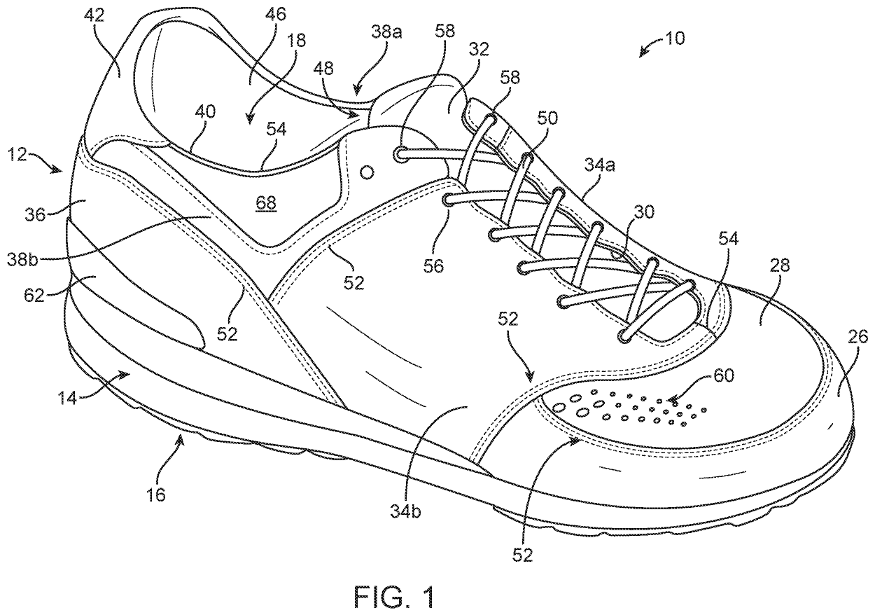

Referring to the embodiment illustrated in FIG. 1, reference numeral 10 generally designates a shoe, particularly in the form of an athletic sneaker. Notably, as discussed herein, the terms “athletic” and “sneaker”, whether used alone or in combination in connection with a particular type or style of footwear does not imply or require that such footwear be strictly used or otherwise useable for any type of athletic activity or for athletics at all. In this respect an article of footwear may simply be of the style or construction of or evoking athletic footwear so as to encompass such footwear, whether used or intended for athletic activity or not (e.g., athleisure or fashion-footwear styled as or similar to athletic sneakers or other variations of athletic footwear, as described below). Further, the descriptions made herein, including in reference to the drawing figures, are merely exemplary with respect to the footwear described and illustrated and that variations may be made to the footwear described herein for purposes of style or fit and/or to make footwear based on the principles and construction described herein suitable for various purposes or conditions. Even further, although construction and production techniques may be discussed herein with respect to particular styles of footwear (e.g. athletic sneakers), such construction and production techniques discussed with respect to one type of footwear may be an acceptable alternative for comparable construction and production techniques discussed herein with respect to other types of footwear (e.g., hiking boots, sandals (including sport sandals) and the like).

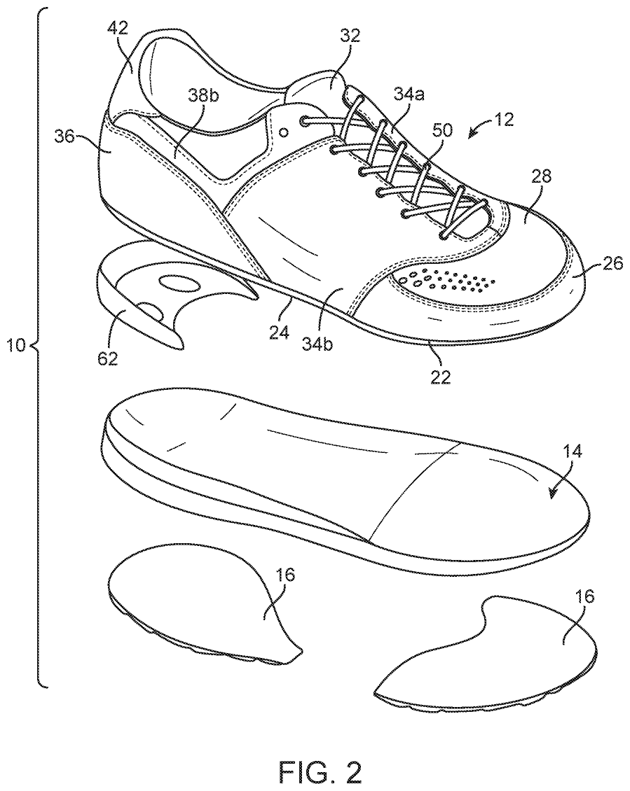

Continuing with reference to FIG. 1, the illustrated athletic sneaker 10 is exemplary of typical construction of athletic sneakers and includes an upper 12, a midsole 14, and an outsole 16, with the upper 12 defining an interior 18 generally suited for receiving the foot of a wearer, and the outsole 16 forming the portion of the athletic sneaker 12 contacting the ground beneath the foot of the wearer. In this respect, the construction of the depicted athletic sneaker 10 is generally typical of other types of footwear with it being noted that the combined midsole 14 and outsole 16 may be collectively referred to as the footwear “outer” and may be used in various forms other than the depicted midsole 14 and outsole 16. In one example, an outer may consist of a midsole material (such as compression-molded ethyl vinyl acetate (“EVA”)) that exhibits both acceptable cushioning and resilience) that at least portions of the ground-contacting surface typically included in a separate outsole may be formed in the midsole material. In a similar manner, an outer can include a rubber outsole 16 can be used alone, without a cushioning midsole for applications of athletic footwear generally referred to as “barefoot” style running shoes or the like. Such variations are considered within the scope of the present disclosure with the depicted examples being varied according to such description. As shown in the example of FIG. 1, the midsole 16 positioned between the upper 12 and the outsole 16 and providing support and cushioning for the sole of the foot, particularly during impact with the ground, as made by the outsole 16. As can be seen in FIG. 2, the interior 18 of the upper 12 is generally enclosed at the lower portion thereof by an lasting board 24 to which the upper 12 is affixed around or adjacent a lower perimeter 22 of the upper 12 (depending on the particular construction method, as discussed further below). The lasting board 24 and or the portions of upper 12 adjacent perimeter 22 are, in turn affixed with midsole 14 with the lasting board 24 being positioned above the midsole 14. As shown in FIG. 3, an insole 24 may be placed within the interior 18 above the lasting board 24. The insole 20 may be at least somewhat cushioned to provide additional comfort to the user and to cover the stitching used to attach the lasting board 24 around the perimeter 22. In one aspect, the insole 20 may also include the mycelium material. This may be done by fabricating the insole 20 entirely from the mycelium material or by covering a foam cushioning layer with a thin layer of the mycelium material such that the uppermost, foot-contacting surface of the insole 20 is of the mycelium material.

As can be seen in FIGS. 1 and 2, the presently described athletic sneaker 10 is exemplary of a sneaker, particularly the upper 12, manufactured using a “cut and sew” process by which the upper 12 is fabricated from a number of individual sections of stock material corresponding with various portions of the upper 12. In particular, the individual sections are cut from the stock material in flat, two-dimensional shapes, as needed as dictated by the desired final form of the upper 12, and are sewn together along various seams that at least partially give the upper 12 its desired three-dimensional form. Such sewing may be augmented by the use of various adhesives along the seams and may be carried in whole or in part over a last that corresponds with the desired shape of the interior 18 of upper 12. In particular, the lasting board 24 is typically sewn to upper 12 over a last and, with respect to typical construction of the depicted athletic sneaker 10, and similar footwear, completed using a “Strobel” stitch using specialized machinery that joins the material portions of the upper 12 that define the perimeter 22 with lasting board 24 in an abutting edge-to-edge seam. The resulting “Strobel sock” including the assembled upper 12 and lasting board 24 is then affixed with the midsole 14, which is most often done using adhesive or the like. In some forms of construction, the affixation between the lasting board 24 and the midsole 14 can be augmented or completed using stitching, such as Blake stitching or the like, or using stitches along particular areas of the upper 12 associated with features attached to the midsole 14, as discussed further below. In general, midsole 14 is of a foam material and may be of multiple different foam materials, including EVA of varying densities or including various inserts, including of plastic and the like. The outsole 16 may be formed of one or more portions of rubber (including various synthetic rubbers and the like) glued, cemented, or otherwise bonded to midsole 14, at least in areas thereof where contact with the ground is made and/or where grip or durability is desired.

With respect to the above-described cut-and-sew fabrication of upper 12, the pieces and sections of upper 12 may generally correspond with particular areas of the upper 12, as discussed above, but may vary according to their particular shape and placement depending on the desired stylistic appearance of the athletic sneaker 10, as well as the desired fit, flexibility, and support of the athletic sneaker 10 (which may be influenced or dictated by the intended use of the athletic sneaker). In the exemplary depiction of FIGS. 1 and 2, the various portions of the upper 12 may include a toe tip 26, and a vamp 28 extending from the toe tip 26 upward to the throat 30 of the athletic sneaker 10. A tongue 32 extends upwardly along the throat 30 from vamp 28, and opposite medial- and lateral-side quarters 34a and 34b extend rearwardly from the toe tip 26, to define the portions of lower perimeter 22 along the respective sides of upper 20, and downwardly away from the throat 30. A heel counter 36 extends around the rear of the upper to connect between the two quarters 34a and 34b around the heel of the wearer. Further, medial and lateral collar portions 38a and 38b can extend upwardly from heel counter 36 and rearwardly from the respective medial and lateral quarters 34 to define respective portions of the topline 40 of the upper 12. A heel tab 42 is positioned above the heel counter and connects between the rearward-most ends of the respective collar portions 38a and 38b to define the rear section of the topline 40. An inner liner 44 (FIG. 3) can extend through all or part of the upper 12 to define the interior 18 thereof and can be affixed with the individual outer portions of the upper 12 along which it extends.

As mentioned above, the shape and configuration of the above-described portions of the upper are exemplary only and can be altered to achieve different appearances, as well as different fit and performance characteristics (flexibility, support, weight, etc.). In one aspect, all or portions of the depicted collar portions 38a and 38b may be integral with the respective quarters 34a and 34b. Still further, the toe tip 26 may be integral with one or both of the quarters 34a and 34b and may be itself formed in one or more portions (e.g. extending separately from respective quarters 34a and 34b), as may vamp 24, which itself may be integral with the toe tip 26. In such construction, additional portions may be assembled with the vamp 24 and or toe tip 26 (e.g., foxing) to cover various seams and/or to provide additional support, protection, or stylistic effect (e.g., a bicycle toe or the like). In still further variations, the collar portions 38a and 38b and/or the heel tab 42 can extend upward relative to the depicted features (or further sections may be added above the existing sections) such that the topline 40 is raised to the level of a mid- or high-top sneaker (i.e. at or above the ankle of the wearer) to provide additional support or protection for the wearer and/or for aesthetic purposes.

As further shown in FIG. 3, additional components may be added between the outer portions of the upper 12 and the liner 44. In particular, a collar lining 46 (which may enclose or be bonded with padding) can be affixed with collar portions 38a and 38b, heel tab 42, and (if applicable) portions of quarters 34a and 34b and can wrap inward over a portion of liner 44 to provide a finished appearance, as well as any padding or grip around the topline 40 that may be beneficial to the wearer. Similarly, additional lining or padding can be added to the inside of tongue 32 to more evenly distribute the force of the laces 50 used to draw together the quarters 34a and 34b to close the throat 30 over the foot.

In accordance with the above, the upper 12 can be made in whole or in part using one or more specific implementations of the above-described mycelium material. As discussed above, the cultivation, preservation, plasticizing, tanning, dyeing, protein-treatment, coating, finishing, and post-processing steps can be individually tailored and collectively combined in various ways to achieve properties particularly suited for use in the depicted and described athletic sneaker 10. In some respects, such properties may allow the mycelium material, as discussed above, to mimic or otherwise meet the expectations for the leather material from which sneakers of the depicted type were originally fabricated and for which the construction and assembly techniques of such sneakers were derived. Notably, in many instances, leather has already been increasingly replaced by other materials, including woven or knitted textile, synthetic leather or suede, various polymeric sheet materials, and combinations of thereof. The use of such materials may provide certain cost advantages over leather (including due to availability), as well as various manufacturing advantages, including the ability to make uppers or portions thereof in a more seamless manner by using material properties or available manufacturing techniques (including, for example so-called three-dimensional weaving or knitting techniques, which may incorporate variations in materials and patterns, as well as shape). Some synthetic materials may also be formable or otherwise adaptable in ways that traditional leathers are not. In other respects, synthetic and textile (including synthetic and natural textile) materials may represent compromises in, or may otherwise reduce, the support or durability of sneakers made from such material compared to those made with leather. Still further, the appearance and tactile qualities of leather may be preferred by consumers in many athletic sneaker (and other footwear) implementations. In this manner, the present mycelium material may be used in place of leather and, further, in place of synthetic materials and textiles (in whole or in part) to address various availability (and in some instances, cost) as well as ecological issues present with respect to leather, as well as preference, support, and durability of synthetic and textile materials when particularly used in fabricating sneakers, including the depicted athletic sneaker 10. This can, in some instances, make the present mycelium material suitable for use in fabricating so-called “retro” sneakers that may evoke or be directly based on particular sneaker designs of traditional leather. Similarly, implementations of the present mycelium material may be used in other applications where the properties of leather are preferred, including for activities where the durability and support of leather are advantageous or where the appearance of leather is also sought.

Accordingly, in one example, the athletic sneaker 10 depicted in FIGS. 1-3 may be such that upper 12 is produced in whole or in primary part of the present mycelium material with the construction and fabrication techniques used in fabricating shoe uppers of or primarily of leather. In this respect, the various portions of upper 12 described above, including toe tip 26, vamp 28, quarters 34a and 34b, heel counter 36, collar portions 38a and 38b, and heel tab 42 (both as depicted in FIGS. 1-3 and as modified for the above-mentioned purposes within the scope of the present disclosure) can be cut in the desired shape from flat-stock mycelium material sheet of the desired composition and sewn together along stitch lines at the interfaces between adjacent portions of the cut material pieces to give upper 12 its desired form. In particular, the cut mycelium material can be joined by topstitching 52 along seams defined by overlapping portions of the material. In the locations of topstitching 52, the raw edges of the mycelium material are generally visible along the respective cutlines of the upper/outermost piece, as is the topstitch 52, which may be doubled or tripled along at least some of the seams for added durability or a decorative effect. It is noted that in areas where additional allowances or tolerances are desired, a felled seam (including lap- or top-felled seams) secured with one or more topstitches can be used. If both the raw edges and seam are to be obscured (or to join adjacent parts in an abutting fashion), stitch and turn seams 54 can be used. As shown, the medial and lateral quarters 34a and 34b can be joined by a stitch and turn seam. Similarly, the collar portions 38a and 38b and heel tab 42 (and optionally, quarters 34a and 34b) can be joined with collar lining 46 by a stitch and turn seam. Still further, tongue 32 can be made from the present mycelium material and can be joined with the lining thereof by stitch and turn seam in which the tongue 32 and tongue lining can be stitched together along the lateral and top edges with the desired outer surfaces facing each other (and, optionally, with any additional padding outside of the liner). The assembled tongue 32 and liner 48 can then be turned over to expose the outside surfaces and to encase the padding before assembly with vamp and/or quarters 34a and 34b (as applicable) using topstitching 52.

In some respects, the properties of the mycelium that are generally comparable to leather can allow the above assembly to be completed using the above techniques with parameters and equipment identical to or comparable to those used in assembly of sneaker uppers of leather, resulting in a similar appearance and the efficiencies of using established techniques and existing machinery. In this manner, the above described pieces of cut mycelium material can have additional processing steps performed thereon, including skivving of edges to reduce the thickness of the material prior to stitching, which can result in a cleaner appearance and easier completion of the stitch and turn seams 54 or any felled stitches incorporated into upper 12. Such skivving can involve pressing or cutting the material at the edge of the desired seam and can be completed using machinery used to skive the edges of leather. In addition to the typical assembly stitching 52 and 54 shown in FIGS. 1-3, embroidery can be applied to the pieces of mycelium material prior to or after assembly of upper 12. In one example, the upper eyelets 56 through which laces pass may benefit from additional enforcement, which can be provided by such embroidery 58 around or through the eyelets 56a. Additional structural embroidery (including to enclose or attach additional structural members, such as strips of metal or plastic) may also be used along quarters 34a and 34b, and decorative embroidery (i.e. stitching not associated with a seam), including for logos or other identifiers or identifying information can be applied elsewhere on upper (including but not limited to on heel tab 42, tongue 32, heel counter 36 and quarters 34a and 34b.

Similarly, the mycelium material may be amenable to other processing and fabrication techniques used for leather that may be useful in fabricating the present athletic sneaker 10. In particular, during or after the above-described tanning process, the mycelium material can be split, removing the portions thereof that are comparable to the “top grain” of leather and resulting in a mycelium material resembling suede and exhibiting comparable tactile and material properties, including a more supple, yet roughened feel and increased flexibility over leather. Similarly, the mycelium material can be sanded, buffed, or stamped to resemble nubuck leather (in appearance and various material characteristics) or can be tanned or dyed with soluble materials to resemble aniline leather. In various examples, the vamp 28, lateral quarter 34a, and collar portions 38a and 38b can be made of a split mycelium material resembling suede to provide increased flexibility and comfort in areas where less support may be needed. similarly, the tongue liner 48 and collar liners 46 may be made of split mycelium material resembling suede to provide increased grip and/or flexibility. In other examples, the plasticization process can be adjusted and applied to split mycelium material (with additional optional embossing) to produce a material similar to bicast leather (or an additional application of polyurethane or vinyl can be applied), which may be used for portions of upper, including the heel counter 36 that may benefit from the additional stiffness provided by such a material.

In one aspect, the above-described processes, by which the presently-used mycelium material is produced, can be tailored to provide the desired characteristics for and resulting from the above-described additional processing. In one example, the mycelium material can be cultivated to provide a structure wherein the “middle” split resembles the tanned hides of the type preferred for fabrication of traditional suede (e.g. lamb, goat, calf, or the like), which may have a tighter fiber network resulting in a less “shaggy” nap on the exposed surface of the resulting material. Such modifications can also be made to result in various different specific leather-like mycelium materials for use in different portions of the upper 12, including more flexible or more rigid materials for the portions discussed above that may utilize or benefit from such properties.

Additionally, the material may be perforated as stock material or after cutting to provide increased flexibility or ventilation in desired areas. The size and shape of perforations 60 may vary among the different portions or may within the particular perforated areas. In one example, the vamp 28 may be perforated by laser cutting after the lateral quarter is cut from the stock material (or during a process by which the vamp 28 and/or other portions of the upper 12 are cut from stock using laser cutting) in an expanding pattern 60 to provide increased flexibility and ventilation in areas where less support or rigidity is needed. Similarly, laser etching may be used to thin (without completely cutting) the mycelium material in various areas or to provide decoration, including by selectively removing the top grain. In an example, the mycelium material may be produced to allow for easier perforation or to provide improved quality of perforation, such as by controlling the networking of the fiber or providing plasticization to reduce material degradation or pilling within the perforations 60 (which can also improve the quality and resilience of the raw edges adjacent topstitching 52). In other examples, the plasticization process can be implemented to provide raw edges, including within perforations, that “self-heal” during laser cutting or are otherwise more amenable to laser cutting or laser etching (e.g., with lower power or less susceptible to burning) compared with leather.

As discussed above, adhesives can be used to improve the strength of the various seams between portions of the upper 12, including both the topstitch seams 52, the stitch and turn seams 54, as well as felled seams, as they may be used in the construction of upper 12. Still further, adhesives may be used alone to affix the combined upper 12 and lasting board 24 to the midsole 14. Solvent-based adhesives (also referred to as cements) have been used for such purposes, including in affixing midsole 14, and are generally accepted as having a relatively low cost and rapid fixing times and high workability. Such solvent-based adhesives and cements can be used with parts or portions of the upper 12 of the presently mycelium material in the same way that they can be used with leather, including to help secure seams of overlapping portions of mycelium material and/or to secure the mycelium forming portions of upper 12 adjacent lower perimeter 22 (or insole 20, which, as discussed above, can also be made from the mycelium material) to midsole 14. In additional aspects, such adhesives can be used to affix the outsole 16 to the midsole 14 or to affix additional elements with upper 12, including the depicted heel stabilizer 62, which is fixed between the rear portions of both the upper 12 and lasting board 24 and the midsole 14.

In some circumstances, ultraviolet (“UV”) light curing or activated adhesives can be used to replace solvent-based adhesives in whole or in part. Such UV curing or UV activated adhesives can include acrylic-based cements or modified epoxy materials. In either case, the compound includes a photoinitiator that undergoes a chemical reaction when exposed to UV light, causing the release of byproducts to that reaction. Those byproducts interact with the remaining compound to cause hardening of the compound or to initiate the reaction that results in hardening. The incorporation of and reliance on the photoinitiator allows for the cement or adhesive to cure “on demand” rather than within a short interval from application (e.g. exposure to air in an acrylic cement or mixing in the case of an epoxy). This may allow for the various portions of upper 12 and/or midsole 14 to be coated along the portions thereof corresponding with seams 52,54 or otherwise for affixation to another element when cut, for example, with the adhesive portions of each piece being activated when ready for affixing with the desired other piece or element. Various heat-activated adhesives can be used in a similar manner. In general, such adhesives can be made to set upon the application of heat above a certain threshold temperature or can use heat as a catalyst for curing (in the case of epoxy, for example). In one example, the heat-activated adhesive can be applied prior to stitching with the assembled upper 12 and/or the assembled athletic sneaker 10 being subsequently run through a heat tunnel to initiate or exacerbate the setting of the adhesive to result in the finished component or product. In some applications, the adhesives can exhibit relatively lower levels of adhesion in an initial state such that pieces or components can be assembled without stitching before heat is applied to set the heat-activated adhesive.

Still further, water-based adhesives and cements have been developed to act as a replacement for solvent-based compounds, as solvents frequently include volatile organic compounds (“VOCs”) or other polluting chemicals (that may also be flammable). In one example, a polyurethane adhesive, for example, may have water as its primary “solvent” in that setting of the adhesive requires that the water evaporate from the compound. Accordingly, the application of heat may be used to speed or cause the adhesive to set. Additionally, pre-heating of the material to be affixed can also help speed the setting process. Water-based adhesives may provide certain characteristics that make them advantageous for the use in shoe fabrication, including fabrication of the present athletic sneaker 10 with the above-described portions being of the present mycelium material. In particular, cross-linking of the compounds during drying may be less affected by ambient humidity (the addition of a hardener can further improve humidity resistance, as well as initial bonding strength, heat resistance, and water decomposition resistance performance. Water based adhesives can exhibit reduced stiffening of the material and may be less prone to interference with stitching. Further, they can be made of a relatively high viscosity to prevent absorption into the materials prior to setting, while still being sufficiently sprayable. Accordingly, in the same manner discussed above, water-based adhesives can be used to help secure the seams 52,54 discussed above and/or to affix additional elements to upper 12 or to fix the upper 12 and lasting board 24 with the midsole 14.

Still further, as shown in FIG. 3, the upper 12 may include additional structural elements in the form of various interfacing elements. In particular, a heel counter interfacing layer 64 can be positioned between heel counter 36 and the underlying portions of quarters 34a and 34b and/or collar portions 38a and 38b. Similarly, the medial and lateral quarters 34a and 34b can include eyelet interfacing 66 along the edges thereof adjacent the throat 30. In both instances, the interfacing 64,66 can be of a relatively rigid textile or a relatively flexible polymeric sheet material, such that the use of the interfacing 64,66 provides additional support for upper 12 in the areas where it is used. In particular, heel counter interfacing 64 (which can be smaller than heel counter 36 to prevent interference with the stitching 52 and to keep interfacing 64 hidden) can provide additional stability for the heel of the wearer. Similarly, the eyelet interfacing can provide additional support for the quarters 34a and 34b in the areas of eyelets 56 to prevent the tightening of laces 50 from damaging the quarters 34a and 34b and/or to allow the eyelets 56 to be positioned closer to the throat 30. The interfacing 64 and 66 may be, at least initially, affixed to the heel counter 36 and the quarters 34a and 34b using adhesives, including any of the adhesives discussed above.

In one respect, the ability to control the material properties of the present mycelium material can also make it more amenable to adhesive than traditional leather, resulting in increased ease of assembly using existing techniques and equipment and fabrication and giving the present athletic sneaker 10, and variants thereof, increased strength and resilience. In various examples, the present mycelium material can be specifically produced to increase surface roughness and decrease overall porosity to improve bonding with various adhesives. Further, adjustments can be made to increase heat resistance and/or heat absorption to allow for higher pre-heating of materials for use with water-based adhesives.

Still further, additional properties of the present mycelium material may provide for the use of additional assembly techniques and may facilitate the implementation of different types of overall construction with different functional and aesthetic characteristics. In one example, the above-described plasticization process can impart a certain degree of thermoplastic properties on the mycelium material. Most notably, the thermoplastic nature of the mycelium material allows it to be molded and bonded using heat. The particular level of such thermoplastic properties exhibited by the material can be controlled by the application of various ones of the plasticization process according to various parameters, as discussed above, as well as the particular characteristics of the cultivation, tanning, and dyeing processes, as these may affect the results of the plasticization process.

In one example, the mycelium material may be produced to be reliably assembled with adhesives such that the stitches 52 and 54 shown in FIGS. 1-3 may be eliminated. This may further be made possible by the thermoplastic nature of the mycelium material, which may facilitate heat-activated bonding between the various portions of upper 12. For example, when using water-based adhesive, the application of heat to the material to promote drying of the adhesive may also cause the pieces of mycelium to fuse together directly. Even further, by using specialized equipment, various portions of the upper 12, or the entirety of upper 12 may be joined together using heat and pressure, without any threaded seams or adhesive. Similarly, portions of upper, such as the upper collar inserts 68 or vamp 28 may be fabricated of textile to add flexibility to upper 12. In one application, various thermoplastic textiles can be used and can be similarly heat bonded to the adjacent portions of upper 12. Heat can also be used in the joining of the assembled upper 12 and insole 20 to the midsole, particularly in applications where insole 20 is of the present mycelium material.

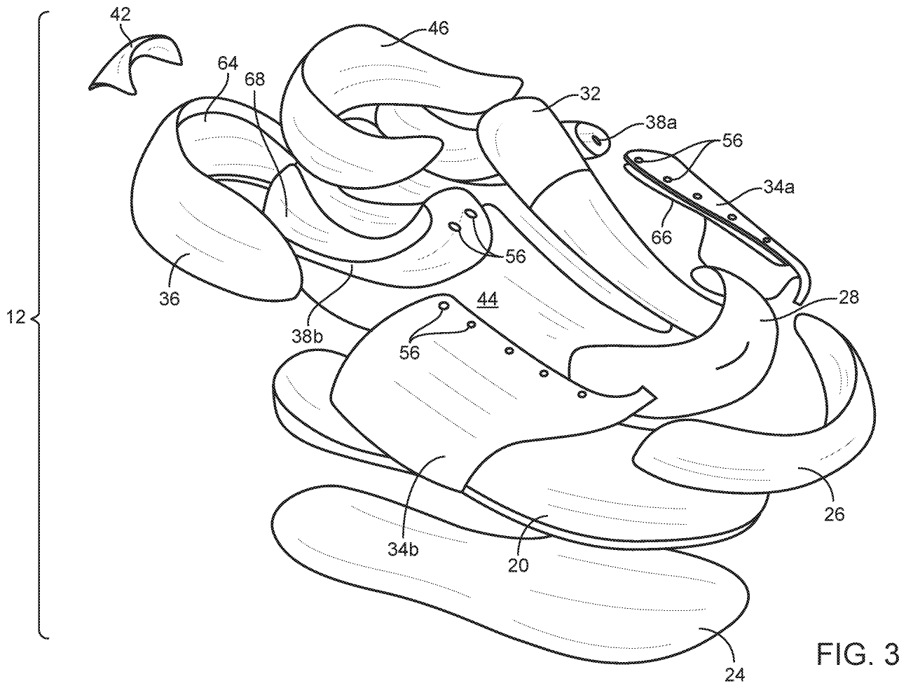

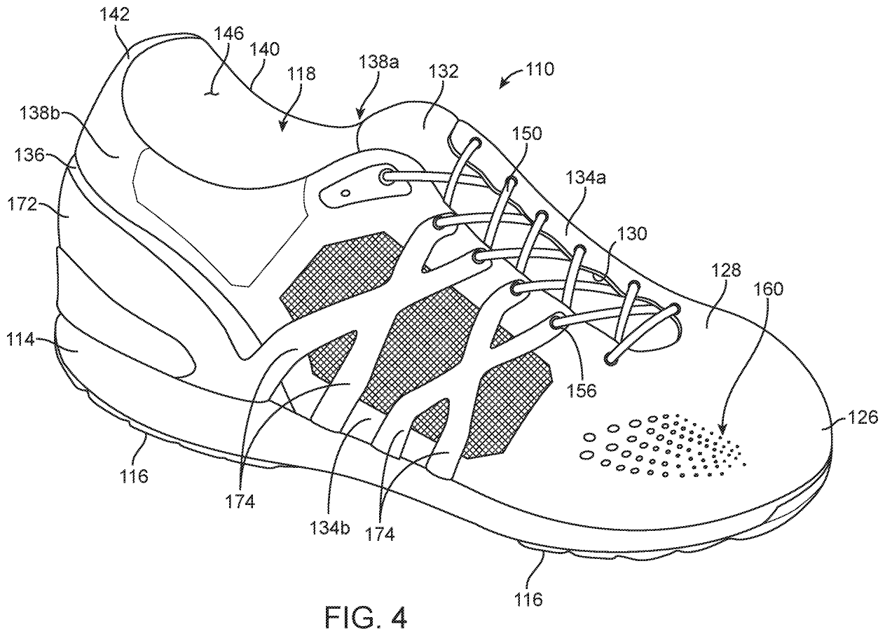

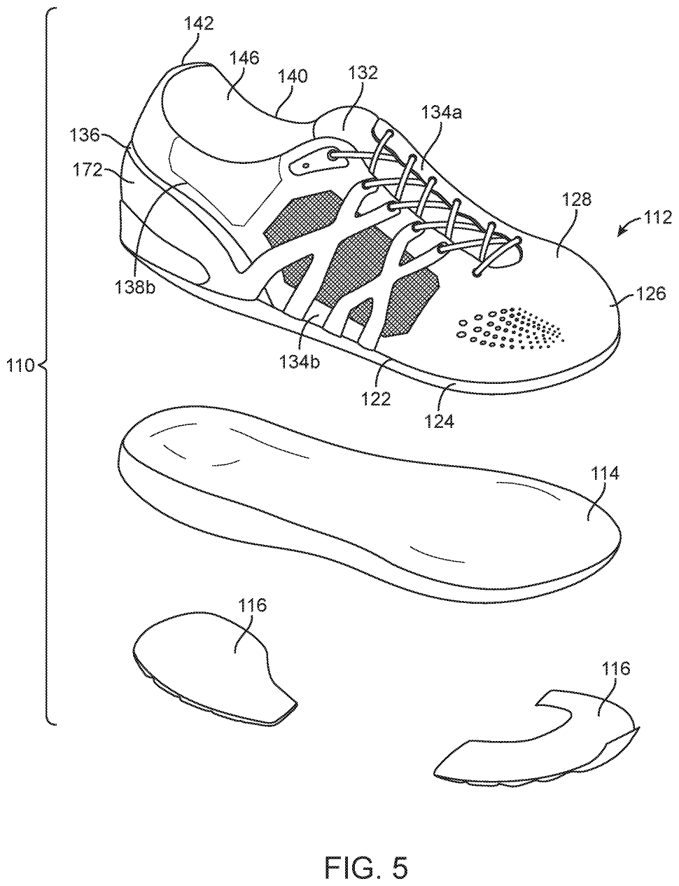

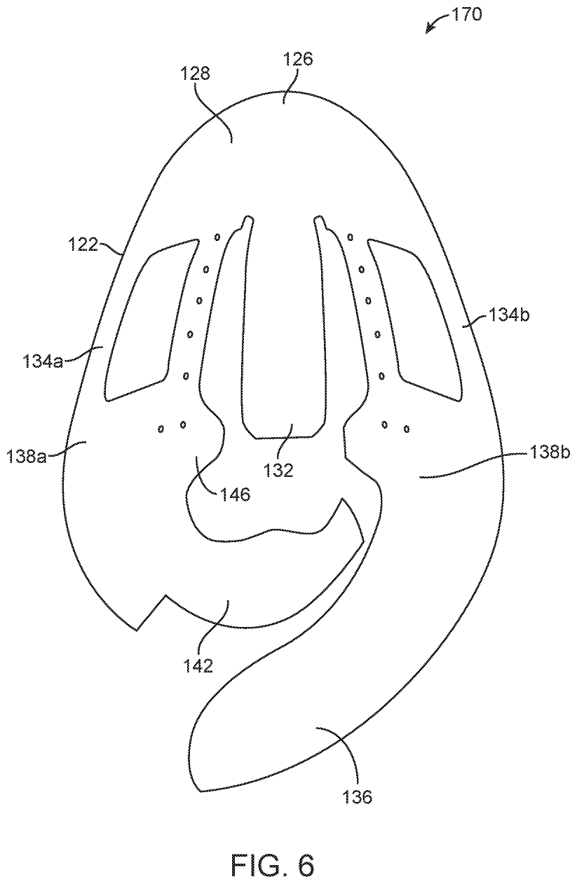

In another example, shown in FIG. 4-6, a variation of the disclosed athletic sneaker 110 can include an upper 112 fabricated from a single piece of mycelium material that can be cut into a shape that includes portions thereof that correspond with the toe tip 126, vamp 128, tongue, 132, quarters 134a and 134b, heel counter 136, and counter portions 138a and 138b. As can be appreciated, cut-and-sew construction, such as of the athletic sneaker 10 discussed above, relies on the construction of seams and the relative placement of the individual parts to impart a three-dimensional shape on the assembled upper 12. Because the single-piece upper 112 illustrated in FIG. 4 is similarly cut from a flat stock of the mycelium material but lacks the seams for relative placement of the parts (with the exception of the joining of the heal counter portion 136 with the collar portion 138a), thermoforming can be used to contribute to the desired three-dimensional form of upper 112. In this manner, the material sheet 170 shown in FIG. 6 can be heated prior to being formed over a last and assembled with the insole 120, with the application of heat making the material sheet 170 pliable such that a three dimensional shape is imparted thereon when formed over the last. The assembled upper 112 and insole 120 can then be bonded with midsole 114 and outsole 116, as shown in FIG. 5. In another example, the material sheet can be loosely formed into the desired shape, including by way of initial affixation of heel counter 136 with collar portion 138a using an adhesive (including the above-described heat-activated adhesives), and placed in a specialized mold where heat may be applied to sheet 170 to allow the pressure from mold to impart the desired three-dimensional form on upper 112.

As further shown, additional features such, as collar lining 146, can be assembled prior to bonding of the upper 112 and insole 120 with midsole, which can be done using adhesives, heat bonding, or traditional stitching. In a variation, collar lining 146 can be of the mycelium material and can be placed in the mold, for example, with the sheet material 170 for direct bonding while upper 112 is shaped. Additional elements, such as an external heel counter reinforcement can be fabricated of an implementation of the present mycelium material and bonded with upper 112 using adhesives and/or heat. In one application, the thermoplastic nature of the mycelium material may facilitate overmolding, including by way of injection molding or the like, of plastic directly onto upper 112. In this manner, a variation of the depicted heel counter reinforcement 172, as well as quarter bands 174 and eyelet reinforcements 178 can be added to upper 112 after formation thereof by an additional step, wherein upper 112 is placed into a subsequent mold with cavities for the heel counter reinforcement 172 and quarter bands 174 such that those features may be formed of a flexible plastic or thermoplastic elastomer material directly onto upper 112. In a further variation, such features can be 3-D printed directly onto upper 112, such as by way of filament deposition, wherein the heat used to extrude the material filament promotes fusion with the mycelium material. In certain aspects, features may be 3-D printed onto the material sheet 170 before additional forming. Alternatively, specifically-adapted equipment can be used to 3-D print features onto the formed upper 112. Additionally, textile portions, such as the counter inserts 176 depicted in FIGS. 3-6 can be assembled with sheet 170, including using adhesives or by heat bonding, as discussed above, when thermoplastic textile is used.

In a further variation, the single sheet 170 of mycelium material may be formed of different particular implementations or types of mycelium material that are bonded together, either in the pre-cut sheet material or after individual sections of the sheet have been separately cut. In one aspect, the material may be bonded in separate layers, such that different outer layers may be bonded over a single inner layer to provide different material properties in the different areas of upper 112 (such as less rigid materials in for the vamp area 128 or within the collar portions 138a and 138b). In this manner, a generally “seamless” upper 112 can be constructed with different sections of mycelium material having different properties or characteristics. Further, additional layers can be added, including waterproofing layers, other lamination, and the like, by a similar process (and can also be done in connection with the material used to form the individual pieces of the upper 12 discussed above). In an example, the collar inserts 168 and collar lining 146 can be included in sheet 170 and can be of a bonded portion of the sheet 170 that exhibits greater flexibility and/or grip.

In either of the above-described embodiments of the athletic sneaker 10 and 110 described herein, various designs, logos, and the like can be added to the sneaker 10,110 using techniques similar to those used in connection with existing sneakers and other footwear. In various examples, the various areas of mycelium material in upper 12 and 112 can be printed, including by pad printing or screen printing. The present mycelium material can also be printed on using a sublimation process in which special ink is printed onto a special sheet and heat pressed onto upper 12 and 112 such that the ink sublimates to penetrate the surface of the mycelium material before returning to a solid state to become a generally permanent part of the mycelium material. Additionally, the thermoplastic nature of the present mycelium material can allow for embossing of graphics or other functional elements using heat and pressure.

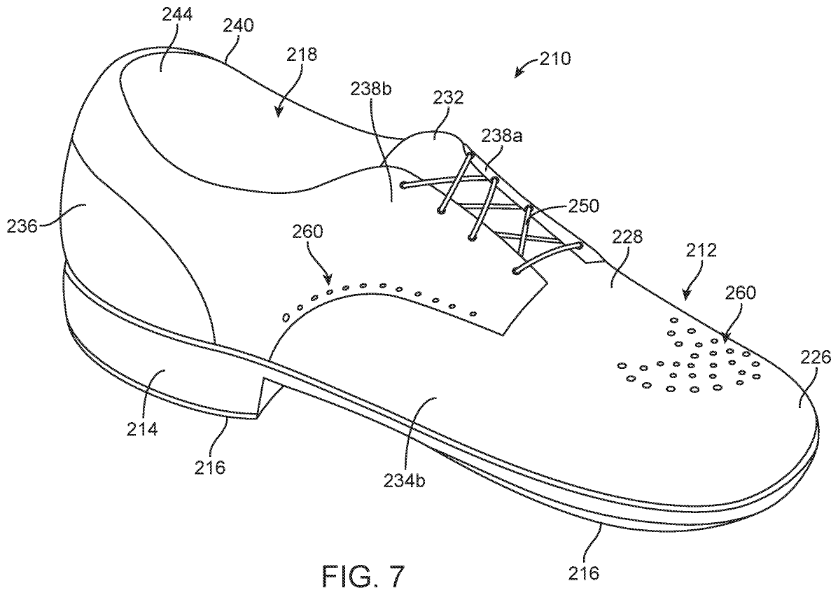

It is to be appreciated that the above techniques and fabrication methods using the mycelium material can also be used to fabricate other types of footwear, including the various types (slippers, sandals, moccasins, boat shoes) mentioned above by using techniques generally similar to those used to make such footwear from leather, while taking advantage of the numerous additional properties of the mycelium material to provide additional benefits for such footwear and the construction thereof according to the principles and variations described above. In this manner, various styles of dress shoes, boots, and the like can also be made of the present mycelium material using various ones of the above-described processes and techniques. In an example, the dress shoe 210 depicted in FIG. 7 can be made of the present mycelium material, which can allow the toe 226 thereof to be formed using heat, rather than requiring leather to be stretched into shape, which can make shoe 210 easier and less costly to manufacture. Additional portions of the depicted dress shoe 210 may be generally similar to the portions of athletic sneakers 10 and 110, as discussed above and are numbered similarly. In a variation, dress shoe 210 or a boot of similar construction can include a single outer, as discussed above, that includes a midsole 214 material suitable for providing a surface that may contact the ground to substitute for the depicted outer 216. In one application, the dress shoe 210 or boot may include a “crepe sole” of crepe rubber or a suitable facsimile or substitute that exhibits a low enough durometer to provide cushioning with the rubber layer being of a thickness comparable to that of a combined midsole and outsole. Other similar applications are also possible.

It will be understood by one having ordinary skill in the art that construction of the described device and other components is not limited to any specific material. Other exemplary embodiments of the device disclosed herein may be formed from a wide variety of materials, unless described otherwise herein.

For purposes of this disclosure, the term “coupled” (in all of its forms, couple, coupling, coupled, etc.) generally means the joining of two components directly or indirectly to one another. Such joining may be stationary in nature or movable in nature. Such joining may be achieved with the two components and any additional intermediate members being integrally formed as a single unitary body with one another or with the two components (e.g., the upper may be coupled to the outsole directly or through the midsole positioned therebetween). Such joining may be permanent in nature or may be removable or releasable in nature unless otherwise stated.

It is also important to note that the construction and arrangement of the elements of the articles, as shown, in the examples above are illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the examples shown in the drawings and described above are merely for illustrative purposes and not intended to limit the scope of the article, which is defined by the following claims as interpreted according to the principles of patent law, including the Doctrine of Equivalents.

### EXAMPLES

### Example 1

### Plasticization of Preserved Mycelium Material

The effect of different methods of preserving material prior to tanning and plasticizing the material was investigated. As a first step, Ganoderma sessile was cultivated to form a substantially homogenous (i.e. devoid of any fruiting bodies or substantial morphological variations) mats of cultivated mycelium material of approximately 21 inches in length by 14 inches in width by 2 inches in thickness. These mats of cultivated mycelium material were then separated from the substrate on which they were grown and treated with two different treatment regimens.

As a first treatment regimen (“Treatment A”), the mats of cultivated mycelium material were submerged in a solution of methanol and 15% by weight calcium chloride (CaCl₂) for 7 days. The solution was then replaced with clean solvent and the mats were then submerged in the same solution for another 7 days. The solution was again replaced with clean solvent and the mats were then submerged in the same solution for another 7 days, for a total of 21 days in solution. The mats of cultivated mycelium material were then pressed to a ½ inch thickness for 5 minutes in a platen press. The mats were then rinsed by submerging the mats in methanol for 3 days and pressed again to a ¼th inch thickness for 30 minutes in a platen press. The mats were then dried in a platen press for 1 day.

As a second treatment regimen (“Treatment B”), the mats of cultivated mycelium material were first pressed to a ¼th inch thickness for 5 minutes in a platen press. The pressed mats were then submerged in a solution of methanol and 15% by weight calcium chloride (CaCl₂) for 14 days. The mats of cultivated mycelium material were then rinsed with submerging the mats in water for 3 days and pressed again to a ¼ inch thickness for 30 minutes in a platen press. The mats of cultivated mycelium material were then dried in a platen press for 1 day.

Mats of cultivated mycelium material that were subject to either treatment were tanned by solution in a solution of tea then plasticized by applying an aqueous solution of 20% by weight glycerin to the mats. The mats of cultivated mycelium material were then pressed in a calendar press to a final width of 0.1 inches and a solution of a 10% by weight non-sulfated fat liquor in water.

To investigate the differences, if any between treatments, various tests were performed on the Treatment A and Treatment B mats. These are tabulated below in Table 1 along with the ASTM standard used to test the material, where applicable. ASTM D638 was modified to set the strain rate to 10 inches per minute. ASTM D6015 was modified to use smaller sample dimensions of 0.25 by 1.0 inches.

TABLE 1
Table 1 - Test Results from Plasticized Preserved Mycelium Material

| | ASTM | Treatment | Average | Standard | Number of |
|---|---|---|---|---|---|
| Metric | standard | type | value | deviation | samples |
| Thickness [mm] | D1813 | Treatment A | 2.556422 | 0.41446 | 161 |
| Density [g/cm³] | None | Treatment A | 0.620259 | 0.131373 | 146 |
| Ultimate tensile | Modified | Treatment A | 2.872381 | 1.26551 | 42 |
| strength [MPa] | D638 | | | | |
| Tensile initial | Modified | Treatment A | 31.96952 | 23.04553 | 42 |
| modulus [MPa] | D638 | | | | |
| Tensile elongation | Modified | Treatment A | 62.37214 | 19.67495 | 42 |
| at break [%] | D638 | | | | |
| Tongue tear max | D4704 | Treatment A | 22.42421 | 6.267487 | 38 |
| force [N] | | | | | |
| Single stitch tear | D4786 | Treatment A | 40.00533 | 9.205338 | 30 |
| max force [N] | | | | | |
| Double stitch tear | D4705 | Treatment A | 62.31533 | 13.58803 | 30 |
| max force [N] | | | | | |
| % absorption in | Modified | Treatment A | 169.4097 | 38.34966 | 59 |
| water [%] | ASTM D6015 | | | | |
| Thickness [mm] | ASTM D1813 | Treatment B | 2.577848 | 0.350316 | 171 |
| Density [g/cm³] | None | Treatment B | 0.621049 | 0.061246 | 151 |
| Ultimate tensile | Modified | Treatment B | 3.694681 | 1.444017 | 47 |
| strength [MPa] | ASTM D638 | | | | |
| Tensile initial | Modified | Treatment B | 39.14064 | 56.30825 | 47 |
| modulus [MPa] | ASTM D638 | | | | |
| Tensile elongation | Modified | Treatment B | 60.8766 | 20.15387 | 47 |
| at break [%] | ASTM D638 | | | | |
| Tongue tear max | ASTM D4704 | Treatment B | 22.90278 | 6.404864 | 36 |
| force [N] | | | | | |
| Single stitch tear | ASTM D4786 | Treatment B | 40.89633 | 16.27867 | 30 |
| max force [N] | | | | | |
| Double stitch tear | ASTM D4705 | Treatment B | 67.625 | 21.56889 | 30 |
| max force [N] | | | | | |
| % absorption in | Modified | Treatment B | 164.4712 | 38.0352 | 75 |
| water [%] | ASTM D6015 | | | | |

### Example 2

### Protein Solution Soaking and Crosslinking

The effect of treating mycelial material with a protein was investigated. As a first step, Ganoderma sessile was cultivated to form a substantially homogenous (i.e. devoid of any fruiting bodies or substantial morphological variations) mats of cultivated mycelium material of approximately 21 inches in length by 14 inches in width by 2 inches in thickness. These mats of cultivated mycelium material were then separated from the substrate on which they were grown.

The mats of cultivated mycelium material were then cut into 5 inch by 5 inch squares and were pressed with a platen press for 5 mins until they were a thickness of ¼th inch. The individual squares of cultivated mycelium material were soaked one of four different solutions for a duration of 1 hour:
1) a solution of 0.5% by weight pea protein in water (“0.5% pea protein in water, no TG”);
2) a solution of 0.5% by weight pea protein in water with approximately 0.25% transglutaminase (“0.5% pea protein in water +TG”);
3) a solution of 0.5% by weight pea protein in phosphate buffered saline with 0.25% transglutaminase (“0.5% pea protein in PBS+TG”);
4) a solution of 0.25% by weight hemp protein in water with 0.25% transglutaminase (“10% hemp protein in water+TG”); and

5) a solution of water with 0.25% transglutaminase (“Water +TG”). After soaking in the protein and transglutaminase solution for 1 hour, the squares of the cultivated mycelium material were pressed again in a platen press to a thickness of ¼ inch for 5 minutes and incubated at 37 degrees Celsius for 16 hours. After incubation the squares of cultivated mycelium material were subject to 62 degrees Celsius for 2 hours in order to deactivate the transglutaminase. The squares were then air dried for 2 days.

To test the efficacy of the transglutaminase, the squares from the same mycelium mat were cut into smaller 0.5 inch by 0.5 inch squares and submerged in water to determine the % mass increase after soaking in water after 1 hour. Table 2 below tabulates the % mass increase for the various types of plant protein treatments.

TABLE 2
Table 2 - % mass increase after soaking water after 1 hour

| | Average | Std Dev | |
|---|---|---|---|
| | % mass | % mass | |
| Solution | increase | increase | Samples |
| 0.5% Rice protein in water + TG | 220 | 34 | 5 |
| 0.5% Rice protein in water + TG | 337 | 67 | 5 |
| 0.25% Hemp protein in water + TG | 133 | 10 | 5 |
| 0.25% Hemp protein in water + TG | 123 | 19 | 5 |
| Water + TG | 119 | 41 | 5 |
| Water + TG | 117 | 35 | 5 |

In order to investigate the effect of the various Table 3 below tabulates the % mass increase after soaking in water for 1hour for the various types of pea protein treatments.

TABLE 3
Table 3 - % mass increase after soaking water after 1 hour

| | Average | Std Dev | |
|---|---|---|---|
| | % mass | % mass | |
| Solution | increase | increase | Samples |
| 0.5% Pea protein in water, no TG | 377 | 14 | 5 |
| 0.5% Pea protein in water + TG | 166 | 30 | 5 |
| 0.5% Pea protein in PBS + TG | 106 | 17 | 5 |
| Repeat 0.5% Pea protein in water + TG | 93 | 10 | 5 |
| Repeat 0.5% Pea protein in water − TG | 74 | 13 | 5 |

### Example 3

### Treatment of Cultivated Mycelium Material with Dye Solution

A variety of different dyeing conditions were used to determine optimal conditions for coloring mats of cultivated mycelium material preserved using Treatment A as described in Example 1. Various combinations of acid and direct dyes were used to evaluate penetration of dye into cultivated mycelium materials under different conditions: direct red dye (DR37), acid green dye (AG68:1), direct black dye (DB168), spirulina blue dye, anthraquinone, natural yellow 3, acid brown dyes (AB425 and AB322) were evaluated for penetration into the cultivated mycelium material.

In various trials, the cultivated mycelium material was first treated with a pre-soak comprising ammonium chloride, with and without a surfactant before the dye solution was applied. In some trials, ammonium hydroxide was added to the dye solution. In some trials, ethyloxylated fatty amine was added to the dye solution. In some trials, formic acid was added to the dye solution. In some trials, oxirane was added to the dye solution. In some trials, sulfated fat liquor was added to the solution. The effect of pH was also studied by adjusting the amount of formic acid and/or ammonium hydroxide in solution.

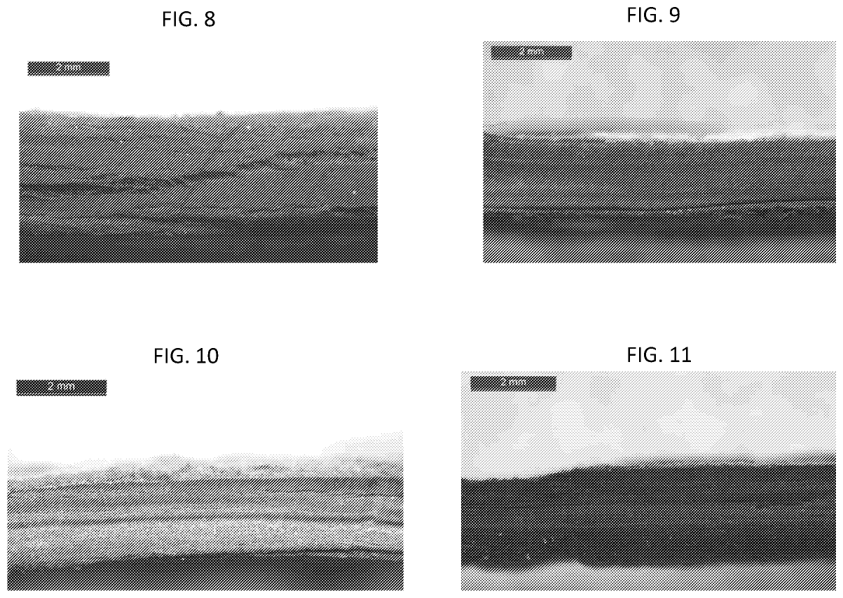

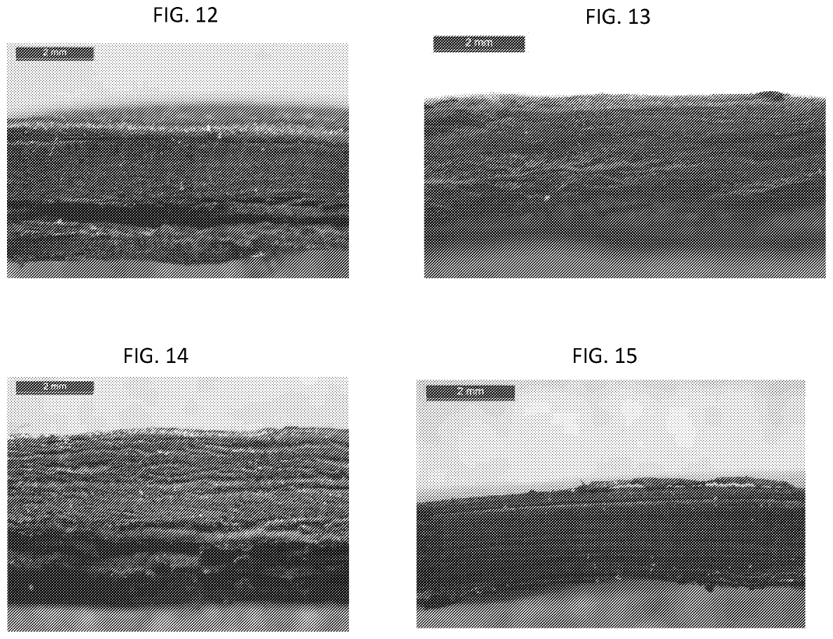

The specific penetration screening trial conditions and results are shown in Table 4 for each of Trials 1, 2, 3, 4, and 5. Corresponding images of dye penetration are shown in FIG. 8, FIG. 9, FIG. 10, FIG. 11 and FIG. 12 as indicated. All images are a cross section of the material taken at 32× magnification. Penetration of dye into the cultivated mycelium material was inspected by microscopy and visually over different time intervals of treatment. The penetration of the dye into the cultivated mycelium material varied over the different experimental conditions and full dye penetration into the mycelium was observed under several experimental conditions. Generally, ammonium hydroxide was observed to facilitate dye penetration and uptake.

TABLE 4
Table 4: Dye Penetration Screening Trials
Trial 1:
Combination of Direct and Acid Dyes

| Penetration | 1.8 g Direct Red 37 (DR37) | |
|---|---|---|
| Screening | 0.6 g Acid Green 68: (AG68:1) | |
| Trial 1 | 35 mL Water | |
| Process: | 0.15 g Mycelium - Sample Ref. 1704 | |
| | (51 g/L DR37, 17.1 g/L AG68:1) | |
| | Time Submerged | Dye Penetration |
| | in Dye Solution | Observations: |
| Dye Penetration | 5 minutes | Not through |
| Observations: | 10 minutes | Not through |
| FIG. 8 | 20 minutes | Not through |
| | 240 minutes | Through |

Trial 2:
Ammonium Chloride (NH₄Cl) Pre-Soak

| Penetration | Pre-soak: | |
|---|---|---|
| Screening | 1.8 g NH₄Cl | |
| Trial 2 | 35 ml Water | |
| Process: | 0.15 g Mycelium - Sample Ref. 1704 | |
| | Leave for 20 minutes | |
| | Dye: | |
| | 1.8 g DR37 | |
| | 0.6 g AG68:1 | |
| | 35 mL Water | |
| | Pre-soaked Mycelium - Sample Ref. 1704 | |
| | Time Submerged | Dye Penetration |
| | in Dye Solution | Observations: |
| Dye Penetration | 5 minutes | Not through |
| Observations: | 10 minutes | Not through |
| FIG. 9 | 20 minutes | Not through |
| | 18 hours | Through |

Trial 3:
NH₄Cl Pre-Soak + Surfactant

| Penetration | Pre-soak: | |
|---|---|---|
| Screening | 1.8 g NH₄Cl | |
| Trial 3 | 35 ml Water | |
| Process: | 0.15 g Mycelium - Sample Ref. 1704 | |
| | Leave for 20 minutes | |
| | Dye: | |
| | 1.8 g DR37 | |
| | 0.6 g AG68:1 | |
| | 35 mL Water | |
| | Pre-soaked Mycelium - Sample Ref. 1704 | |
| | 2 g Oxirane | |
| | Time Submerged | Dye Penetration |
| | in Dye Solution | Observations: |
| Dye Penetration | 5 minutes | Not through |
| Observations: | 10 minutes | Not through |
| FIG. 10 | 20 minutes | Not through |
| | 43 minutes | Through |

Trial 4:
NH₄Cl Pre-Soak + NH₄OH Penetrator in Dye

| Penetration | Pre-soak: | |
|---|---|---|
| Screening | 1.8 g NH₄Cl | |
| Trial 4 | 35 ml Water | |
| Process: | 0.15 g Mycelium - Sample Ref. 1704 | |
| | Leave for 20 minutes | |
| | Dye: | |
| | 1.8 g DR37 | |
| | 0.6 g AG68:1 | |
| | 35 mL Water | |
| | Pre-soaked Mycelium - Sample Ref. 1704 | |
| | 1.8 g NH₄OH | |
| | Time Submerged | Dye Penetration |
| | in Dye Solution | Observations: |
| Dye Penetration | 5 minutes | Not through |
| Observations: | 10 minutes | Not through |
| FIG. 11 | 20 minutes | Not through |
| | 180 minutes | Through |

Trial 5:
Ammonium Hydroxide (NH₄OH) Penetrator + Amine + Dye

| Penetration | 1.8 g DR37 | |
|---|---|---|
| Screening | 0.6 g AG68:1 | |
| Trial 5 | 35 mL Water | |
| Process: | 0.15 g Mycelium - Sample Ref. 1704 | |
| | 1.8 g NH₄OH | |
| | 1.8 g Ethoxylated fatty amine | |
| | Time Submerged | Dye Penetration |
| | in Dye Solution | Observations: |
| Dye Penetration | 5 minutes | Not through |
| Observations: | 10 minutes | Not through |
| FIG. 12 | 20 minutes | Not through |
| | 240 minutes | Through |

The mycelium swelled rapidly upon submersion in the solutions, in particular the ammonia and surfactant related mixtures. Pressure was needed to collapse the structure and remove the dye to produce a mat about 1-2 mm thick. A pressure of 190,000 lbsf was used on mycelial mats approximately 300×450 mm in dimension.

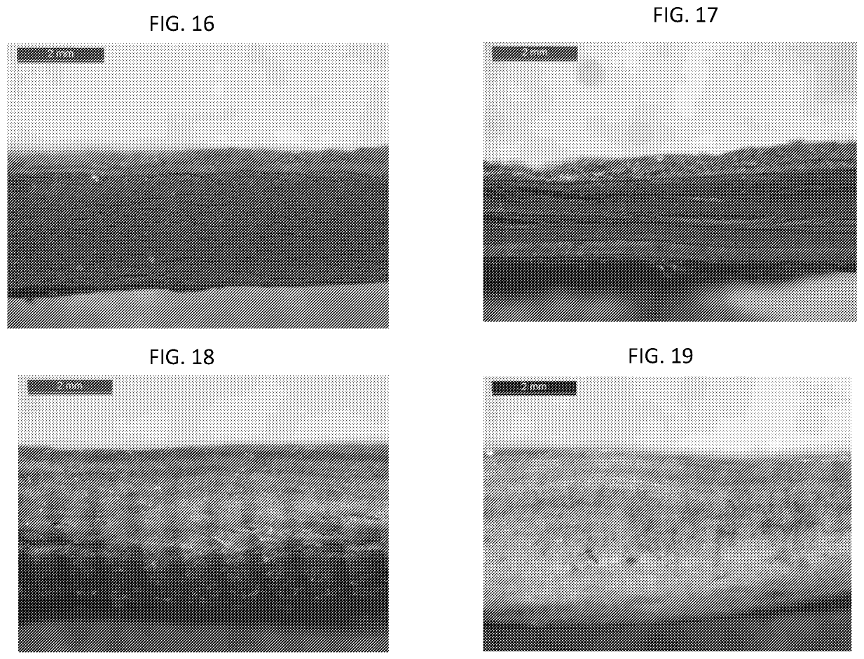

In addition, different substrate samples were dyed using the same combination of direct and acid dyes to assess variations in the dyeing process. The specific substrate screening trial conditions and results are shown in Table 5 for each of Trials 6, 7, 8, 9, and 10. Corresponding images of dye penetration are shown in FIG. 13, FIG. 14, FIG. 15, and FIG. 16 as indicated. All images are cross sections of the material taken at 32× magnification.

TABLE 5
Trial 6:
Combination of Direct and Acid Dye +
Water + NH₄OH - Sample Ref. 1706

| Substrate | 1.8 g DR37 | |
|---|---|---|
| Trial 6 | 0.6 g AG68:1 | |
| Process: | 35 mL Water | |
| | 2 g NH₄OH | |
| | 0.15 g Mycelium - Sample Ref. 1706 | |
| | (51 g/L DR37, 17.1 g/L AG68:1) | |
| | Time Submerged | Dye Penetration |
| | in Dye Solution | Observations: |
| Dye Penetration | 10 minutes | Not through |
| Observations: | 15 minutes | Not through |
| FIG. 13 | 65 minutes | Not through |
| | 18 hours | Not through |
| | 48 hours | Through |

Trial 7:
Combination of Direct and Acid Dye +
Water + NH₄OH - Sample Ref. 1725.1

| Substrate | 1.8 g DR37 | |
|---|---|---|
| Trial 7 | 0.6 g AG68:1 | |
| Process: | 35 mL Water | |
| | 2 g NH₄OH | |
| | 0.15 g Mycelium - Sample Ref. 1725.1 | |
| | (51 g/L DR37, 17.1 g/L AG68:1) | |
| | Time Submerged | Dye Penetration |
| | in Dye Solution | Observations |
| Dye Penetration | 10 minutes | Not through |
| Observations: | 15 minutes | Not through |
| FIG. 14 | 60 minutes | Not through |
| | 180 minutes | 99% through |
| | 18 hours | Through (possibly disintegrating) |

Trial 8:
Combination of Direct and Acid Dye +
Water + NH₄OH - Sample Ref. 1940

| Substrate | 1.8 g DR37 | |
|---|---|---|
| Trial 8 | 0.6 g AG68:1 | |
| Process: | 35 mL Water | |
| | 2 g NH₄OH | |
| | 0.15 g Mycelium - Sample Ref. 1940 | |
| | (51 g/L DR37, 17.1 g/L AG68:1) | |
| | Time Submerged | Dye Penetration |
| | in Dye Solution | Observations |
| Dye Penetration | 10 minutes | Not through |
| Observations: | 15 minutes | Not through |
| FIG. 15 | 60 minutes | Not through |
| | 180 minutes | 99% through |
| | 18 hours | Through |

Trial 9:
Combination of Direct and Acid Dye +
Water + NH₄OH - Sample Ref. 1941

| Substrate | 1.8 g DR37 | |
|---|---|---|
| Trial 9 | 0.6 g AG68:1 | |
| Process: | 35 mL Water | |
| | 2 g NH₄OH | |
| | 0.15 g Mycelium - Sample Ref. 1941 | |
| | (51 g/L DR37, 17.1 g/L AG68:1) | |
| | Time Submerged | Dye Penetration |
| | in Dye Solution | Observations |
| Dye Penetration | 10 minutes | Not through |
| Observations: | 15 minutes | Not through |
| FIG. 16 | 60 minutes | Not through |
| | 18 hours | Through |

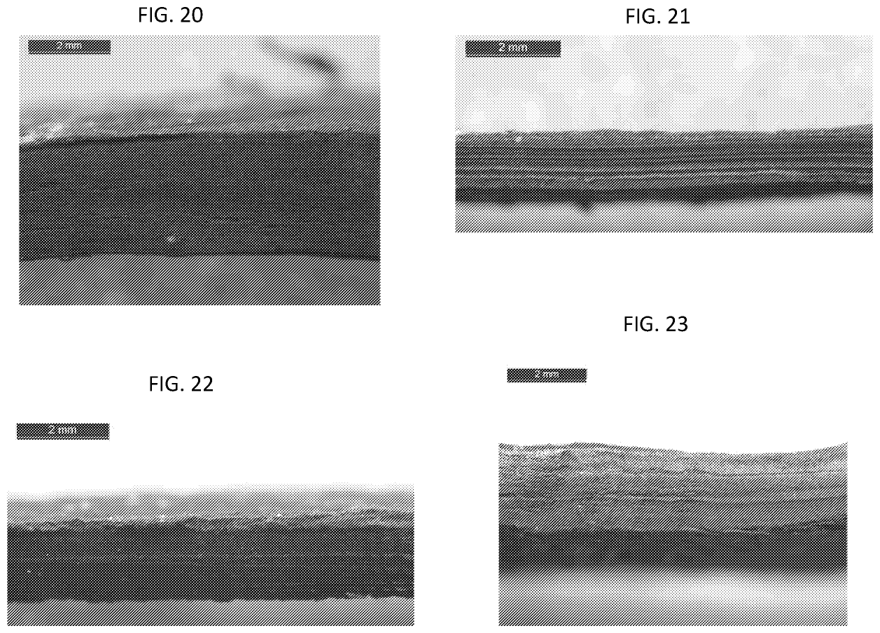

Additional trials were performed using alternative dyes, such as Direct Black 168 (CB168), Spirulina Blue, Natural Yellow 3, Anthraquinone, Acid Brown 322 (AB322), and Acid Brown 425 (AB425). The additional penetration screening trial conditions and results are shown in Table 6 for each of Trials 10, 11, 12, 13, and 14. Corresponding images of dye penetration are shown in FIG. 17, FIG. 18, FIG. 19, FIG. 20, and FIG. 21 as indicated. All images are a cross section of the material taken at 32× magnification.

TABLE 6
Trial 10:
Direct Dyes Only

| Penetration | 1.8 g Direct Black 168 (DB168) | |
|---|---|---|
| Screening | 0.6 g DR37 | |
| Trial 10 | 35 mL Water | |
| Process: | 2 g NH₄OH | |
| | 0.15 g Mycelium - Sample Ref. 1704 | |
| | (51 g/L DB168, 17.1 g/L DR37) | |
| | Time Submerged | Time Submerged |
| | in Dye Solution | in Dye Solution |
| Dye Penetration | 10 minutes | 10 minutes |
| Observations: | 15 minutes | 15 minutes |
| FIG. 17 | 60 minutes | 60 minutes |
| | 18 hours | 18 hours |

Trial 11:
Food Dyes 1.0

| Penetration | 15 g Spirulina Blue | |
|---|---|---|
| Screening | 5 g Anthraquinone | |
| Trial 11 | 35 mL Water | |
| Process: | 0.15 g Mycelium - Sample Ref. 1704 | |
| | Time Submerged | Time Submerged |
| | in Dye Solution | in Dye Solution |
| Dye Penetration | 48 hours | 48 hours |
| Observations: | | |
| FIG. 18 | | |

Trial 12:
Food Dyes 2.0

| Penetration | 10 g Green (Spirulina Blue + Natural Yellow 3) | |
|---|---|---|
| Screening | 7 g Orange (Natural Yellow 3 + Anthraquinone) | |
| Trial 12 | 3 g Spirulina Blue | |
| Process: | 35 mL Water | |
| | 0.15 g Mycelium - Sample Ref. 1704 | |
| | Time Submerged | Time Submerged |
| | in Dye Solution | in Dye Solution |
| Dye Penetration | 48 hours | 48 hours |
| Observations: | | |
| FIG. 19 | | |

Trial 13:
Acid Brown 322

| Penetration | 1.8 g Acid Brown 322 (AB322) | |
|---|---|---|
| Screening | 35 mL Water | |
| Trial 13 | 1.8 g NH₄OH | |
| Process: | 0.15 g Mycelium - Sample Ref. 1704 | |
| | (51 g/L AB322) | |
| | Time Submerged | Time Submerged |
| | in Dye Solution | in Dye Solution |
| Dye Penetration | 18 hours | 18 hours |
| Observations: | | |
| FIG. 20 | | |

Trial 14:
Acid Brown 425

| Penetration | 1.8 g Acid Brown 425 (AB425) | |
|---|---|---|
| Screening | 35 mL Water | |
| Trial 14 | 1.8 g NH₄OH | |
| Process: | 0.15 g Mycelium - Sample Ref. 1704 | |
| | (51 g/L AB425) | |
| | Time Submerged | Time Submerged |
| | in Dye Solution | in Dye Solution |
| Dye Penetration | 18 hours | 18 hours |
| Observations: | | |
| FIG. 21 | | |

These results indicate that the standardized synthetic dyes having a known constitution, higher concentrations, and known penetration performance are able to penetrate the cultivated mycelium material better.

Cultivated mycelium material was incubated with dye with and without agitation to assess the effect of agitation on dye penetration. The agitation trial conditions and results are shown in Table 7 for Trials 15 and 16. Corresponding images of dye penetration are shown in FIG. 22 and FIG. 23 as indicated. All images are a cross section of the material taken at 32× magnification.

TABLE 7
Trial 15:
No Agitation

| Penetration | 1.8 g DR37 | |
|---|---|---|
| Screening | 0.6 g | |
| Trial 15 | AG68:1 35 | |
| Process: | mL Water 2 g | |
| | NH4OH | |
| | 0.15 g Mycelium - Sample Ref. 1940 | |
| | (51 g/L DR378, 17.1 g/L AG68:1) | |
| | Time Submerged | Dye Penetration |
| | in Dye Solution | Observations |
| Dye Penetration | 3 hours | 90% Through |
| Observations: | | |
| FIG. 22 | | |

Trial 16:
With Agitation

| Penetration | 1.8 g DR37 | |
|---|---|---|
| Screening | 0.6 g | |
| Trial 16 | AG68:1 35 | |
| Process: | mL Water 2 g | |
| | NH4OH | |
| | 0.15 g Mycelium - Sample Ref. 1940 | |
| | (51 g/L DR378, 17.1 g/L AG68:1) | |
| | Time Submerged | Dye Penetration |
| | in Dye Solution | Observations |
| Dye Penetration | 3 hours | Through |
| Observations: | | |
| FIG. 23 | | |

Agitation of the cultivated mycelium material aided the uptake and penetration of the dye.

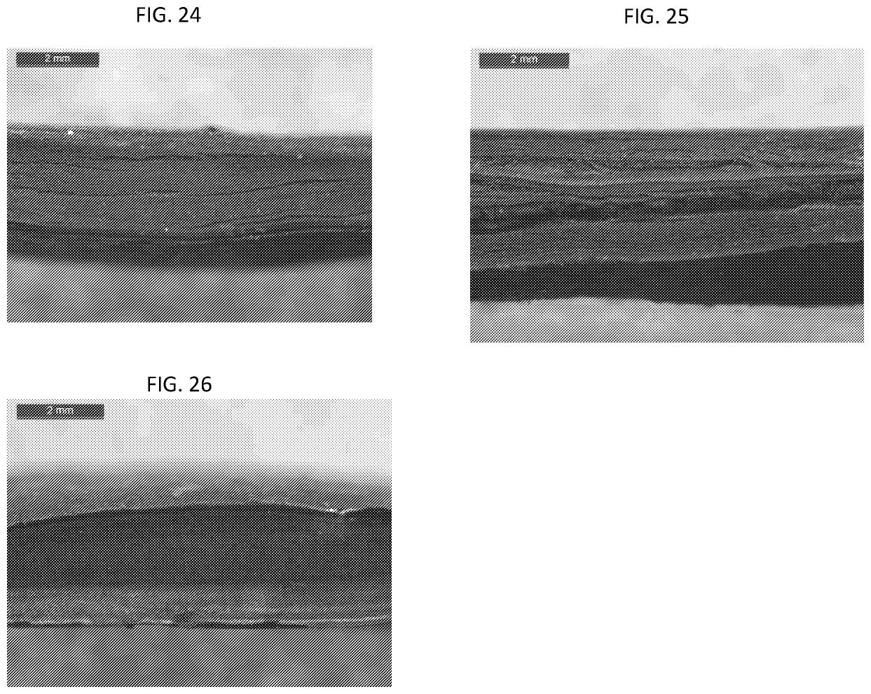

Cultivated mycelium material was incubated with dye at different pH to assess the effect of pH on dye penetration. The agitation trial conditions and results are shown in Table 8 for Trials 17, 18, and 19. Corresponding images of dye penetration are shown in FIG. 24, FIG. 25, and FIG. 26 as indicated. All images are a cross section of the material taken at 32× magnification.

TABLE 8
Trial 17:
pH 7

| Penetration | 1.8 g DR37 | |
|---|---|---|
| Screening | 0.6 g AG68:1 | |
| Trial 17 | 35 mL Water | |
| Process: | 3 g Formic Acid (8.5%) | |
| | 0.15 g Mycelium - Sample Ref. 1940 | |
| | (51 g/L DR378, 17.1 g/L AG68:1) | |
| | Time Submerged | Dye Penetration |
| | in Dye Solution | Observations |
| Dye Penetration | 18 hours | 80% Through |
| Observations: | | |
| FIG. 24 | | |

Trial 18:
pH 8

| Penetration | 1.8 g DR37 | |
|---|---|---|
| Screening | 0.6 g AG68:1 | |
| Trial 18 | 35 mL Water | |
| Process: | 1.8 g Formic Acid (8.5%) | |
| | 0.15 g Mycelium - Sample Ref. 1940 | |
| | (51 g/L DR378, 17.1 g/L AG68:1) | |
| | Time Submerged | Dye Penetration |
| | in Dye Solution | Observations |
| Dye Penetration | 18 hours | 95% Through |
| Observations: | | |
| FIG. 25 | | |

Trial 19:
pH 9

| Penetration | 1.8 g DR37 | |
|---|---|---|
| Screening | 0.6 g AG68:1 | |
| Trial 19 | 35 mL Water | |
| Process: | 0.15 g Mycelium - Sample Ref. 1940 | |
| | (51 g/L DR378, 17.1 g/L AG68:1) | |
| | Time Submerged | Dye Penetration |
| | in Dye Solution | Observations |
| Dye Penetration | 18 hours | Through |
| Observations: | | |
| FIG. 26 | | |

Increasing the pH improved the dye penetration of the cultivated mycelium material.

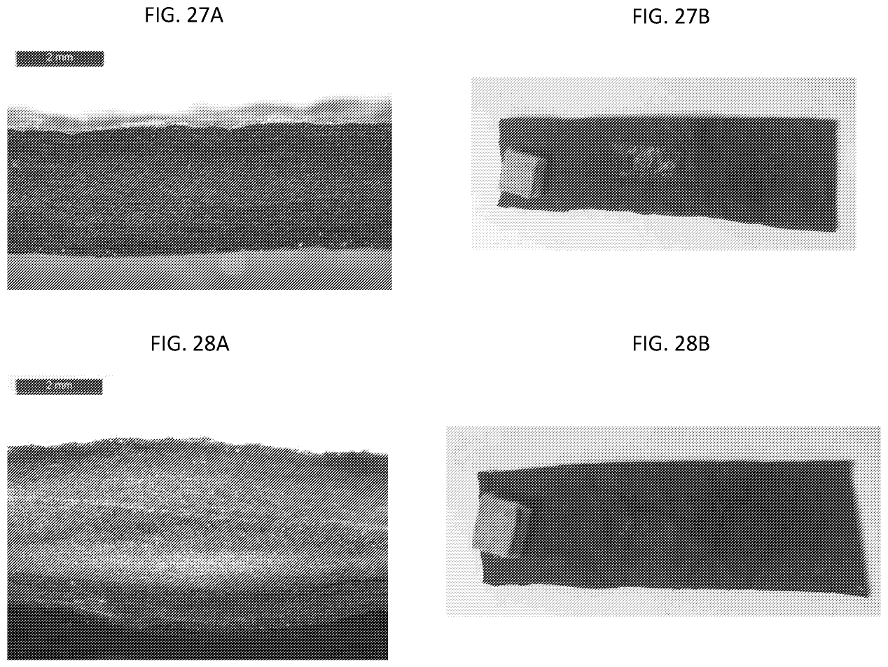

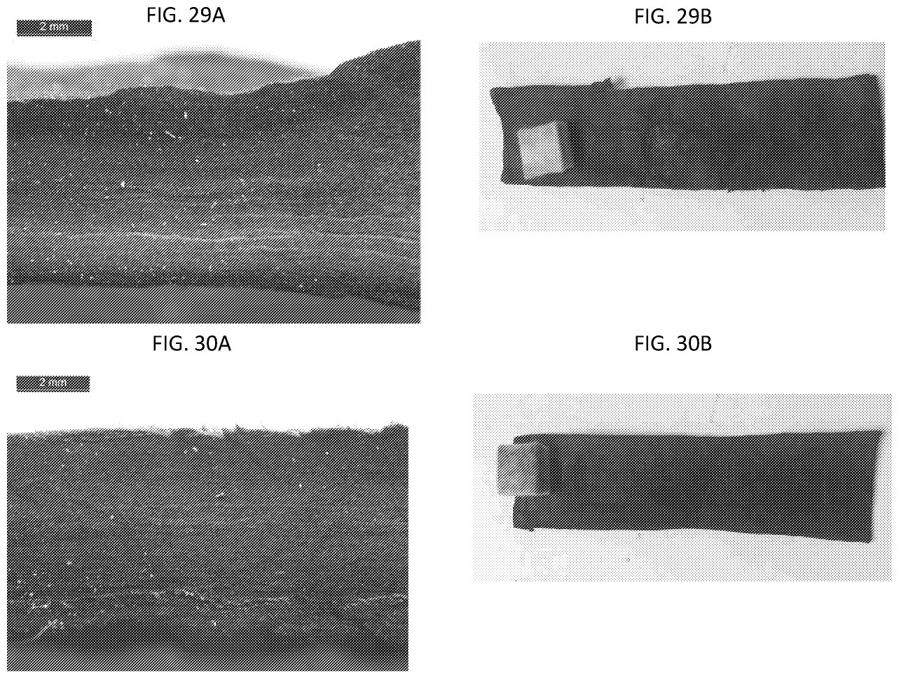

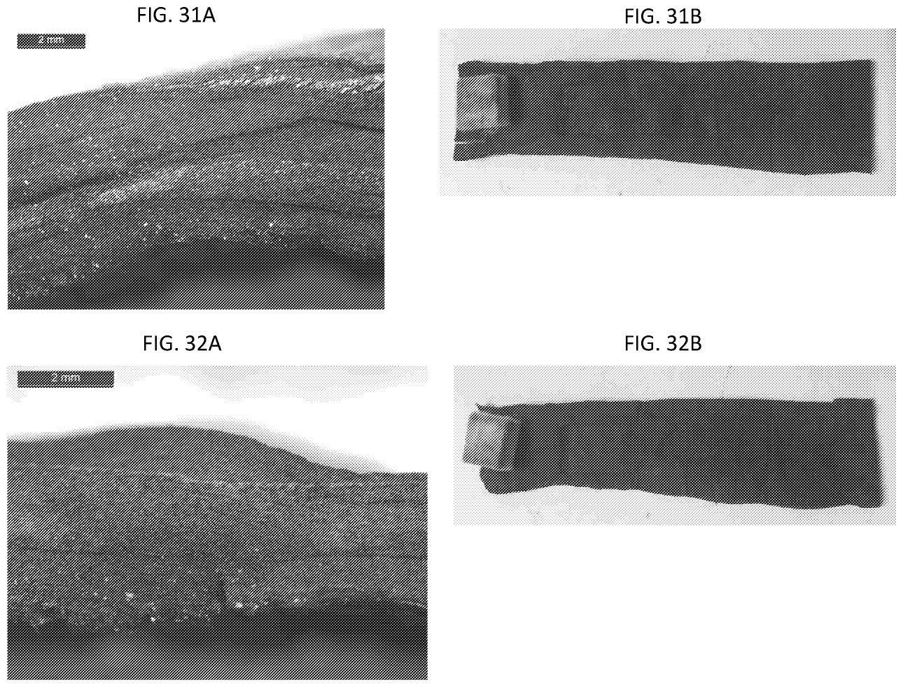

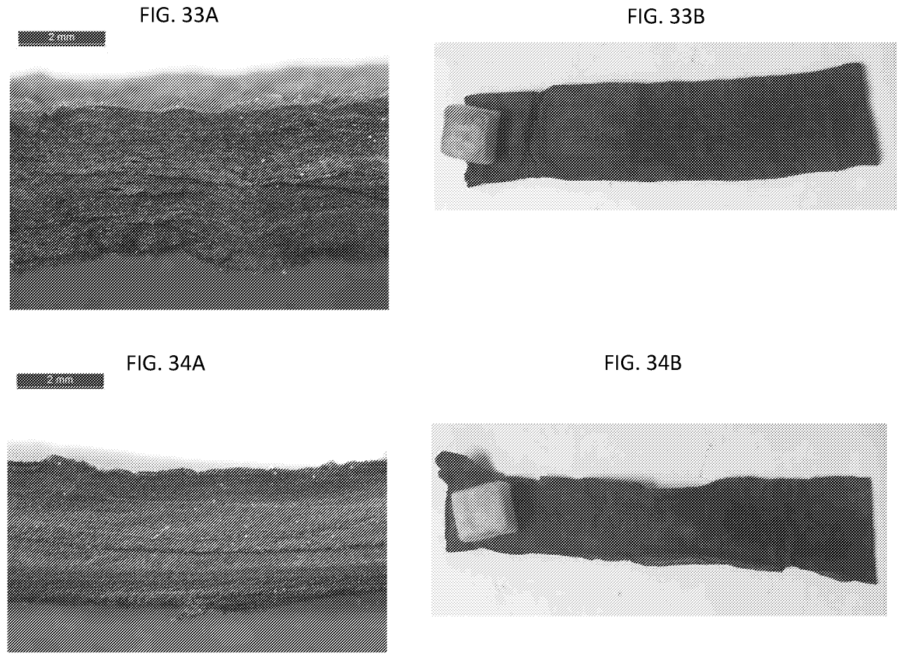

The dye fastness was also assessed via rub tests. The cultivated mycelium material was dyed with various treatments, then rubbed using a Veslic device. Dye fastness was rated after the rub test. The dye fastness trial conditions and results are shown in Table 9 for Trials 20, 21, and 22 using larger amounts of cultivated mycelium material; Trial 23 using additional agitation; Trials 24, 25, and 26, using additional post-dying wash steps; and Trial 27 using a lower dye concentration and post dye wash and squeeze step. Corresponding images of dye penetration are shown in FIG. 27A and 27B, FIG. 28A and 28B, FIG. 29A and 29B, FIG. 30A and 30B, FIG. 31A and 31B, FIG. 32A and 32B, FIG. 33A and 33B, and FIG. 34A and 34B as indicated. All images are a cross section of the material taken at 32× magnification. In each of FIGS. 27-34, the A panel shows the dye penetration and the B panel shows the color fastness.

TABLE 9
Trial 20:
Combination of Direct and Acid Dyes

| Penetration Screening | 18 g DR37 | | | |
|---|---|---|---|---|
| Trial 20 Process: | 6 g AG68:1 | | | |
| FIG. 27A and 27B | 350 mL Water | | | |
| | 15 g NH4OH | | | |
| | 8 g Mycelium - Sample Ref. 1940 (51 g/L DR37, 17.1 g/L AG68:1) Run for 3-4 | | | |
| | hours and agitated | | | |
| | Adjusted pH with Formic Acid (8.5%) until obtained pH 3.5 and left to settle until pH was | | | |
| | pH 4.0 | | | |
| | Compressed with 190,000 lbf for 30 s and dried at ambient overnight | | | |
| Dye Penetration | Struggled to penetrate without ammonia | | | |
| Observations: | After 3-4 hours with agitation not through, extended to overnight | | | |
| | Fixed but lots of dye rinsed out | | | |
| | Uneven dyeing and levelness problems, denser areas have poorer dye uptake | | | |
| | Mycelium dried out, feels rigid and hard | | | |
| Color Fastness: | Veslic Wet Rub (20 cycles) | | | |
| | Grey Scale Rating (GSR) | | | |
| | Pad | | Sample | |
| | 2-3 | Pass | 3-4 | Pass |

Trial 21:
Combination of Direct Dyes

| Penetration Screening | 18 g DB168 | | | |
|---|---|---|---|---|
| Trial 22 Process: | 6 g DR37 | | | |
| FIG. 28A and 28B | 350 mL Water | | | |
| | 15 g NH4OH | | | |
| | 8 g Mycelium - Sample Ref. 1940 (51 g/L DB168, 17.1 g/L DR37) Run for 3-4 hours | | | |
| | and agitated | | | |
| | Adjusted pH with Formic Acid (8.5%) until obtained pH 3.5 and left to settle until pH was | | | |
| | pH 4.0 | | | |
| | Compressed with 190,000 lbf for 30 s and dried at ambient overnight | | | |
| Dye Penetration | After 3-4 hours with agitation not through, extended to overnight | | | |
| Observations: | Not Through | | | |
| | Uneven dyeing and levelness problems, denser areas have poorer dye uptake | | | |
| Color Fastness: | Veslic Wet Rub (20 cycles) | | | |
| | Grey Scale Rating (GSR) | | | |
| | Pad | | Sample | |
| | 2 | Fail | 4 | Pass |

Trial 22:
Dilute Trial - Combination of Direct and Acid Dyes + fatliquor

| Penetration Screening | 6 g DR37 | | | |
|---|---|---|---|---|
| Trial 22 Process: | 2 g AG68:1 | | | |
| FIG. 29A and 29B | 350 mL Water | | | |
| | 15 g NH4OH | | | |
| | 8 g Mycelium - Sample Ref. 1940 (17 g/L DR37, 5.7 g/L AG68:1) 11 g | | | |
| | Sulfated/Sulfited Natural Fatliquor | | | |
| | Run for 3-4 hours and agitated | | | |
| | Adjusted pH with Formic Acid (8.5%) until obtained pH 3.5 and left to settle until pH was | | | |
| | pH 4.0 | | | |
| | Compressed with 190,000 lbf for 30 s and dried at ambient overnight | | | |
| Dye Penetration | After 3-4 hours with agitation not through, extended to overnight | | | |
| Observations: | Through | | | |
| | Uneven dyeing and levelness problems, denser areas have poorer dye uptake | | | |
| Color Fastness: | Veslic Wet Rub (20 cycles) | | | |
| | Grey Scale Rating (GSR) | | | |
| | Pad | | Sample | |
| | 3 | Pass | 4 | Pass |

Trial 23:
Dilute Trial - Combination of Direct and Acid Dyes + fatliquor

| Penetration Screening | 3.5 g DB168 350 mL Water | | | |
|---|---|---|---|---|
| Trial 23 Process: | 3 g NH4OH | | | |
| FIG. 30A and 30B | 3 g Mycelium - Sample Ref. 1940 (10 g/L DB168) | | | |
| | 3.85 g Sulfated/Sulfited Natural Fatliquor Run for 3-4 | | | |
| | hours and agitated | | | |
| | Adjusted pH with Formic Acid (8.5%) until obtained pH 3.5 and left to settle until pH was | | | |
| | pH 4.0 | | | |
| | 3 x Washes (1 x wash = 350 ml H20, 5 mins and tumbled) | | | |
| | Compressed with 190,000 lbf for 30 s and dried at ambient overnight | | | |
| Dye Penetration | Through | | | |
| Observations: | | | | |
| Color Fastness: | Veslic Wet Rub (20 cycles) | | | |
| | Grey Scale Rating (GSR) | | | |
| | Pad | | Sample | |
| | 1-2 | Fail | 4 | Pass |

Trial 24:
3 x Wash Trial

| Penetration Screening | 3.85 g AB425 350 mL Water | | | |
|---|---|---|---|---|
| Trial 24 Process: | 3 g NH4OH | | | |
| FIG. 31A and 31B | 3 g Mycelium - Sample Ref. 1940 (11 g/L AB425) 11 g Sulfated/Sulfited Natural Fatliquor | | | |
| | Adjusted pH with Formic Acid (8.5%) until obtained pH 3.5 and left to settle until pH was | | | |
| | pH 4.0 | | | |
| | 3 x Washes (1 x wash = 350 ml H20, 5 mins and tumbled) Compressed with 190,000 lbf for | | | |
| | 30 s and dried at ambient overnight | | | |
| Dye Penetration | Through | | | |
| Observations: | Uneven dyeing and levelness problems, denser areas have poorer dye uptake | | | |
| Color Fastness: | Veslic Wet Rub (20 cycles) | | | |
| | Grey Scale Rating (GSR) | | | |
| | Pad | | Sample | |
| | 1 | Fail | 4 | Pass |

Trial 25:
5 x Wash Trial

| Penetration Screening | 3.85 g AB425 350 mL Water | | | |
|---|---|---|---|---|
| Trial 25 Process: | 3 g NH4OH | | | |
| FIG. 32A and 32B | 3 g Mycelium - Sample Ref. 1940 (11 g/L AB425) 11 g Sulfated/Sulfited Natural Fatliquor | | | |
| | Adjusted pH with Formic Acid (8.5%) until obtained pH 3.5 and left to settle until pH was | | | |
| | pH 4.0 | | | |
| | 5 x Washes (1 x wash = 350 ml H20, 5 mins and tumbled) Compressed with 190,000 lbf for | | | |
| | 30 s and dried at ambient overnight | | | |
| Dye Penetration | Through | | | |
| Observations: | Uneven dyeing and levelness problems, denser areas have poorer dye uptake | | | |
| Color Fastness: | Veslic Wet Rub (20 cycles) | | | |
| | Grey Scale Rating (GSR) | | | |
| | Pad | | Sample | |
| | 1 in patches | Fail | 4 | Pass |

Trial 26:
7 x Wash Trial

| Penetration Screening | 3.85 g AB425 350 mL Water | | | |
|---|---|---|---|---|
| Trial 26 Process: | 3 g NH4OH | | | |
| FIG. 33A and 33B | 3 g Mycelium - Sample Ref. 1940 (11 g/L AB425) 11 g Sulfated/Sulfited Natural Fatliquor | | | |
| | Adjusted pH with Formic Acid (8.5%) until obtained pH 3.5 and left to settle until pH was | | | |
| | pH 4.0 | | | |
| | 7 x Washes (1 x wash = 350 ml H20, 5 mins and tumbled) Compressed with 190,000 lbf for | | | |
| | 30 s and dried at ambient overnight | | | |
| Dye Penetration | Through | | | |
| Observations: | Uneven dyeing and levelness problems, denser areas have poorer dye uptake | | | |
| Color Fastness: | Veslic Wet Rub (20 cycles) | | | |
| | Grey Scale Rating (GSR) | | | |
| | Pad | | Sample | |
| | 2-3 | Pass | 3-4 | Pass |

Trial 27:
Lower Dye Concentration and “Squeeze” Trial

| Penetration Screening | 3.5 g DB168 300 mL Water | | | |
|---|---|---|---|---|
| Trial 27 Process: | 3 g NH4OH | | | |
| FIG. 34A and 34B | 3 g Oxirane | | | |
| | 3 g Ethoxylated fatty amine | | | |
| | 3 g Mycelium - Sample Ref. 1940 (12 g/L DB168) | | | |
| | Manual penetration by hand squeezing (approximately 10 minutes) Adjusted pH with Formic | | | |
| | Acid (8.5%) | | | |
| | Obtained pH 3.0, target was pH 4.0 (approximately 17 g) | | | |
| | Washed and squeezed approximately 7 times (until no dye released) Left to stand at ambient | | | |
| | for 3 hours | | | |
| | Compressed with 190,000 lbf for 30 s and dried at ambient overnight | | | |
| Dye Penetration | Through - good and relatively rapid uptake with hand squeezing | | | |
| Observations: | | | | |
| Color Fastness: | Veslic Wet Rub (20 cycles) | | | |
| | Grey Scale Rating (GSR) | | | |
| | Pad | | Sample | |
| | 3 | Pass | 4 | Pass |

Trial 27 indicates that a squeezing action enabled a rapid uptake of dye in comparison to gentle agitation. When the dyed mycelium material was placed in water after the dye treatment the material did not release the dye. Instead, pressure was required to release the dye from the mycelium material after dyeing

These results indicate that the use of ammonia aided in dye penetration and that an alkaline pH provided better dye penetration.

### Example 4

### Treatment of Cultivated Mycelium Material with Dye Solution, Protein Solution and Plasticizers

Mats of cultivated mycelium material preserved using Treatment A as described in Example 1 were treated with a number of different dyeing solutions combined with plant proteins (soy protein and pea protein) to determine the effect of protein treatment on dyeing cultivated mycelium material. Briefly, 5.5 g or 11 g of Protein (soya or pea—supplier of both Pulsin) was added to 500 ml of water and sonicated at 40° C. for 60 min. Mycelium material samples were cut to 150 mm×35 mm and incubated in the protein solution. While in the protein solution, the mycelium materials were rolled (squeezed) with a lino-roller 5 times, incubated for 15 min, and rolled an additional 5 times, before being left to soak for an additional 60 min. For dying, 2.5 g of Acid Brown 425 (BASF) was added to 500 ml water at 50° C. and the pH adjusted to 10 using ammonia solution. In some trials, a plasticizer was added to the dye solution. The samples were removed from the protein solution and placed into the dye solution. The samples were rolled 15 times, incubated for 15 min, and rolled an additional 15 times on the reverse. The samples were incubated in the dye solution overnight. Excess dye was removed by washing with water and gently squeezing for approximately 5 min. The samples were allowed to dry at room temperature. For all trials a wet rub fastness test was performed using the BS EN ISO 11640:2012 protocol to test for color fastness in leather. 20 cycles of wet rub were performed and rated using the Grey Scale Rating (GSR) system.

In most trials, the dye solution was kept at a basic pH (pH 10) during dyeing and the pH was then reduced to an acid pH (pH 4-6) to fix the dye. In some trials, a plasticizer such as fat liquor (e.g. Trupon ® AMC and DXV from Trumpler), vegetable glycerin or coconut oil was added to dye solution. In some trials a lino-roller was used to squeeze the cultivated mycelium material in a protein solution and/or a dye solution. Control samples without plasticizer shower poor flexibility. Various amounts of protein were used and excessive protein was shown to generate undesirable results. In some trials, fungicides were added to the dye solution.

In some trials, tannins were used in combination with various dyes to treat the cultivated mycelium material, with and without the addition of protein. In some trials, plasticization steps occurred after dyeing steps and fungicide was added to the plasticizer.

In addition to visual inspection of dye penetration, the hand feel of the samples was evaluated for softness and flexibility. An even distribution of dye (or color) over the surface of the cultivated mycelium material was observed over several experimental conditions. In some conditions, dye penetration through the cultivated mycelium material was observed. Many of the conditions produced material that was soft and flexible. Some samples were evaluated by appearance and the rub fastness of the dye was evaluated by staining and change to a sample using the Grey Scale Rating (GSR) as a metric. In some trials, a biocide was added to the dye solution.

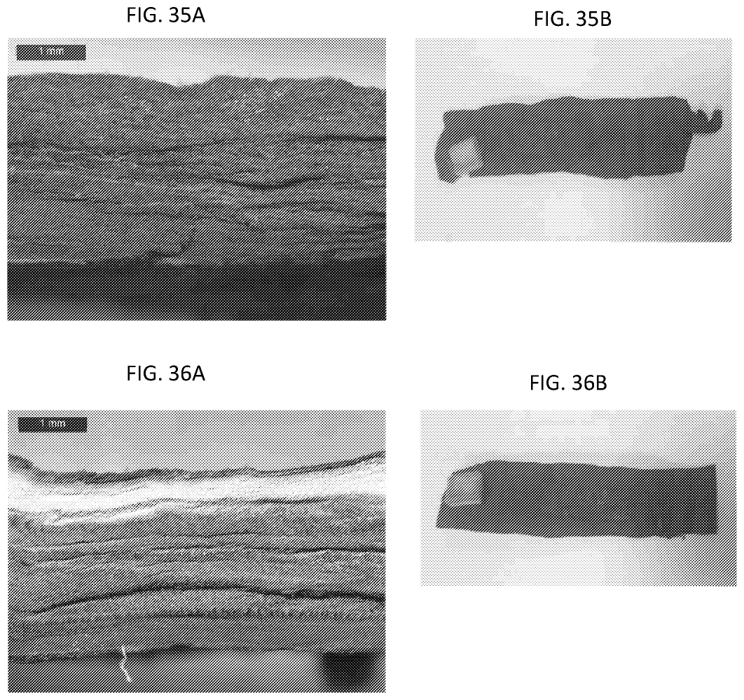

Results from these trials are included as Tables 10-16 and FIGS. 35A and 35B to FIGS. 54A and 54B as indicated. All cross section microscope images were taken at 25.6 magnification. For each trial, the dyed mycelium material cross section is shown in panel A and the rub fastness is shown in panel B.

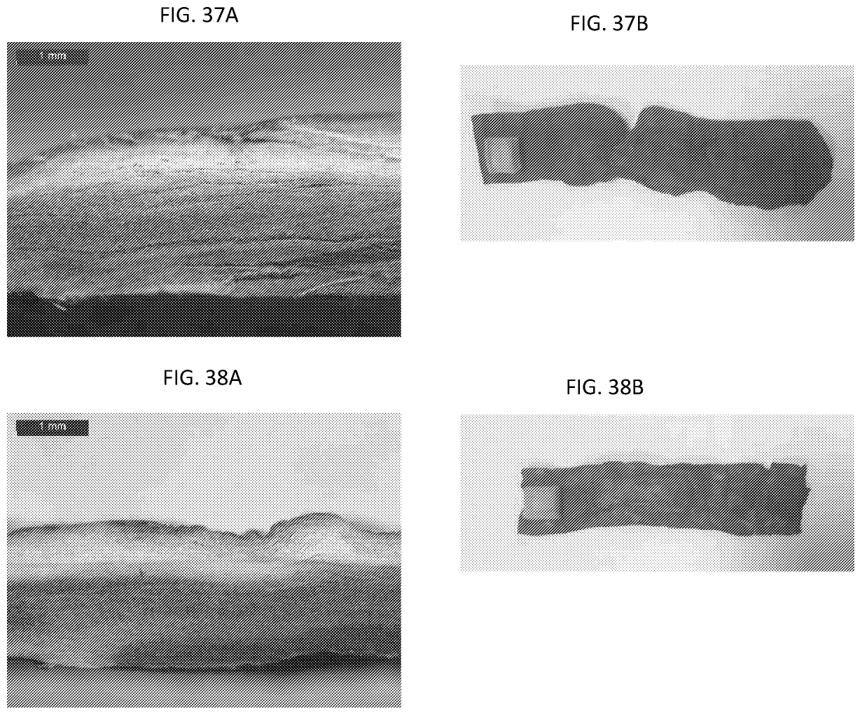

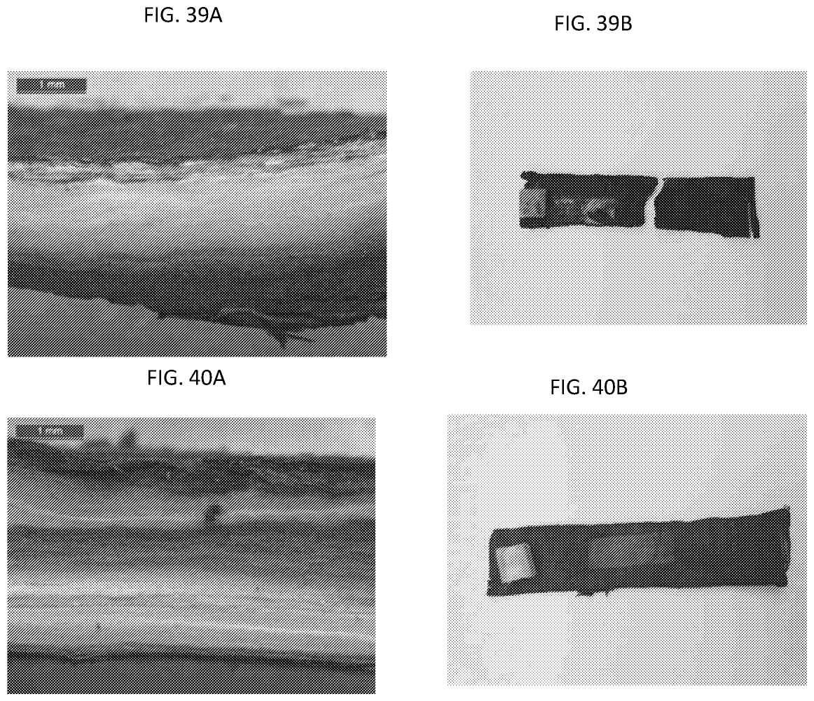

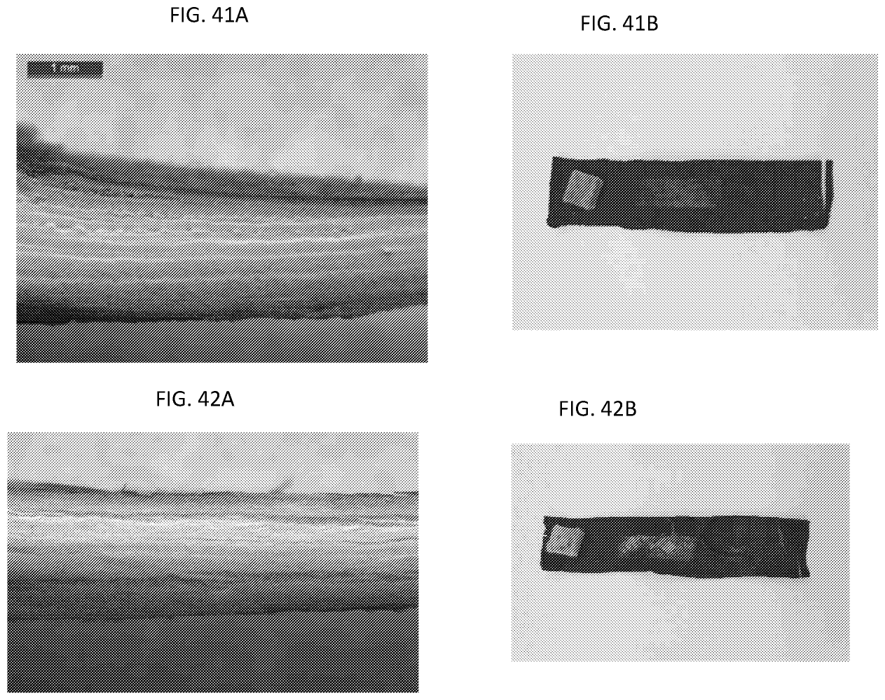

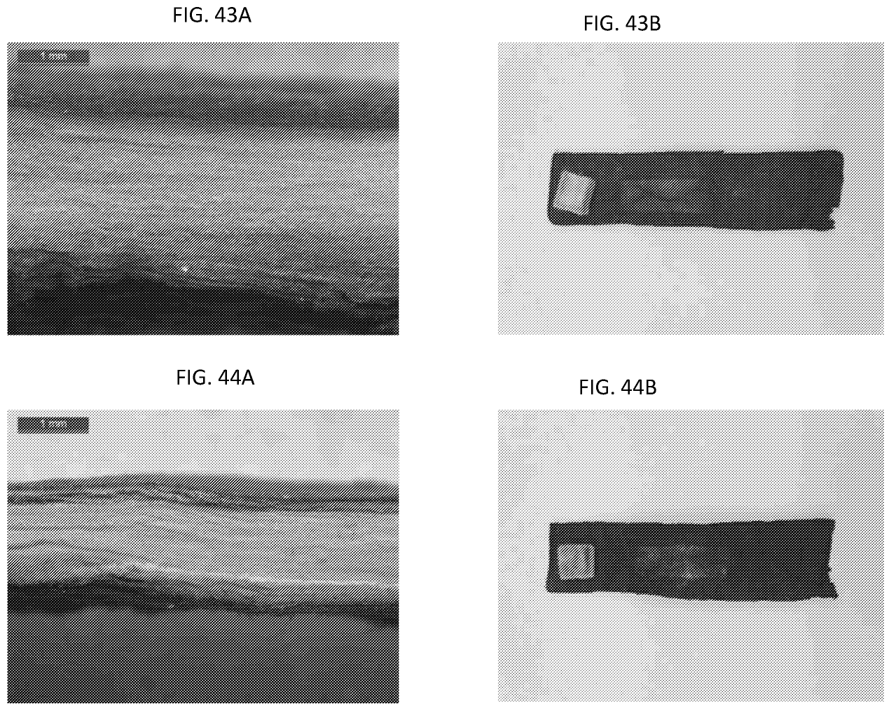

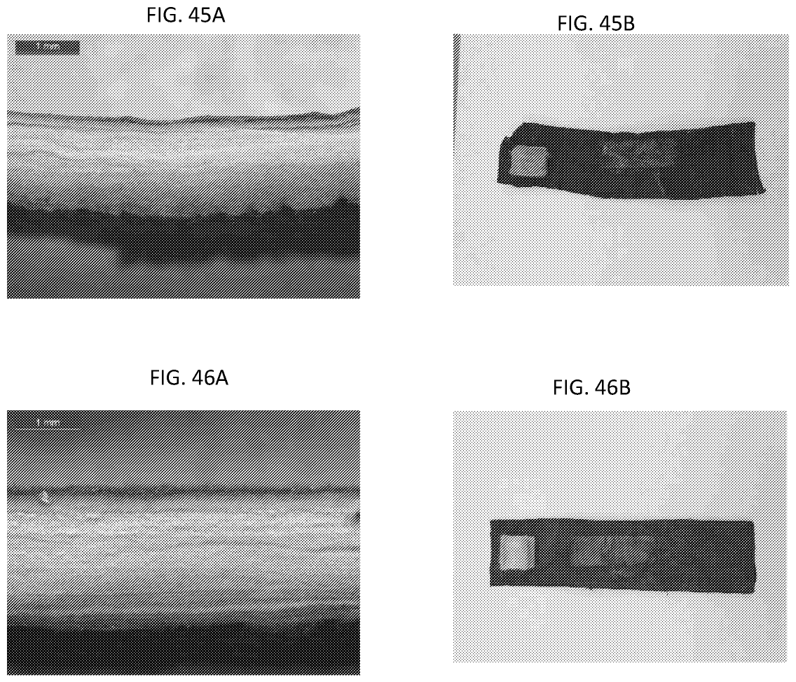

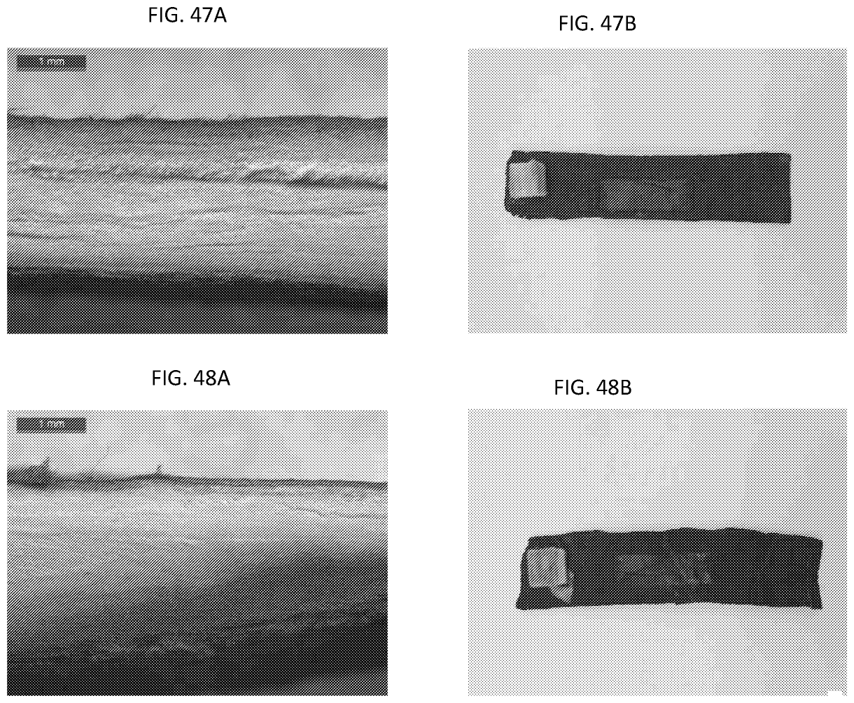

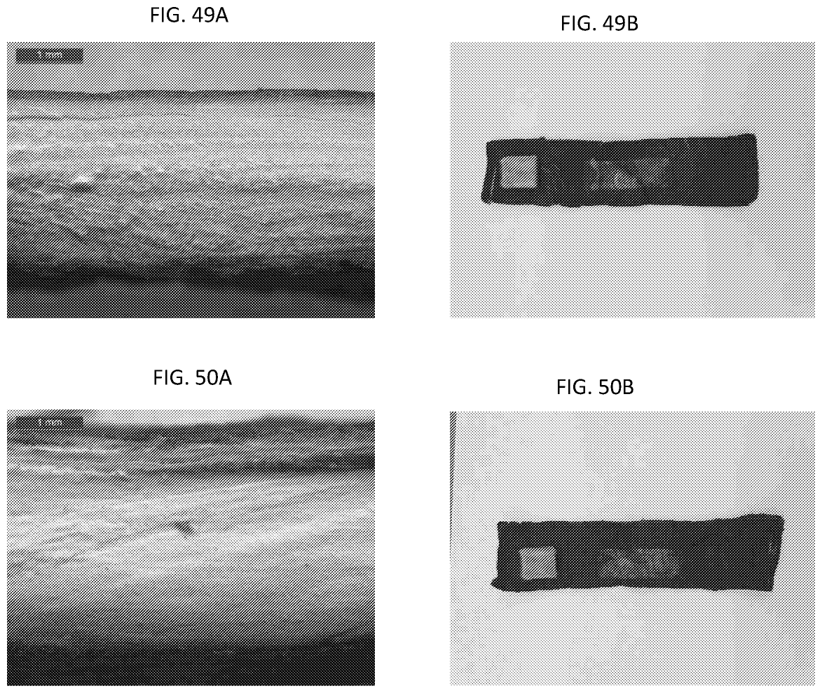

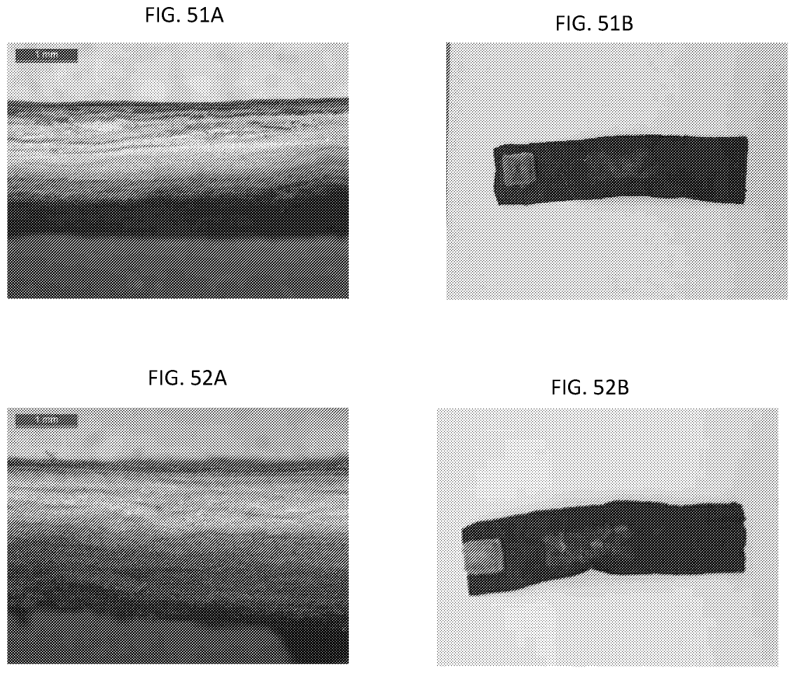

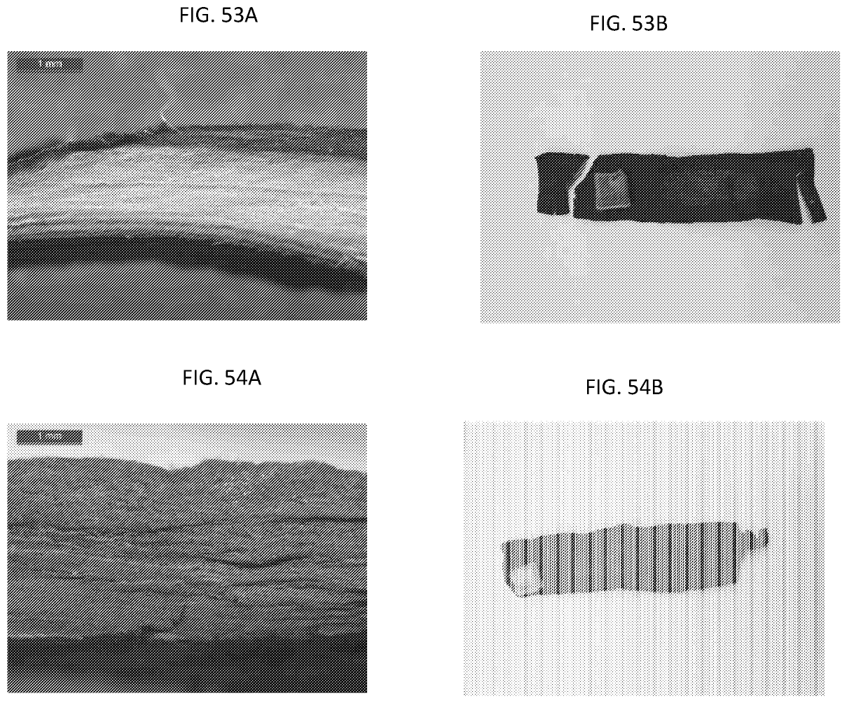

TABLE 10
Table 10. Protein Fixation and Dyeing Trials

| Trial | Conditions | Observations |
|---|---|---|
| 1 | 11 g/L Pea Protein | Appearance: |
| | Fixation pH = 4.0 | Good dye levelness and penetration |
| | FIG. 35A and 35B | Improved rub fastness results |
| | | Rub Fastness: |
| | | Staining: GSR 2 |
| | | Change to sample: GSR 3* |
| | | *small spot damage |
| 2 | 11 g/L Pea Protein | Appearance: |
| | Fixation pH = 5.0 | Good dye levelness |
| | FIG. 36A and 36B | Fairly good dye penetration with some |
| | | dye not being taken up on the |
| | | surface |
| | | Rub Fastness: |
| | | Staining: GSR 1-2 |
| | | Change to sample: GSR 2-3 |
| 3 | 11 g/L Pea Protein | Appearance: |
| | Fixation pH = 6.0 | Good dye levelness |
| | FIG. 37A and 37B | Fairly good dye penetration with some |
| | | dye not being taken up on the |
| | | surface |
| | | Rub Fastness: |
| | | Staining: GSR 2 |
| | | Change to sample: GSR 3-4 |
| 4 | 110 g/L Pea Protein | Appearance: |
| | Fixation pH = 4.0 | Viscosity of the protein solution |
| | FIG. 38A and 38B | considered to be too thick at 110 g/L |
| | | resulting in no/limited uptake of |
| | | protein into the mycelial material |
| | | Poor dye levelness, patchy appearance |
| | | due to residual protein on surface |
| | | creating a barrier on the material |
| | | Inconsistent dye penetration |
| | | Rub Fastness: |
| | | Staining: GSR 2 |
| | | Change to sample: GSR 2-3 |
| 5 | 11 g/L Soya Protein | Appearance: |
| | 5 g/L Dye (pH 10.0) + | Even dye coverage on one surface of |
| | Vegetable Glycerine | sample - reverse side irregular |
| | Fix at pH 4.0 | coverage |
| | (formic acid) | Dye only penetrated through one side |
| | FIG. 39A and 39B | of sample - likely related to |
| | | irregularities in morphology |
| | | Delamination of material occurring |
| | | Sample exhibited weakness during wet |
| | | processing causing the sample |
| | | to break, see supporting image |
| | | Feel: |
| | | Limited flexibility correlating |
| | | to inconsistent density |
| | | Rub fastness: |
| | | Staining: GSR 1-2 |
| | | Change to sample: GSR 1 |
| | | Significant flaking on area where rubbed |
| 6 | 11 g/L Soya Protein | Appearance: |
| | 5 g/L Dye (pH 10.0) + | Even dye coverage over the whole sample |
| | Fatliquor (1:3 AMC:DXV) | Inconsistent dye penetration however, |
| | Fix at pH 4.0 | dye observed in centre of the |
| | (formic acid) | sample. |
| | FIG. 40A and 40B | Delamination of material occurring |
| | | Feel: |
| | | Soft and flexible with even density |
| | | Rub fastness: |
| | | Staining: GSR 2 |
| | | Change to sample: GSR 2 |
| 7 | 11 g/L Soya Protein | Appearance: |
| | 5 g/L Dye (pH 10.0) + | Even dye coverage over the whole sample |
| | Coconut Oil | Limited dye penetration |
| | Fix at pH 4.0 | Delamination of material occurring |
| | (formic acid) | Feel: |
| | FIG. 41A and 41B | Soft and flexible but with |
| | | inconsistent density |
| | | Some dry and crispy areas |
| | | Rub fastness: |
| | | Staining: GSR 1-2 |
| | | Change to sample: GSR 1-2 |
| 8 | 11 g/L Pea protein | Appearance: |
| | 5 g/L Dye (pH 10.0) + | Uneven dye coverage |
| | Vegetable Glycerine | Dye only penetrated through one side |
| | Fix at pH 4.0 | of sample - likely related to |
| | (formic acid) | irregularities in morphology |
| | FIG. 42A and 42B | Delamination of material occurring |
| | | Some areas exhibit patchiness - likely |
| | | caused by residual protein |
| | | Feel: |
| | | Limited flexibility - correlating |
| | | to inconsistent density |
| | | Some dry and crispy areas |
| | | Rub fastness: |
| | | Staining: GSR 2 |
| | | Change to sample: GSR 1 |
| | | Significant flaking on area where rubbed |
| 9 | 11 g/L Pea protein | Appearance |
| | 5 g/L Dye (pH 10.0) + | Even dye coverage over the whole sample |
| | Fatliquor (1:3 AMC:DXV) | Little dye penetration only visible |
| | Fix at pH 4.0 | one side of the sample |
| | (formic acid) | Cracking on the surface |
| | FIG. 43A and 43B | Feel: |
| | | Soft and flexible but with |
| | | inconsistent density |
| | | Rub fastness |
| | | Staining: GSR 2 |
| | | Change to sample: GSR 2 |
| 10 | 11 g/L Pea protein | Appearance: |
| | 5 g/L Dye (pH 10.0) + | Uneven dye coverage |
| | Coconut Oil | Limited dye penetration - only slightly |
| | Fix at pH 4.0 | visible one side of the |
| | (formic acid) | sample |
| | FIG. 44A and 44B | Cracking on the surface |
| | | Feel: |
| | | Limited flexibility correlating |
| | | to inconsistent density |
| | | Rub fastness: |
| | | Staining: GSR 1-2 |
| | | Change to sample: GSR 1-2 |
| | | Flaking on area where rubbed |
| 11 | 22 g/L Soya Protein | Appearance: |
| | 5 g/L Dye (pH 10.0) + | Uneven dye coverage |
| | Vegetable Glycerine | Dye only penetrated through one side |
| | Fix at pH 4.0 | of sample - likely related to |
| | (formic acid) | irregularities in morphology |
| | FIG. 45A and 45B | Cracking on the surface |
| | | Feel: |
| | | Major inconsistency in sample density |
| | | Rub fastness: |
| | | Staining: GSR 1-2 |
| | | Change to sample: GSR 1-2 |
| | | Significant flaking on area where rubbed |
| 12 | 22 g/L Soya Protein | Appearance: |
| | 5 g/L Dye (pH 10.0) + | Uneven dye coverage |
| | Fatliquor (1:3 AMC:DXV) | Dye only penetrated through one side |
| | Fix at pH 4.0 | of sample - likely related to |
| | (formic acid) | irregularities in morphology |
| | FIG. 46A and 46B | No issues with delamination or cracking |
| | | Feel: |
| | | Limited flexibility correlating |
| | | to inconsistent density |
| | | Rub fastness: |
| | | Staining: GSR 2 |
| | | Change to sample: GSR 2 |
| | | Some flaking where rubbed |
| 13 | 22 g/L Soya Protein | Appearance: |
| | 5 g/L Dye (pH 10.0) + | Uneven dye coverage |
| | coconut oil | Very little dye penetration |
| | Fix at pH 4.0 | Delamination of material occurring |
| | (formic acid) | Cracking on the surface |
| | FIG. 47A and 47B | Feel: |
| | | Limited flexibility correlating |
| | | to inconsistent density |
| | | Rub Fastness: |
| | | Staining: GSR 2 |
| | | Change to sample: GSR 2-3 |
| 14 | 22 g/L Pea protein | Appearance: |
| | 5 g/L Dye (pH 10.0) + | Uneven dye coverage |
| | Vegetable Glycerine | Dye only penetrated through one side of |
| | Fix at pH 4.0 | the material - related to |
| | (formic acid) | irregularities in morphology |
| | FIG. 48A and 48B | Delamination of material occurring |
| | | Cracking on the surface |
| | | Feel: |
| | | Major inconsistency in density |
| | | Rigid |
| | | Rub fastness: |
| | | Staining: GSR 1-2 |
| | | Change to sample: GSR 1-2 |
| | | Significant flaking on area where rubbed |
| 15 | 22 g/L Pea protein | Appearance: |
| | 5 g/L Dye (pH 10.0) + | Uneven dye coverage |
| | Fatliquor (1:3 AMC:DXV) | Dye only penetrated slightly through |
| | Fix at pH 4.0 | one side of sample - related to |
| | (formic acid) | irregularities in morphology |
| | FIG. 49A and 49B | Cracking on the surface |
| | | Feel: |
| | | Limited flexibility correlating to |
| | | inconsistent density |
| | | Rub fastness: |
| | | Staining: GSR 2 |
| | | Change to sample: GSR 1-2 |
| | | Significant flaking on area where rubbed |
| 16 | 22 g/L Pea protein | Appearance: |
| | 5 g/L Dye (pH 10.0) + | Even dye coverage on one side |
| | Coconut Oil | Small amount of dye penetrated through |
| | Fix at pH 4.0 | one side of the sample |
| | (formic acid) | Delamination of material occurring |
| | FIG. 50A and 50B | Cracking on the surface |
| | | Feel: |
| | | Limited flexibility correlating to |
| | | inconsistent density (less |
| | | flexible than sample 11) |
| | | Rub fastness: |
| | | Staining: GSR 1-2 |
| | | Change to sample: GSR 1-2 |
| | | Significant flaking on area where rubbed |
| 17 | 22 g/L Pea protein | Appearance: |
| | 5 g/L Dye (pH 10.0) | Even dye coverage on one side |
| | Fix at pH 4.0 | Small amount of dye penetrated through |
| | (formic acid) | one side of the sample - |
| | FIG. 51A and 51B | related to irregularities in morphology |
| | | Delamination of material occurring |
| | | Cracking on the surface |
| | | Feel: |
| | | Sample hard all over |
| | | Rub fastness: |
| | | Staining: GSR 1 |
| | | Change to sample: GSR 1-2 |
| 18 | 11 g/L Soya Protein | Appearance: |
| | 5 g/L Dye (pH 10.0) | Uneven dye coverage |
| | Fix at pH 4.0 | Dye only penetrated through one side |
| | (formic acid) | Cracking on the surface |
| | FIG. 52A and 52B | Feel: |
| | | Sample hard all over |
| | | Rub fastness: |
| | | Staining: GSR 1-2 |
| | | Change to sample: GSR 1-2 |
| 19 | 22 g/L Soya Protein | Appearance: |
| | 5 g/L Dye (pH 10.0) | Even dye coverage on one side |
| | Fix at pH 4.0 | No dye penetration |
| | (formic acid) | Cracking on the surface |
| | FIG. 53A and 53B | Feel: |
| | | Sample hard all over |
| | | Rub fastness: |
| | | Staining: GSR 1 |
| | | Change to sample: GSR 2 |
| 20 | 11 g/L Pea Protein | Appearance |
| | 5 g/L Dye (pH 10.0) | Even dye coverage |
| | Fix at pH 4.0 | Dye penetrated well throughout material |
| | (formic acid) | Lighter finish |
| | This sample was left | Feel |
| | dying overnight | Rigid, not flexible and hard |
| | which has given better | Would snap upon flexing |
| | results in the | Rub fastness |
| | dye penetration. | Staining: GSR 2 |
| | FIG. 54A and 54B | Change to sample: GSR 3-4 |

Samples 13-16 had increased protein. All three samples performed poorly on the rub fastness test and had limited dye penetration. Without wishing to be bound by theory, this may be due to the extra protein sitting on the surface, creating a barrier and preventing the dye from being able to migrate into the materials structure. Samples 17-20 contained no plasticizing agents and represent control sample of the samples 1-16. All of the control samples (17-20) exhibited hardness and poor flexibility. Samples 17-19 had poor rub fastness results. However, sample 20 had improved rub fastness results and dye penetration. This difference is likely due to the fact that sample 20 was produced during the first set of trials in which performance observations differ from the later samples due to the length of time the samples were left in the dye solution.

Samples were also dyed and then washed or not washed. Results from these trials are included as Table 11.

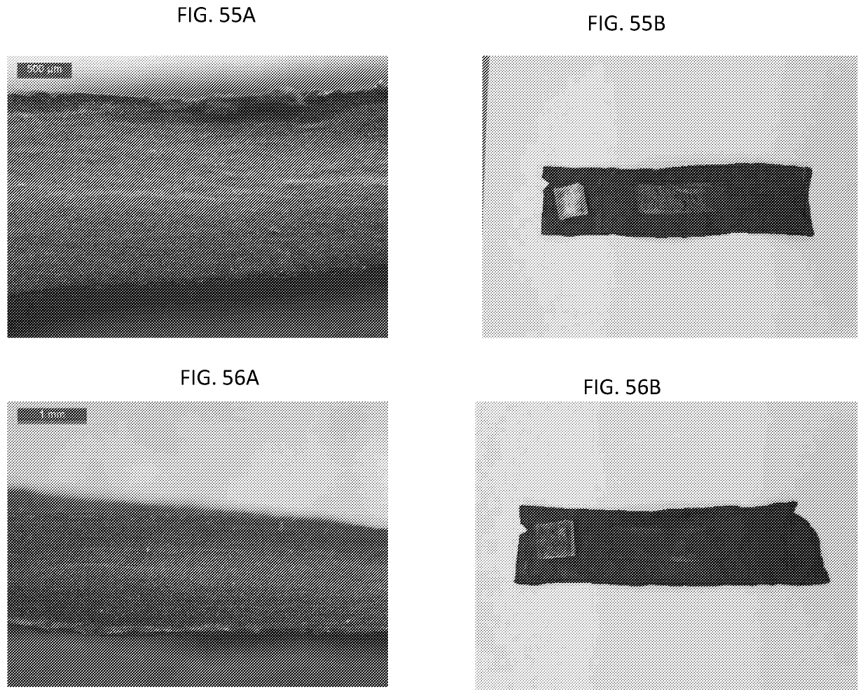

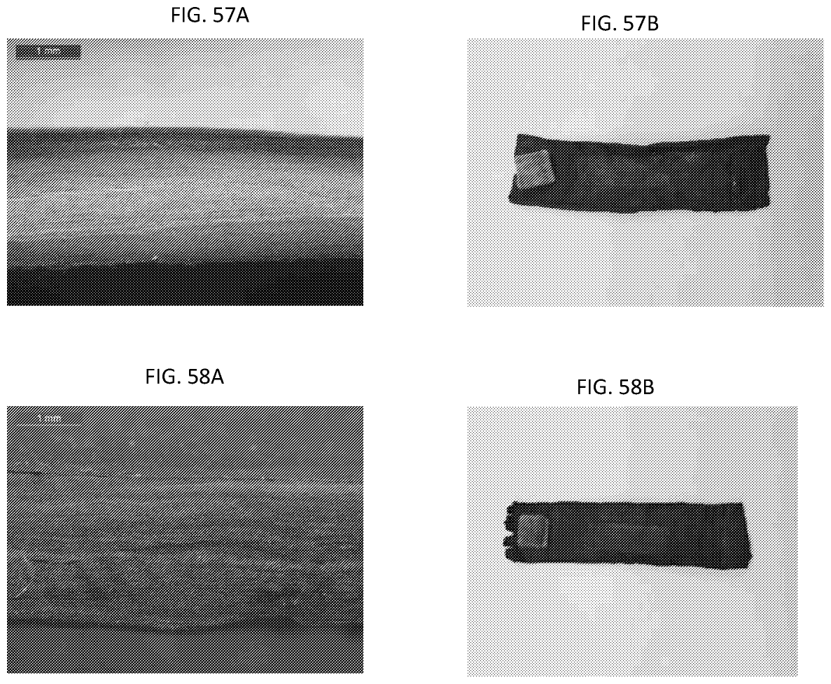

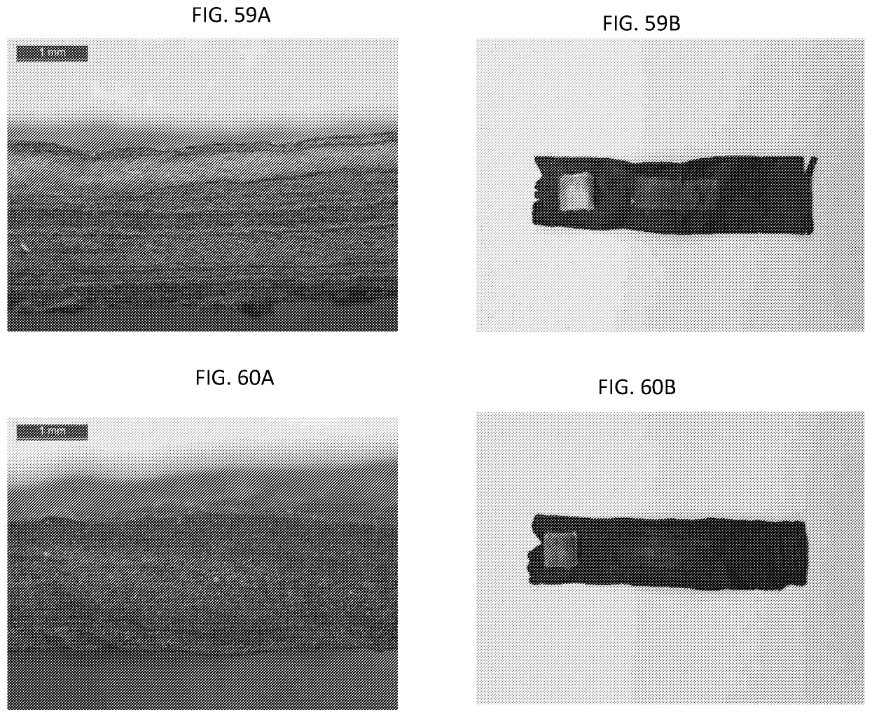

TABLE 11

| Trial | Conditions | Observations |
|---|---|---|
| 21 | 2a Washed (W) 11 g/L Soya | Appearance: |
| | protein 5 g/L Acid Brown Dye | Uniform dye coverage across the whole sample |
| | 11 g/L Fatliquor | Good dye coverage with slight variation |
| | (1:3 AMC:DXV) | across the sample |
| | FIG. 55A and 55B | Small amount of cracking on the surface |
| | | Feel: |
| | | Soft, flexible and even density |
| | | Rub Fastness: |
| | | Staining: GSR 1-2 |
| | | Change to sample: GSR 1-2 |
| | | Significant flaking where rubbed |
| 22 | 2b Not Washed (NW) 11 g/L Soya | Appearance: |
| | protein 5 g/L Acid Brown Dye | Uniform dye coverage across the whole sample |
| | 11 g/L Fatliquor | Very good dye penetration |
| | (1:3 AMC:DXV) | Small amount of cracking on the surface |
| | FIG. 56A and 56B | Feel: |
| | | Soft, flexible and even density |
| | | Rub Fastness: |
| | | Staining: GSR 1 |
| | | Change to sample: GSR 2 |
| 23 | 5a W 11 g/L Pea protein | Appearance: |
| | 5 g/L Acid Brown Dye | Uniform dye coverage on one side |
| | 11 g/L Fatliquor | Limited dye penetration |
| | (1:3 AMC:DXV) | Hard in some areas but no cracks |
| | FIG. 57A and 57B | observed (cracks appear on flexing) |
| | | Feel: |
| | | Uneven softness, not flexible |
| | | and uneven density |
| | | Rub Fastness: |
| | | Staining: GSR 1-2 |
| | | Change to sample: GSR 2 |
| 24 | 5b NW 11 g/L Pea protein | Appearance: |
| | 5 g/L Acid Brown Dye | Uniform dye coverage across |
| | 11 g/L Fatliquor | the whole sample |
| | (1:3 AMC:DXV) | Very good dye penetration |
| | FIG. 58A and 58B | No cracking or delamination |
| | | Feel: |
| | | Soft, flexible and even density |
| | | Rub Fastness: |
| | | Staining: GSR 1 |
| | | Change to sample: GSR 2-3 |
| 25 | Control, No protein W (CW) | Appearance: |
| | 0 g/L protein | Uniform dye coverage on one side |
| | 5 g/L Acid Brown Dye | Good dye penetration with a lighter tone |
| | 11 g/L Fatliquor | Thin sample with cracks and a |
| | (1:3 AMC:DXV) | small hole formed in the middle |
| | FIG. 59A and 59B | Feel: |
| | | Soft, flexible and uneven density |
| | | Rub Fastness: |
| | | Staining: GSR 2 |
| | | Change to sample: GSR 1-2 |
| | | Significant flaking where rubbed |
| 26 | Control, No protein NW (CNW) | Appearance: |
| | 0 g/L protein | Uniform dye coverage across |
| | 5 g/L Acid Brown Dye | the whole sample |
| | 11 g/L Fatliquor | Very good dye penetration |
| | (1:3 AMC:DXV) | No cracking or delamination |
| | FIG. 60A and 60B | Feel: |
| | | Soft, flexible and even density |
| | | Rub Fastness: |
| | | Staining: GSR 1 |
| | | Change to sample: GSR 2 |

Further trials were conducted with an increased sample size. Batch 2044 was used in trial 27. The result is shown in Table 12.

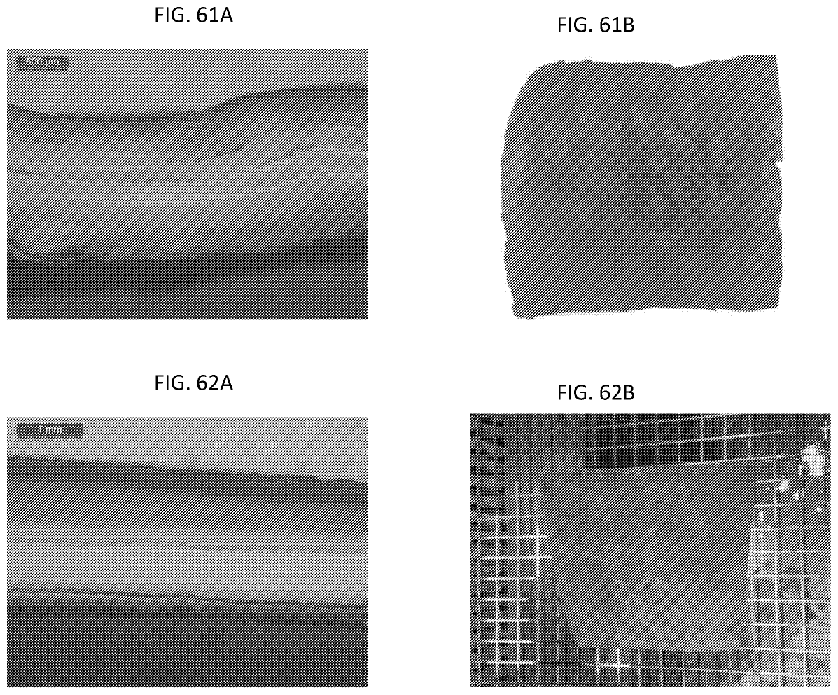

TABLE 12

| Trial | Conditions | Observations |
|---|---|---|
| 27 | 11 g/L Soya protein | Appearance |
| | 5 g/L Acid Brown Dye | Even dye across whole sample. |
| | 11 g/L Fatliquor | No dye penetration |
| | (1:3 AMC:DXV) | Feel |
| | FIG. 61A and 61B | Soft and flexible however, has some |
| | | areas have a hard, inflexible feel |
| | | to them |
| 28 | 4 NW Large Samples | Appearance |
| | 2.5 g/L Dye | Uneven/patchy dye coverage possibly due |
| | 11 g/L Soya | to residual soya protein |
| | 11 g/L Fatliquor | on the accumulating on the surface |
| | (1:3 AMC:DXV) | Variable penetration due to inconsistent |
| | 200 mg/L Fungicide Dried | density and new mechanical |
| | (Ambient) 50 g/L Fatliquor | technique |
| | FIG. 62A and 62B | Feel |
| | | Flexible |
| | | Soft, but could be enhanced |

Trials were conducted with lower dye concentrations, to assess the possibility of removing the washing step. Batch 2373 was used in these trials. The results are shown in Table 13.

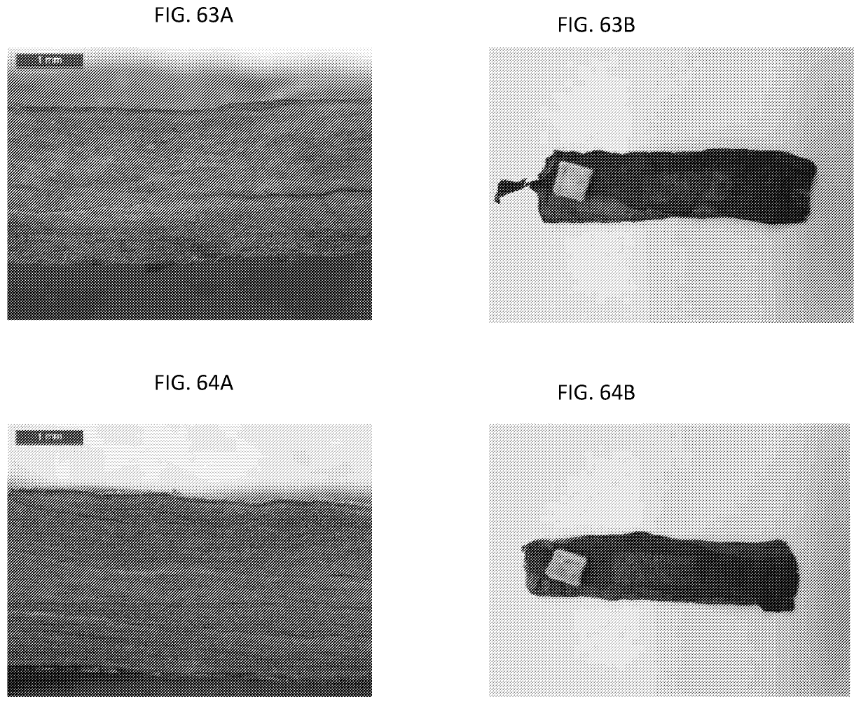

TABLE 13

| Trial | Conditions | Observations |
|---|---|---|
| 29 | 1 NW | Appearance |
| | 2.5 g/L Dye | Uneven dye coverage/patchy |
| | 11 g/L Soya | appearance |
| | 11 g/L Fatliquor | Cracking on surface |
| | (1:3 AMC:DXV) | Even dye penetration, with |
| | 200 mg/L Fungicide | a lighter tone |
| | FIG. 63A and 63B | Feel |
| | | Hard but flexible in comparison |
| | | to washed sample |
| | | Rub Fastness |
| | | Staining: GSR 2 |
| | | Change to sample: GSR 2-3 |
| 30 | 2 W | Appearance |
| | 5.0 g/L Dye | Uneven dye coverage/patchy appearance |
| | 11 g/L Soya | Cracking on surface |
| | 11 g/L Fatliquor | Very good dye penetration |
| | (1:3 AMC:DXV) | Feel |
| | 200 mg/L Fungicide | Hard and not flexible |
| | FIG. 64A and 64B | Rigid |
| | | Rub fastness |
| | | Staining: GSR 2 |
| | | Change to sample: GSR 3-4 |
| | | Flaking where rubbed |

Next, a vegetable tannin (mimosa) was used to dye the cultivated mycelium material. Batch 2342 was used in these trials. The results are shown in Table 14.

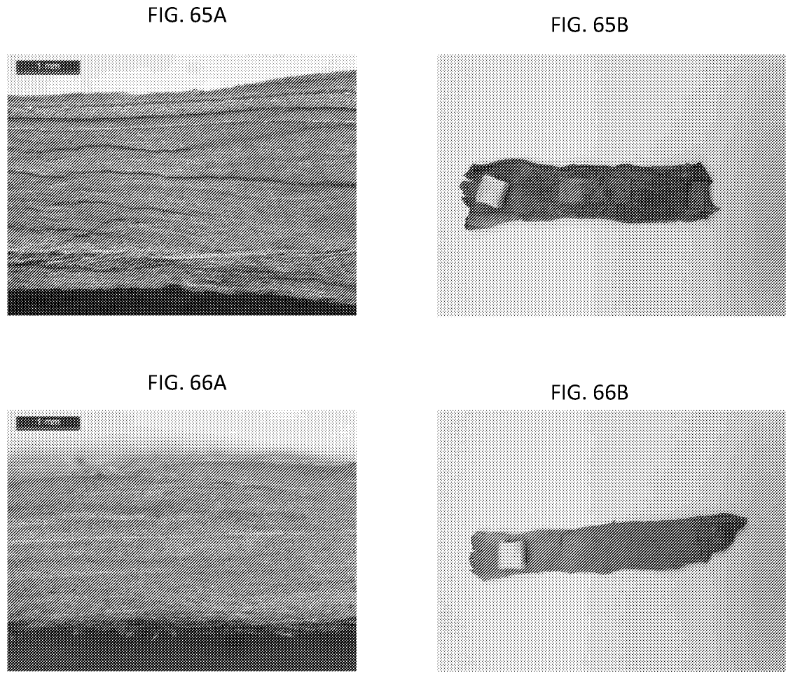

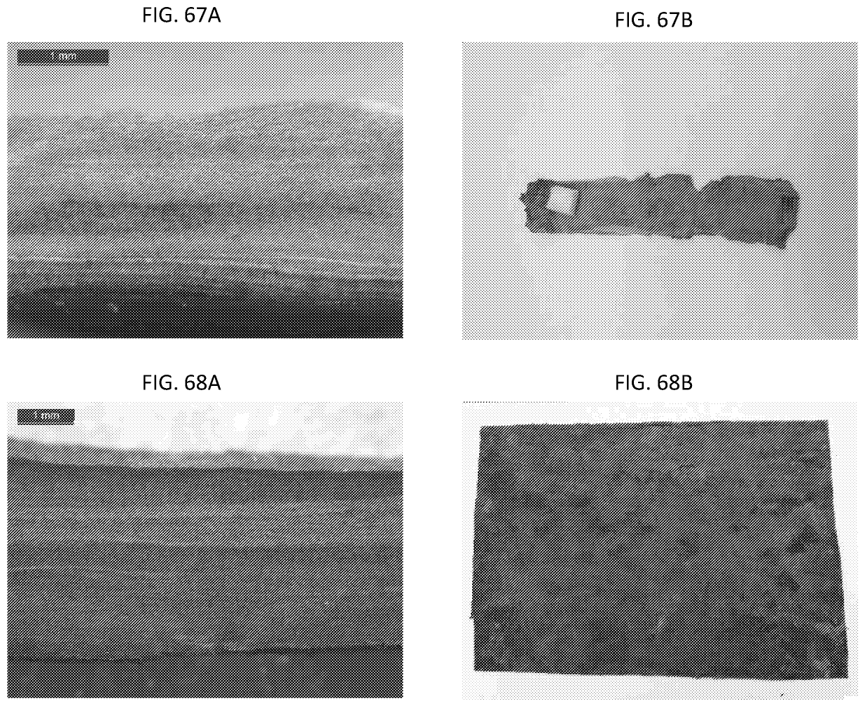

TABLE 14

| Trial | Conditions | Observations |
|---|---|---|
| 31 | Veg - a NW | Appearance |
| | 500 g Veg Tan (mimosa) | Uneven/patchy dye coverage |
| | in 3 L water | Cracking on the surface |
| | 2.5 g/L Dye | Darker tone of brown in comparison to |
| | 11 g/L Soya protein | other veg tanned samples |
| | 11 g/L Fatliquor | Good penetration of dye |
| | (1:3 AMC:DXV) | Feel |
| | 21 g/3 L MgSO₄ | Hard and not flexible |
| | 200 mg/L Fungicide | Rigid |
| | Control | Rub fastness |
| | FIG. 65A and 65B | Staining: GSR 2-3 |
| | | Change to sample: GSR 2-3 |
| | | Some flaking where rubbed |
| 32 | Veg - b NW | Appearance |
| | 500 g Veg Tan (mimosa) | Even dye coverage |
| | in 3 L water | Pale shade of brown |
| | 2.5 g/L Dye | Good penetration of dye |
| | 11 g/L Soya protein | Feel |
| | 11 g/L Fatliquor | Soft in comparison to Veg - a |
| | (1:3 AMC:DXV) | Flexible |
| | 21 g/3 L MgSO₄ | Rub fastness |
| | 200 mg/L Fungicide | Staining: GSR 4 |
| | Glutaraldehyde | Change to sample: GSR 4 |
| | FIG. 66A and 66B | |
| 33 | Veg - c NW | Appearance |
| | 500 g Veg Tan (mimosa) | Uneven/patchy dye coverage |
| | in 3 L water | Okay penetration, needs optimisation |
| | 2.5 g/L Dye | Pale with dark patches |
| | 11 g/L Soya protein | Feel |
| | 11 g/L Fatliquor | Very soft |
| | (1:3 AMC:DXV) | Flexible |
| | 21 g/3 L MgSO₄ | Rub fastness |
| | 200 mg/L Fungicide | Staining: GSR 3-4 |
| | Over Fatliquor | Change to sample: GSR 4 |
| | (110 g/L) | |
| | FIG. 67A and 67B | |
| 34 | 50 g/L vegetable | Appearance |
| | tannin (mimosa) | Some patchiness on the surface. |
| | in 1.5 L water | Very good dye penetration |
| | 2.5 g/L Acid Brown Dye | Feel |
| | 10 g/L MgSO₄ | Flexible |
| | 100 g/L Fatliquor | Very soft on one side with skin |
| | (1:3 AMC:DXV) | like surface giving a slightly |
| | 150 mg/L Fungicide | harder texture. |
| | (Busan 30 WB) | |
| | FIG. 68A and 68B | |

The presence of vegetable tannins resulted in increased uptake of the dye and provided the material with a more robust structure. High concentrations of the vegetable tannins made the mycelium material feel firmer and reduced flexibility. This is similar to vegetable tanned leathers which are typically used for firm sole leathers, belts bridle leathers etc. Therefore the concentration of vegetable tannin used should be reduced to increase the flexibility of the dyed mycelium material.

An exemplary dye and vegetable tanning procedure as performed is shown below in Table 15.

TABLE 15
Dye & Vegetable Tan
1. Mix the vegetable tannin/dye solution

| Quantity | Chemical | Supplier |
|---|---|---|
| 75 g (50 g/L) | Mimosa (vegetable tannin) | Forestal Mimosa |
| 3.75 g (2.5 g/L) | Acid Brown 425 | BASF - Stahl |
| 1.5 L | Warm Water (40° C.) | Mains Water |
| Approx. 65-75 g | Mycelial Material | Bolt Threads - Ecovative |

2. Pour Solution over a half panel of mycelium material.
3. Gently massage (by hand) the mycelial material (using squeezing action) on one side of the material
to aid in the uptake of the dye tannin/solution. After 15 minutes, repeat the
massaging action on the reverse side of the sample.
4. Leave the mycelial material in the solution until the solution has penetrated through the full thickness
of the material - approximately 3 hours.
Stages 3 and 4 can be accelerated with the use of rollers on an industrial scale

Tannin Precipitation

| Quantity | Chemical | Supplier |
|---|---|---|
| 15 g (10 g/L) | Magnesium Sulfate (Mg₂SO₄) | Sigma Aldrich |

5. Add the magnesium sulfate to the dye/tannin solution and massage for 15 minutes.
6. Leave the sample to soak in solution for an additional 30 minutes.
Dye Fixation
7. After soaking the dye and vegetable tanning is fixed using formic acid - pH is adjusted pH
4.0 with gradual additions and monitored using a pH electrode.
8. Samples were left in solution and allowed to fix for 1 hour.
Fatliquoring
9. A fatliquor blend and fungicide was added to warm water:

| Quantity | Chemical | Supplier |
|---|---|---|
| 50 g (33.3 g/L) | Trupon AMC (Fatliquor) | Trumpler |
| 100 g (66.6 g/L) | Trupon DXV (Fatliquor) | Trumpler |
| 225 mg (150 mg/L) | Busan 30 WB (Fungicide) | Buckman |
| 1.5 L | Warm Water (50° C.) | Mains Water |

10. The mycelial sample was removed from the dye solution, lightly rinsed with water (no
squeezing action - briefly under a tap) and transferred to the fatliquor/fungicide solution.
11. The mycelial material was gently massage by hand (using squeezing action) on one side of the
material to aid in the uptake of the dye tannin/solution. After 15 minutes, the massaging action was
repeated on the reverse side of the sample.
12. Samples were left to soak in the fatliquor/fungicide solution until the emulsion broke (this
can take up to 3.5 hours).
13. In cases where the fatliquor solution does not break salt (sodium chloride - NaCl) can be
added to aid with the breaking of the emulsion (10 g/L).
14. After fatliquoring, samples were lightly rinsed (no squeezing action - briefly under a tap)
Drying
15. Samples were rinsed and air dried at ambient conditions until dry. Due to the nature of
swelling the mycelial samples can take up to 2-days to dry.
Compression
16. Once dry samples underwent compression using a hydraulic press (50° C. at 50 kg/cm²) to both
sides of the mycelial material.

After drying, the mycelium material was rinsed with water again then dried using paper towels. The materials were then pressed at 50° C. at 100 kg/cm². This resulted in a less intense color but a much-improved finish

Further trials with additional color dyes, Luganil Bordeaux B, Luganil Red EB, Luganil Yellow G, and Luganil Olive Brown N, were performed. The results are shown in Table 16.

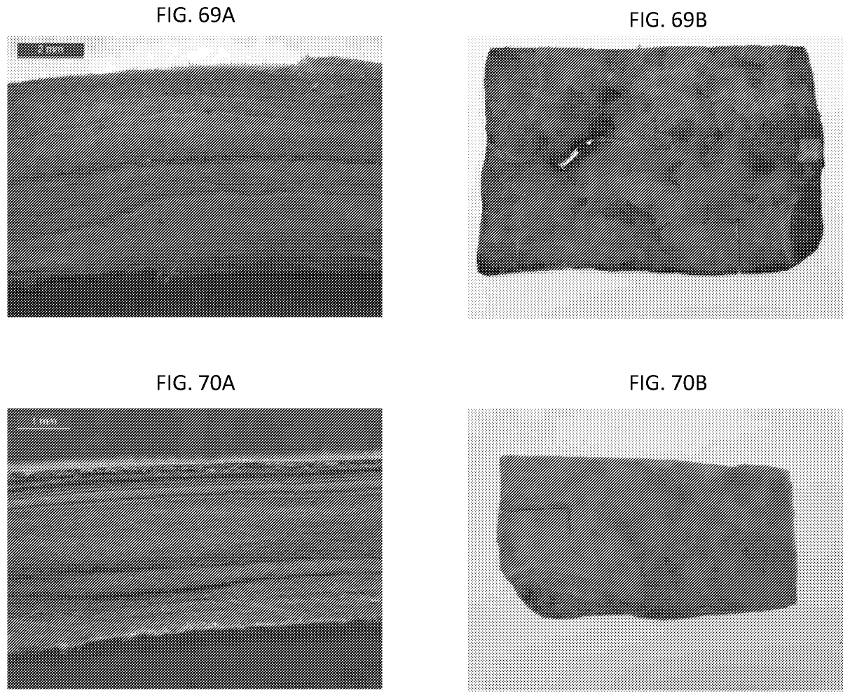

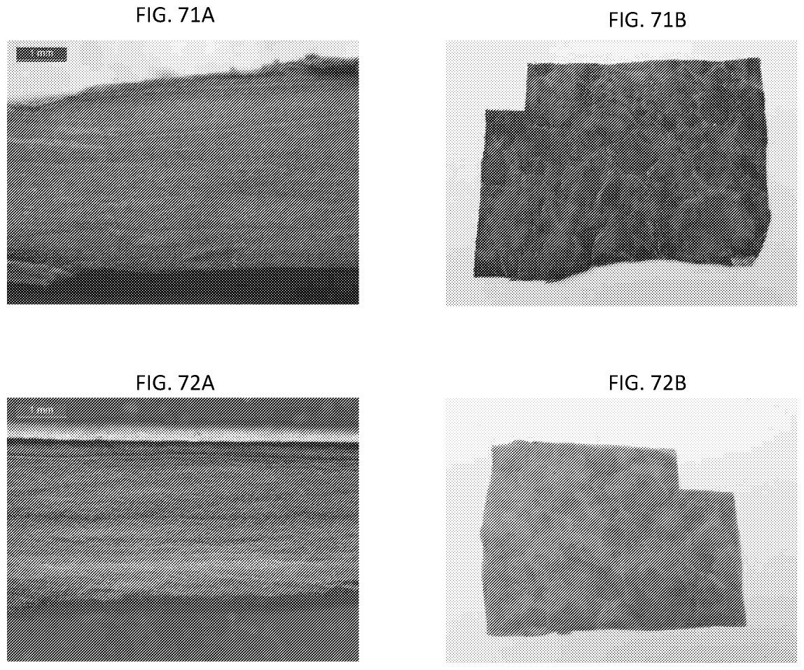

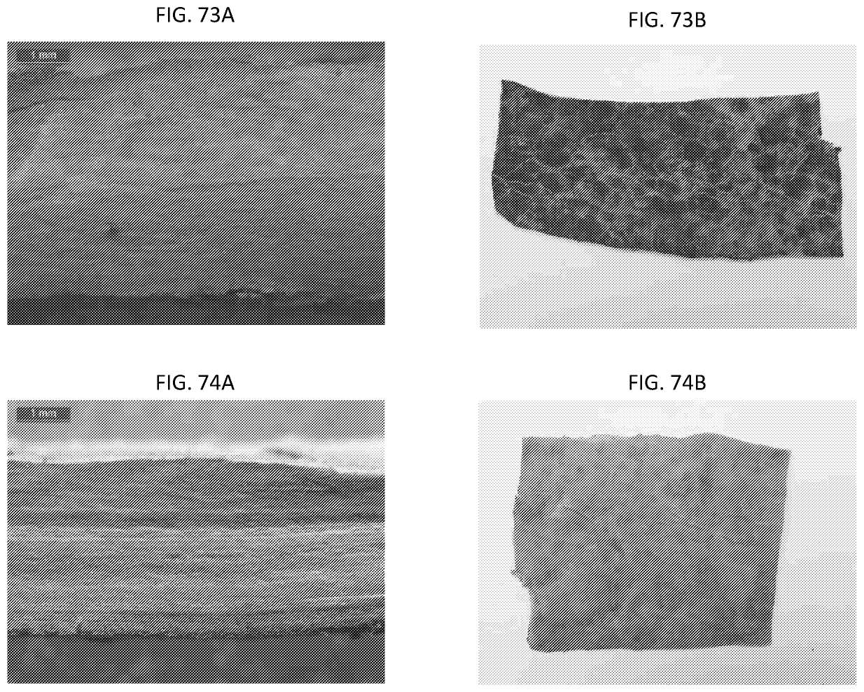

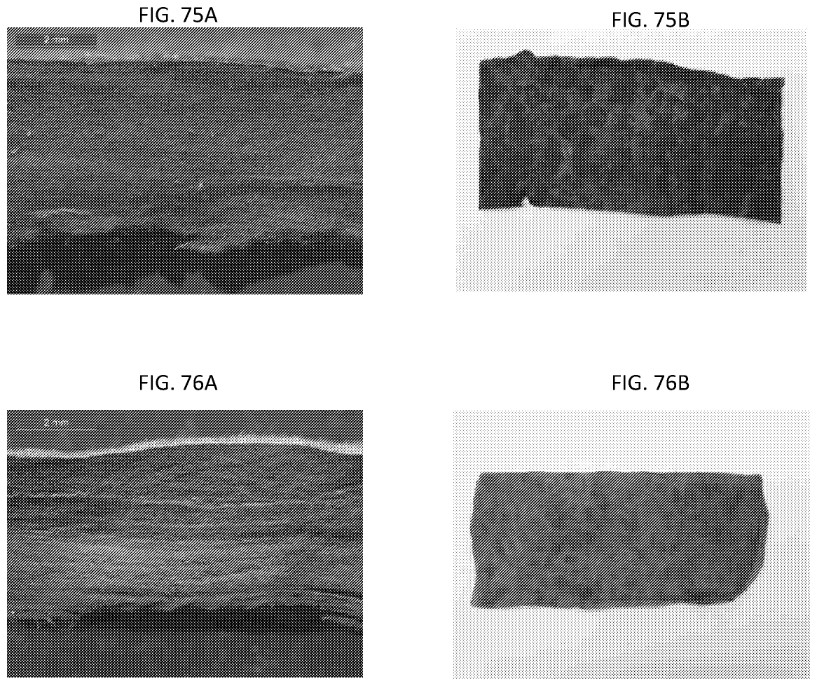

TABLE 16

| Trial | Conditions | Observations |
|---|---|---|
| 35 | Luganil Bordeaux B | Appearance |
| | Before Wash | Uneven dye coverage with |
| | FIG. 69A and 69B | some very dark patches |
| | | Very good dye penetration |
| | | Feel |
| | | Firm but flexible |
| | | Soft on one surface with the |
| | | underside giving a rough texture |
| 36 | Luganil Bordeaux B | Appearance |
| | Additional Wash | Improved even dye coverage on the surface. |
| | (after drying) | However, uneven coloring |
| | Compression at 100 kg/cm² | on the underside of the material |
| | FIG. 70A and 70B | Very good dye penetration |
| | | Feel |
| | | Firm but flexible |
| | | Soft on both sides with slight roughness |
| | | on the underside |
| 37 | Luganil Red EB | Appearance |
| | Before Wash | Uneven dye coverage with many very |
| | FIG. 71A and 71B | dark patches |
| | | Very good dye penetration |
| | | Feel |
| | | Firm but flexible |
| | | Soft on one surface with the underside |
| | | giving a rough texture |
| 38 | Luganil Red EB | Appearance |
| | With wash and Pressed | Much more even dye coverage on the surface. |
| | at Additional Wash | However, uneven |
| | (after drying) | coloring on the underside of the material |
| | Compression at 100 kg/cm² | Very good dye penetration |
| | FIG. 72A and 72B | Feel |
| | | Flexible |
| | | Soft on both sides |
| 39 | Luganil Yellow G | Appearance |
| | Before Wash | Uneven dye coverage |
| | FIG. 73A and 73B | Cracking of dye on the surface |
| | | Many dark patches across the sample |
| | | Very good dye penetration |
| | | Feel |
| | | Flexible |
| | | Cracking of dye on surface creating a |
| | | rough texture |
| 40 | Luganil Yellow G | Appearance |
| | With wash and | Even dye on one side with skin like side |
| | Pressed | remaining patchy |
| | at Additional Wash | Good dye penetration with some darker shades |
| | (after drying) | Feel |
| | Compression at 100 kg/cm² | Soft on both sides with slight roughness |
| | FIG. 74A and 74B | still present on the skinlike |
| | | side. |
| | | Flexible |
| 41 | Luganil Olive Brown N | Appearance |
| | Before Wash | Patchiness on the surface with many areas |
| | FIG. 75A and 75B | with a darker appearance. |
| | | Very good dye penetration |
| | | Feel |
| | | Flexible but rough with very hard |
| | | bits around the edge |
| 42 | Luganil Olive Brown N | Appearance |
| | With wash and Pressed | Even dye coverage with some patchiness |
| | at Additional Wash | observed on the underside |
| | (after drying) | Good dye penetration with a slight |
| | Compression at 100 kg/cm² | lighter appearance in the middle |
| | FIG. 76A and 76B | Feel |
| | | Soft on both sides |
| | | Flexible |

### Example 5

### Treatment of Cultivated Mycelium Material with Dye Solution and Protein Solution

Mats of cultivated mycelium material preserved using Treatment A as described in Example 1 were treated with a number of different finishing agents. Various finishes were applied in varying orders and the aesthetic and functional appearance of the cultivated mycelium material was evaluated. The finish protocols are shown in Table 17. Various finishes including the following were applied:
A nitrocellulose (LW65-370-Stahl) and protein polishable finish was applied following the application of a vegetable glycerol plasticizer followed by rolling the cultivated mycelium material in perpendicular directions to create a “box grain” effect
A nitrocellulose (LW65-370-Stahl) was mixed with water in even ratios by weight with a handle modifier (HM-4896);
A conventional polyurethane finish comprising a base coat of pigment, wax emulsion, cationic wax, acrylic resin, aqueous urethane resin and water. The base coat was followed by a top coat comprising water lacquer, aqueous lacquer and handle modifier.
An antique effect finish comprising the conventional polyurethane finish as described above with an antique effect coat applied between the base coat and the top coat. Surface buffing was also applied to the cultivated mycelium material.
A distressed effect finish comprising the antique effect finish with additional buffing steps and additional topcoat
An embossed sample comprising a cultivated mycelium material dyed with Luganil olive brown dye that was embossed using a silicone mat at 50 degrees Celsius under 100 kg/cm³.
Various plant protein finishes alone or with a crosslinker
Carnauba wax alone or in combination with other known finishes.

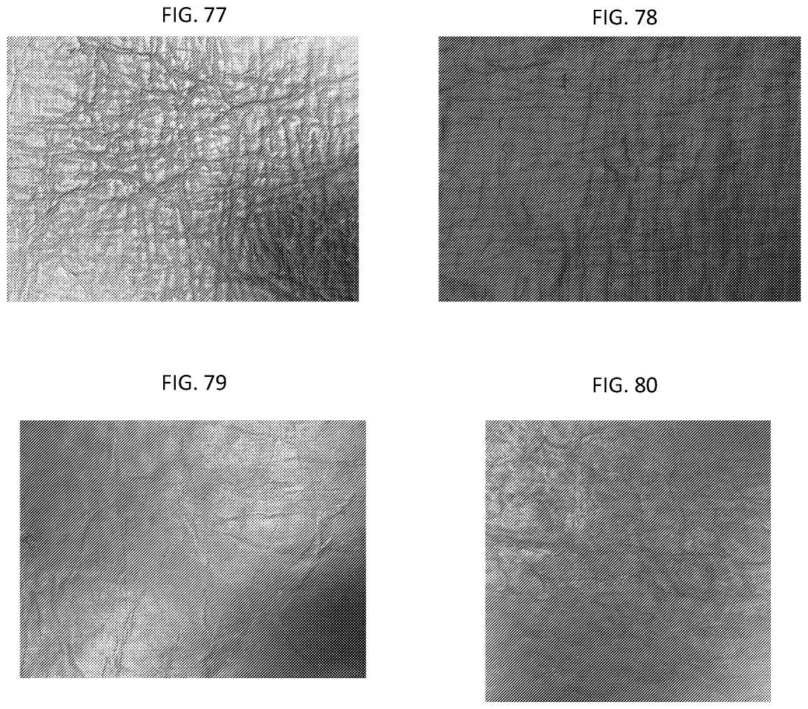

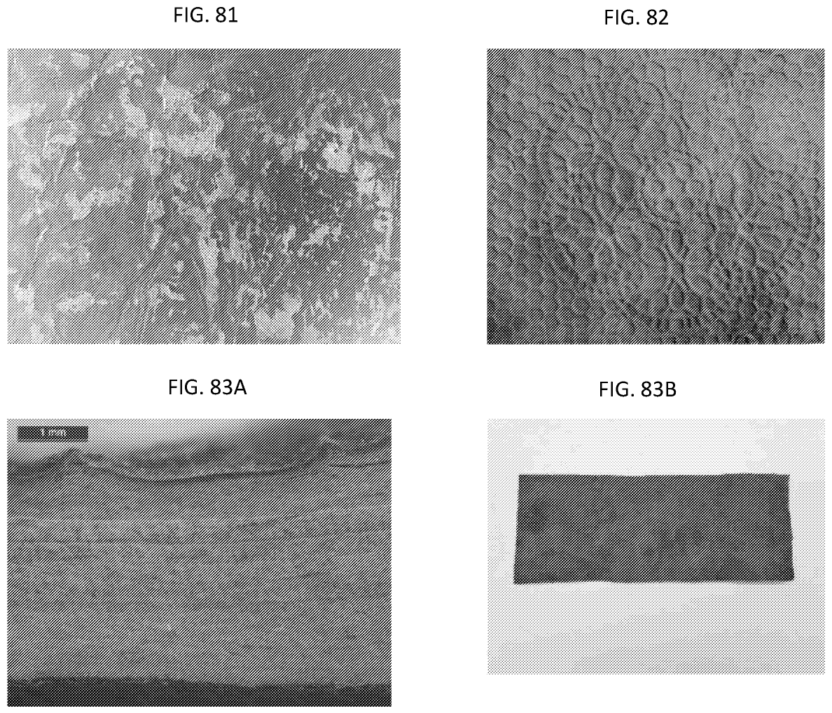

An example of Nitrocellulose and Protein Polishable Finish—Box Effect is shown in FIG. 77. An example of Nitrocellulose Finish—Box Effect is shown in FIG. 78. An example of Conventional polyurethane finish is shown in FIG. 79. An example of Antique Effect is shown in FIG. 80. An example of Distressed Effect is shown in FIG. 81. An example of Embossed Luganil Olive Brown is shown in FIG. 82.

TABLE 17
Table 17: Finishing Protocols

1. Nitrocellulose and Protein Polishable Finish - Box Effect
Vegetable glycerine plasticiser applied by hand to the surface
Nitrocellulose (LW65-370 - Stahl) and protein polishable finish applied
Box grain effect - the mycelial material ‘grain side in’ (finished side in) is rolled in one direction and
then the perpendicular to create a box effect
2. Nitrocellulose Finish - Box Effect

| Product | Property | Amount by Weight |
|---|---|---|
| LW65-370 (Stahl) | Clear natural warm handle | 50 |
| Nitrocellulose | | |
| Water | — | 50 |
| Handle Modifier | Various available depending on desired feel | 5 g/100 g Finish Mixture |
| | HM-4896 for a grabby feel during trials | |

Nitrocellulose finish applied.
Box grain effect - the mycelial material ‘grain side in’ (finished side in) is rolled in one direction and
then the perpendicular to create a box effect
3. Conventional Polyurethane Finish
Samples plated at 50° C. at 100 kg/cm2 (using a hydraulic press)
Base Coat x 2:

| Product | Property | Amount by Weight |
|---|---|---|
| Pigment | Color and coverage | 100 |
| Wax Emulsion | Good filling/soft waxy | 90 |
| Cationic Wax | Helps hold up/covering/warm soft natural feel | 70 |
| Acrylic Resin | Good flexibility/good cover without overfill | 230 |
| Aqueous Urethane Resin | Good extensibility/thin film/natural feel | 130 |
| Water | | 380 |

Plated at 50° C. at 50 kg/cm2
Additional base coat x 2
Top coat x 1

| Product | Property | Amount by weight |
|---|---|---|
| Water Lacquer | Light natural feel/soft waxy feel | 275 |
| Aqueous Lacquer | Good for ironing/good for rub fastness/smooth | 275 |
| | surface | |
| Handle Modifier | Grabby | 40 |
| Water | — | 410 |

Sample kiss plated using hydraulic press (quick release of press) to seal finish
4. Antique Effect
Samples plated at 50° C. at 100 kg/cm2 (using a hydraulic press)
Base Coat x 2

| Product | Property | Amount by weight |
|---|---|---|
| Pigment | Colour and coverage | 100 |
| Wax Emulsion | Good filling/soft waxy | 90 |
| Cationic Wax | Helps hold up/covering/warm soft natural feel | 70 |
| Acrylic Resin | Good flexibility/good cover without overfill | 230 |
| Aqueous Urethane Resin | Good extensibility/thin film/natural feel | 130 |
| Water | — | 380 |

Plated at 50° C. at 50 kg/cm2
Additional base coat x 2
Antique Effect Coat x 1 - Black pigment and water only
Top coat x 1:

| Product | Property | Amount by weight |
|---|---|---|
| Water Lacquer | Light natural fccl/soft waxy feel | 275 |
| Aqueous Lacquer | Good for ironing/good for rub fastness/smooth | 275 |
| | surface | |
| Product | Property | Amount by weight |
| Waterborne polyurethane dispersion | High gloss effect, gives excellent flexibility | 200 |
| (WT-2511 Stahl) | and rub fastness | |
| Waterborne polyurethane dispersion | Glossy with a waxy soft feel and a ‘non- | 50 |
| (WT-2524 Stahl) | squeaking’ finish | |
| Hand modifier | Increases resistance to wet and dry rubs | 20 |
| (HM-13-363) | | |
| Water | | 500 |
| Handle Modifier | Grabby | 40 |
| Water | — | 410 |

5. Distressed Effect
‘Antique Effect’ plus surface buffing and additional topcoat applied
6. Embossed Luginal Olive Brown
Sample produced after wash stage in trial 10.4
Embossed using mat at 50° C. under 100 kg/cm2 - pressure whilst sample is still slightly wet

The protocol for the oily pull up top coat is shown in Table 18.

Results from these trials are shown in Table 19. Finishes were mainly evaluated based on aesthetic appearance and hand feel. Many finishes produced a desirable appearance and hand feel. Microscopy images of a cross section of each material are shown in panel A of each figure and a macroscopic view of the material is shown in panel B of each figure.

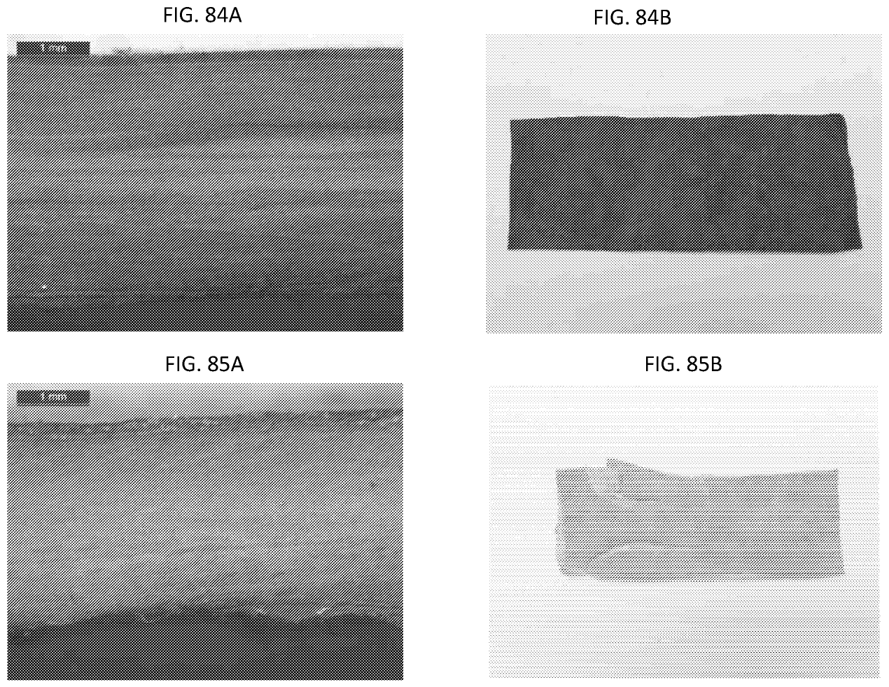

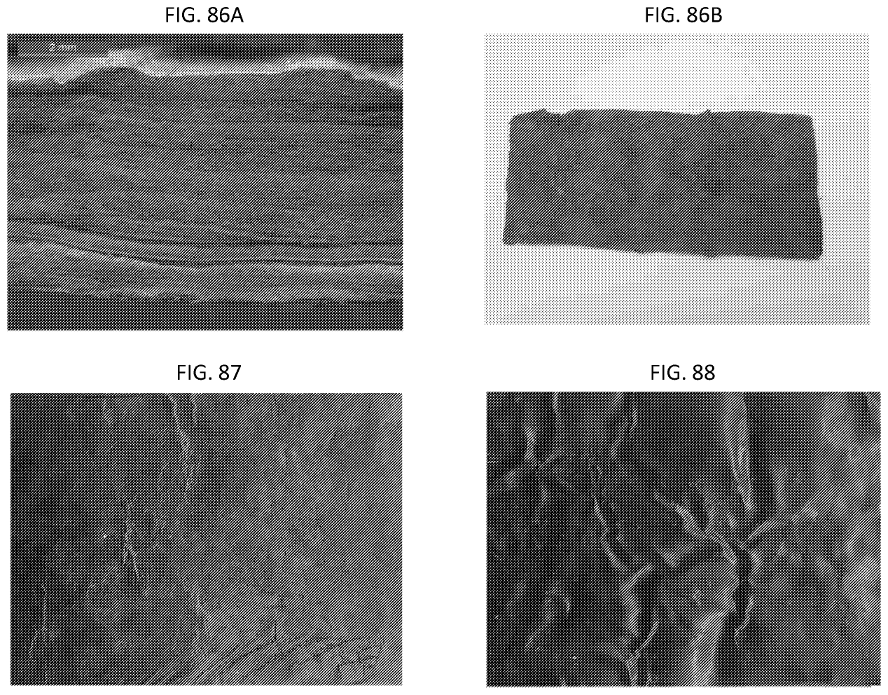

TABLE 19

| Trial | Conditions | Observations |
|---|---|---|
| 34 | Luganil Bordeaux B | Appearance |
| | Additional Wash (after drying) | Fairly even dye coverage with some |
| | Compression with ‘doily emboss’ at | areas on the pigment |
| | 100 kg/cm² | showing on top |
| | Additional pigment coat applied | Good dye penetration |
| | Sample produced in Trial 37 | Feel |
| | FIG. 83A and 83B | Some firm areas but still flexible |
| | | Embossed side is rough whilst the |
| | | underside is soft with some areas of |
| | | roughness |
| | | The finish on this material with the |
| | | emboss and additional pigment |
| | | coat has highlighted some of the |
| | | inconsistencies seen in the material. |
| | | The emboss pattern has remained visible |
| | | on the material once the |
| | | sample was completely dry. However, |
| | | the embossing technique |
| | | requires further exploration. |
| 35 | Luganil Red EB | Appearance |
| | Additional Wash (after drying) | Patchiness across whole sample |
| | Compression with ‘doily emboss’ at | Emboss less visible |
| | 100 kg/cm² | Good dye penetration |
| | Additional pigment coat and an “oily | Feel |
| | pull up’ top coat applied which was | Flexible with firm areas |
| | then kiss plated | Also, some areas on weakness and |
| | Sample produced in Trial 37 | sample is becoming creased in these |
| | FIG. 84A and 84B | areas |
| | | The finishing technique has resulted |
| | | in a loss of depth with the |
| | | embossed pattern. The added top coat |
| | | also appears to be sitting |
| | | irregularly on the surface of the |
| | | sample giving an undesirable |
| | | appearance. |
| 35 | Luganil Yellow G | Appearance |
| | Additional Wash (after drying) | Even dye coverage with some inner |
| | Compression with ‘doily emboss’ at | dye showing through on one |
| | 100 kg/cm² | side. |
| | Additional pigment coat applied | Underside shows dark patches |
| | Sample produced in Trial 39 | Good dye penetration |
| | FIG. 85A and 85B | |
| | | Feel |
| | | Soft on both sides with the embossed |
| | | side giving a soft textured feel. |
| 35 | Luganil Olive Brown N | Appearance |
| | Additional Wash (after drying) | Fairly even dye coverage with some |
| | Compression with ‘doily emboss’ at | dark patches on the underside |
| | 100 kg/cm² | Good dye penetration with a slight |
| | Additional pigment coat applied | lighter appearance in the |
| | Sample produced in Trial 41 | middle |
| | FIG. 86A and 86B | Some creasing observed |
| | | Feel |
| | | Rough texture on the embossed side |
| | | Soft on the underside |
| | | Flexible with some areas of weakness |
| | | Improved emboss retention once dry |
| | | after embossing and drying |

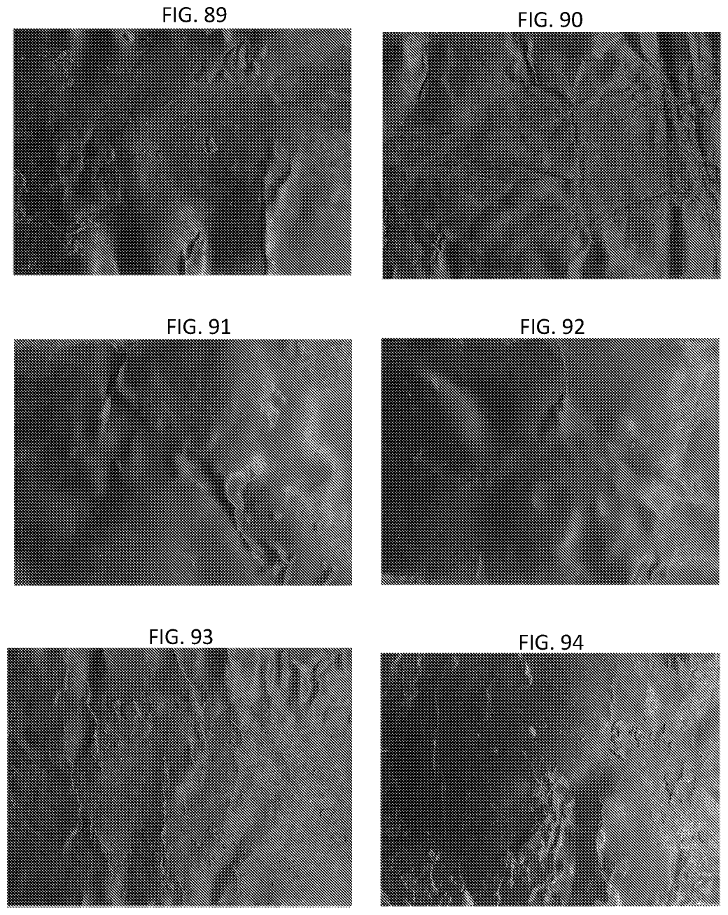

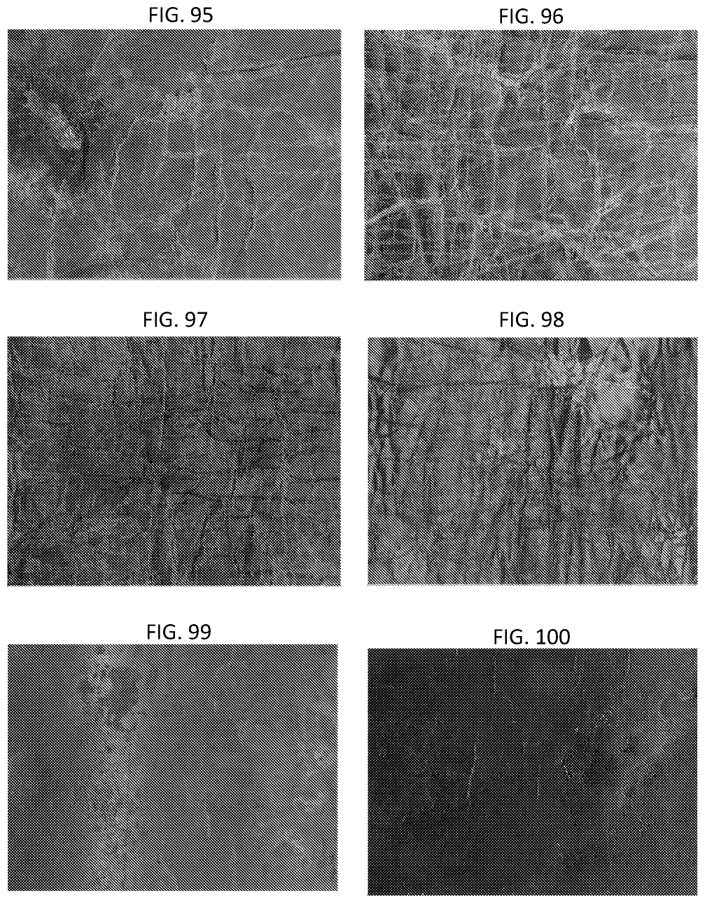

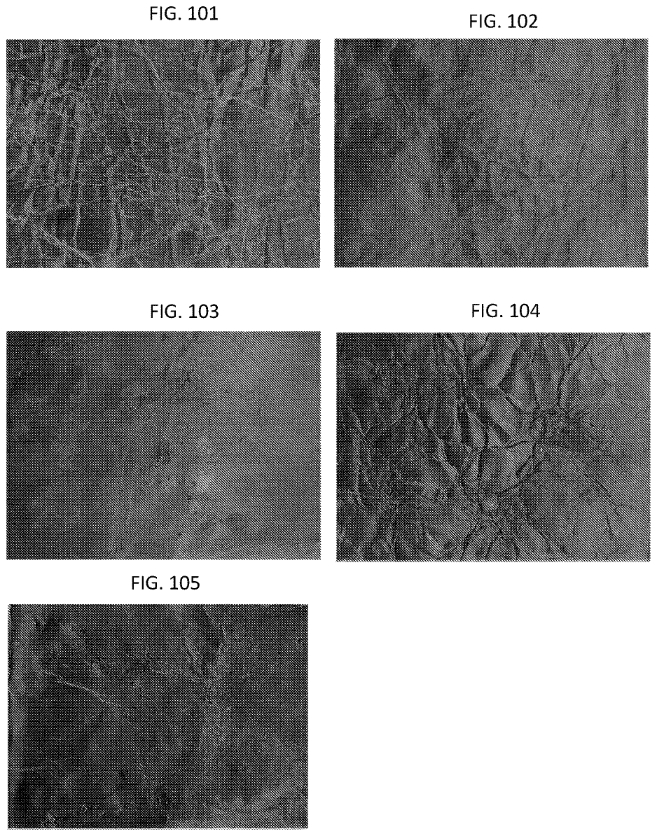

Various protein and wax finishes were also applied to the dyed cultivated mycelium material. FIG. 87 shows an exemplary mycelium material after a pea protein finish. FIG. 88 shows an exemplary mycelium material after an unstirred soya protein finish. FIG. 89 shows an exemplary mycelium material after a stirred soya protein finish. FIG. 90 shows an exemplary mycelium material after a hemp protein finish. FIG. 91 shows an exemplary mycelium material after a 50:50 pea protein to FI 50 finish. FIG. 92 shows an exemplary mycelium material after a 50:50 soya protein to FI 50 finish. FIG. 93 shows an exemplary mycelium material after a pea protein and crosslinker finish. FIG. 94 shows an exemplary mycelium material after Luganil Brown dye and a carnauba flake wax finish. FIG. 95 shows an exemplary mycelium material after Luganil Bordeaux dye, wash, and a carnauba flake wax finish. FIG. 96 shows an exemplary mycelium material after Luganil Yellow dye, wash, and a carnauba liquid wax finish. FIG. 97 shows an exemplary mycelium material after Luganil Brown dye, wash, and a carnauba liquid wax finish. FIG. 98 shows an exemplary mycelium material after a waxy filler, water based PU, and carnauba flake wax finish. FIG. 99 shows an exemplary mycelium material after a 1× coat of pea protein and crosslinker finish. FIG. 100 shows an exemplary mycelium material after a 2× coat of pea protein and crosslinker finish. FIG. 101 shows an exemplary mycelium material after a pea protein, crosslinker, and filler finish without embossing. FIG. 102 shows an exemplary mycelium material after a pea protein, crosslinker, and filler finish with embossing. FIG. 103 shows an exemplary mycelium material after Luganil Red dye, wash, and a pea protein and crosslinker finish. FIG. 104 shows an exemplary mycelium material after Luganil Brown dye, and a glycerine soak, pea protein and crosslinker finish. FIG. 105 shows an exemplary mycelium material after Luganil Bordeaux dye, and a pea protein and crosslinker finish.

1. A method, comprising:
a. generating a cultivated mycelium material;
b. contacting the cultivated mycelium material with a solution comprising one or more proteins to produce a composition comprising the cultivated mycelium material and one or more proteins, wherein the one or more proteins are from a species other than a fungal species from which the cultivated mycelium material is generated; and
c. pressing the cultivated mycelium material.

2. The method of claim 1, wherein the contacting comprises submerging the cultivated mycelium material in the solution.

3. The method of claim 1, wherein the contacting comprises contacting the cultivated mycelium material with the solution in a single step.

4. The method of claim 1, wherein the contacting comprises contacting the cultivated mycelium material with the solution in one or more steps.

5. The method of claim 1, wherein the one or more proteins are from a plant source.

6. The method of claim 5, wherein the plant source is a pea plant.

7. The method of claim 5, wherein the plant source is a soybean plant.

8. The method of claim 1, the solution comprises a dye.

9. The method of claim 8, wherein the composition is colored with the dye to produce a color, and the color of the cultivated mycelium material is substantially uniform on one or more surfaces of the cultivated mycelium material.

10. The method of claim 8, wherein the dye is penetrated throughout the interior of the composition.

11. The method of claim 8, wherein the dye is selected from the group consisting of: an acid dye, a direct dye, a synthetic dye, a natural dye, and a reactive dye.

12. The method of claim 1, wherein the solution comprises a plasticizer.

13. The method of claim 12, wherein the plasticizer is selected from the group consisting of oil, glycerin, and fat liquor.

14. The method of claim 12, wherein the composition is flexible.

15. The method of claim 1, wherein the one or more proteins are crosslinked.

16. The method of claim 1, wherein the one or more proteins are crosslinked with transglutaminase.

17. The method of claim 1, wherein the solution comprises an enzyme.

18. The method of claim 17, wherein the enzyme comprises transglutaminase.

19. The method of claim 1, wherein the pressing comprises pressing the cultivated mycelium material to a thickness of 0.1 inch to 0.5 inch.

20. The method of claim 19, wherein the pressing comprises pressing the cultivated mycelium material to a thickness of 0.25 inch.

21. The method of claim 1, wherein the pressing is repeated one or more times.

22. The method of claim 1, wherein the pressing comprises pressing the cultivated mycelium material to a thickness of 0.25 inch.

23. The method of claim 1, wherein the pressing comprises pressing the cultivated mycelium material with a roller.

24. The method of claim 1, wherein the solution comprises tannins.

25. The method of claim 1, further comprising incubating the composition.

26. The method of claim 25, wherein the incubating comprises incubating the composition at a set temperature for a set amount of time.

27. The method of claim 26, wherein the set temperature is 40° C.

28. The method of claim 1, further comprising drying the composition.

29. The method of claim 1, further comprising applying a finishing agent to one or more surfaces of the composition.

30. The method of claim 29, wherein the finishing agent is selected from the group consisting of: urethane, wax, nitrocellulose, or a plasticizer.

31. A composition comprising a cultivated mycelium material and one or more proteins, wherein the one or more proteins are from a species other than a fungal species from which the cultivated mycelium material is generated.

32. The composition of claim 30, wherein the one or more proteins are from a plant source.

33. The composition of claim 32, wherein the plant source is a pea plant.

34. The composition of claim 32, wherein the plant source is a soybean plant.

35. The composition of claim 30, wherein the composition comprises a dye.

36. The composition of claim 35, wherein the dye is selected from the group consisting of: an acid dye, a direct dye, a synthetic dye, a natural dye, and a reactive dye.

37. The composition of claim 30, wherein the composition comprises a plasticizer.

38. The composition of claim 37, wherein the plasticizer is selected from the group consisting of oil, glycerin and fat liquor.

39. The composition of claim 37, wherein the composition is flexible.

40. The composition of claim 30, wherein the one or more proteins are crosslinked.

41. The composition of claim 30, wherein the one or more proteins are crosslinked with transglutaminase.

42. The composition of claim 30, wherein the composition comprises an enzyme.

43. The composition of claim 42, wherein the enzyme comprises transglutaminase.

44. A composition comprising a cultivated mycelium material colored with a dye to produce a color, and wherein the color of the cultivated mycelium material is substantially uniform on one or more surfaces of the cultivated mycelium material.

45. The composition of claim 44, wherein the dye is selected from the group consisting of: an acid dye, a direct dye, a synthetic dye, a natural dye, and a reactive dye.

46. The composition of claim 44, wherein the composition comprises one or more proteins that are from a species other than a fungal species from which the cultivated mycelium material is generated.

47. The composition of claim 46, wherein the one or more proteins are from a plant source.

48. The composition of claim 47, wherein the plant source is a pea plant.

49. The composition of claim 47, wherein the plant source is a soybean plant.

50. The composition of claim 44, wherein the dye is penetrated throughout the interior of the composition.

51. The composition of claim 44, wherein the composition comprises a plasticizer.

52. The composition of claim 51, wherein the plasticizer is selected from the group consisting of oil, glycerin, and fat liquor.

53. The composition of claim 51, wherein the composition is flexible.

54. The composition of claim 44, wherein the composition comprises tannins.

55. The composition of claim 44, wherein the composition comprises a finishing agent applied to one or more surfaces of the composition.

56. The composition of claim 55, wherein the finishing agent is selected from the group consisting of: urethane, wax, nitrocellulose, or a plasticizer.

57. An article of footwear, comprising:
a. an upper;
b. a lasting board affixed with the upper to define an interior foot-receiving cavity therewith;
c. an outsole coupled with the upper opposite the lasting board;
d. wherein the upper includes at least a portion of a mycelium material that includes one or more proteins derived from an organism other than mycelium.

58. The article of footwear of claim 57, wherein the upper comprises a plurality of portions of the mycelium material in respective implementations thereof having different physical properties.

59. The article of footwear of claim 58, wherein the different physical properties are selected to correlate with desired characteristics of the corresponding locations of the portions within the upper.

60. The article of footwear of claim 59, wherein one of the portions of the mycelium material includes a vamp, the respective implementation of the mycelium material having higher relative flexibility compared to at least one of the portion.

61. The article of footwear of claim 59, wherein one of the portions of the mycelium material includes a heel counter, the respective implementation of the mycelium material having higher relative rigidity compared to at least one of the portion.

62. The article of footwear of claim 57, wherein the mycelium material is at least one of tanned and dyed to resemble leather.

63. The article of footwear of claim 57, further including a midsole affixed with the lasting board, the outsole being affixed with the midsole so as to be coupled with the upper.

64. The article of footwear of claim 57, wherein the upper comprises a plurality of discrete portions of the mycelium material.

65. The article of footwear of claim 64, wherein the portions are assembled together using at least one of: topstitching, felled stitching, and stitch and turn construction.

66. The article of footwear of claim 64, wherein the portions are assembled together using at least one of: solvent-based adhesive, UV curing adhesive, heat-activated adhesive, and water-based adhesive.

67. The article of footwear of claim 64, wherein at least one of the portions is split to resemble suede leather.

68. The article of footwear of claim 64, wherein at least one of the portions includes an edge thinned by skivving.

69. The article of footwear of claim 64, wherein the portions are assembled together using heat bonding.

70. The article of footwear of claim 64, wherein the upper further includes at least one additional portion of a textile material.

71. The article of footwear of claim 64, wherein the textile material is thermoplastic and is affixed with at least one of the portions of the mycelium material by heat bonding.

72. The article of footwear of claim 57, wherein the upper includes interfacing assembled with a portion thereof.

73. The article of footwear of claim 57, including perforations along a portion thereof.

74. The article of footwear of claim 73, wherein the perforations vary in at least one of size and relative spacing over an area of the upper.

75. The article of footwear of claim 57, wherein the upper is laser etched along a portion thereof.

76. The article of footwear of claim 57, wherein the upper includes at least one reinforcing portion injection molded thereon.

77. The article of footwear of claim 57, wherein the upper includes at least one 3-D printed element fused therewith.

78. The article of footwear of claim 57, wherein at least a portion of the upper includes at least one portion molded in a three dimensional shape.

79. The article of footwear of claim 57, wherein the upper is comprised of a single molded piece of the mycelium material.

80. The article of footwear of claim 57, wherein the mycelium material includes a plurality of bonded layers of the mycelium material in respective implementations thereof having different physical properties.

81. The article of footwear of claim 57, wherein at least one of the lasting board and the out-sole includes at least a portion of the mycelium material.

82. An athletic sneaker, comprising:
a. an upper including at least a portion of a mycelium material that includes one or more proteins derived from an organism other than mycelium;
b. a lasting board affixed with the upper to define an interior foot-receiving cavity therewith;
c. a midsole of a foam material and affixed with the lasting board; and
d. an outsole of a rubber material and affixed with the midsole opposite the lasting board;
e. wherein the mycelium material is at least one of tanned and dyed to resemble leather, and the upper is configured and assembled to resemble athletic footwear of leather.

83. An athletic sneaker, comprising:
a. an upper including at least a portion of a mycelium material that includes one or more proteins derived from an organism other than mycelium;
b. a lasting board affixed with the upper to define an interior foot-receiving cavity therewith;
c. a midsole of a foam material and affixed with the lasting board; and
d. an outsole of a rubber material and affixed with the midsole opposite the lasting board;
e. wherein the upper includes at least one portion molded in a three dimensional shape.
